SEQUENCE LISTING (48 sequences)

SEQ ID NO: 1 - protein, 527 aa, Neisseria gonorrhoeae
  MsrAB from Neisseria gonorrhoeae, with His tag/WQ protease site
HHHHHHSSGLVPRGSHMWELQLALGACSPKIVDAGAATVPHTLSTLKTADNRPASVYLKKDKPTLIKFWASWCPLCLSELGQAEKWAQDAKFSSANLITVASPGFLHEKKDGEFQKWYAGLNYPKLPVVTDNGGTIAQNLNISVYPSWALIGKDGDVQRIVKGSINEAQALALIRNPNADLGSLKHSFYKPDTQKKDSAIMNTRTIYLAGGCFWGLEAYFQRIDGVVDAVSGYANGNTENPSYEDVSYRHTGHAETVKVTYDADKLSLDDILQYYFRVVDPTSLNKQGNDTGTQYRSGVYYTDPAEKAVIAAALKREQQKYQLPLVVENEPLKNFYDAEEYHQDYLIKNPNGYCHIDIRKADEPLPGKTKAAPQGKGFDAATYKKPSDAELKRTLTEEQYQVTQNSATEYAFSHEYDHLFKPGIYVDVVSGEPLFSSADKYDSGCGWPSFTRPIDAKSVTEHDDFSFNMRRTEVRSRAADSHLGHVFPDGPRDKGGLRYCINGASLKFIPLEQMDAAGYGALKGKVK

SEQ ID NO: 2 - protein, 527 aa, Neisseria meningitides
  MsrAB from Neisseria meningitides, with His tag/WQ protease site
HHHHHHSSGLVPRGSHMWELQLALGACSPKIVDAGAATVPHTLSTLKTADNRPASVYLKKDKPTLIKFWASWCPLCLSELGQTEKWAQDAKFSSANLITVASPGFLHEKKDGDFQKWYAGLNYPKLPVVTDNGGTIAQSLNISVYPSWALIGKDSDVQRIVKGSINEAQALALIRDPNADLGSLKHSFYKPDTQKKDSKIMNTRTIYLAGGCFWGLEAYFQRIDGVVDAVSGYANGNTKNPSYEDVSYRHTGHAETVKVTYDADKLSLDDILQYFFRVVDPTSLNKQGNDTGTQYRSGVYYTDPAEKAVIAAALKREQQKYQLPLVVENEPLKNFYDAEEYHQDYLIKNPNGYCHIDIRKADEPLPGKTKTAPQGKGFDAATYKKPSDAELKRTLTEEQYQVTQNSATEYAFSHEYDHLFKPGIYVDVVSGEPLFSSADKYDSGCGWPSFTRPIDAKSVTEHDDFSYNMRRTEVRSHAADSHLGHVFPDGPRDKGGLRYCINGASLKFIPLEQMDAAGYGALKGKVK

SEQ ID NO: 3 - protein, 148 aa, Artificial Sequence
  Description of Artificial Sequence: Synthetic polypeptide
  TurboLuc
MEAEAERGKLPGKKLPLEVLIELEANARKAGCTRGCLICLSKIKCTAKMKKYIPGRCADYGGDKKTGQAGIVGAIVDIPEISGFKEMEPMEQFIAQVDRCADCTTGCLKGLANVKCSDLLKKWLPGRCATFADKIQSEVDNIKGLAGD

SEQ ID NO: 4 - protein, 23 aa, Artificial Sequence
  Description of Artificial Sequence: Synthetic Peptide 1
  MOD_RES (9)..(9): Carbamidomethyl C
  MOD_RES (21)..(21): Oxidized M
RTTSFAESCKPVQQPSAFGSMKV

SEQ ID NO: 5 - protein, 23 aa, Artificial Sequence
  Description of Artificial Sequence: Synthetic Peptide 1, reduced
  MOD_RES (9)..(9): Carbamidomethyl C
RTTSFAESCKPVQQPSAFGSMKV

SEQ ID NO: 6 - protein, 23 aa, Artificial Sequence
  Description of Artificial Sequence: Synthetic Peptide 2
  MOD_RES (4)..(4): Phosphorylated S
  MOD_RES (9)..(9): Carbamidomethyl C
  MOD_RES (21)..(21): Oxidized M
RTTSFAESCKPVQQPSAFGSMKV

SEQ ID NO: 7 - protein, 23 aa, Artificial Sequence
  Description of Artificial Sequence: Synthetic Peptide 2, reduced
  MOD_RES (4)..(4): Phosphorylated S
  MOD_RES (9)..(9): Oxidized C
RTTSFAESCKPVQQPSAFGSMKV

SEQ ID NO: 8 - protein, 23 aa, Artificial Sequence
  Description of Artificial Sequence: Synthetic Peptide 3
  MOD_RES (9)..(9): Phosphorylated S
  MOD_RES (13)..(13): Oxidized M
RTESITATSPASMVGGKPGSFRV

SEQ ID NO: 9 - protein, 23 aa, Artificial Sequence
  Description of Artificial Sequence: Synthetic Peptide 3, reduced
  MOD_RES (9)..(9): Phosphorylated S
RTESITATSPASMVGGKPGSFRV

SEQ ID NO: 10 - protein, 506 aa, Neisseria gonorrhoeae
  MsrAB from Neisseria gonorrhoeae
LALGACSPKIVDAGAATVPHTLSTLKTADNRPASVYLKKDKPTLIKFWASWCPLCLSELGQAEKWAQDAKFSSANLITVASPGFLHEKKDGEFQKWYAGLNYPKLPVVTDNGGTIAQNLNISVYPSWALIGKDGDVQRIVKGSINEAQALALIRNPNADLGSLKHSFYKPDTQKKDSAIMNTRTIYLAGGCFWGLEAYFQRIDGVVDAVSGYANGNTENPSYEDVSYRHTGHAETVKVTYDADKLSLDDILQYYFRVVDPTSLNKQGNDTGTQYRSGVYYTDPAEKAVIAAALKREQQKYQLPLVVENEPLKNFYDAEEYHQDYLIKNPNGYCHIDIRKADEPLPGKTKAAPQGKGFDAATYKKPSDAELKRTLTEEQYQVTQNSATEYAFSHEYDHLFKPGIYVDVVSGEPLFSSADKYDSGCGWPSFTRPIDAKSVTEHDDFSFNMRRTEVRSRAADSHLGHVFPDGPRDKGGLRYCINGASLKFIPLEQMDAAGYGALKGKVK

SEQ ID NO: 11 - protein, 506 aa, Neisseria meningitides
  MsrAB from Neisseria meningitides
LALGACSPKIVDAGAATVPHTLSTLKTADNRPASVYLKKDKPTLIKFWASWCPLCLSELGQTEKWAQDAKFSSANLITVASPGFLHEKKDGDFQKWYAGLNYPKLPVVTDNGGTIAQSLNISVYPSWALIGKDSDVQRIVKGSINEAQALALIRDPNADLGSLKHSFYKPDTQKKDSKIMNTRTIYLAGGCFWGLEAYFQRIDGVVDAVSGYANGNTKNPSYEDVSYRHTGHAETVKVTYDADKLSLDDILQYFFRVVDPTSLNKQGNDTGTQYRSGVYYTDPAEKAVIAAALKREQQKYQLPLVVENEPLKNFYDAEEYHQDYLIKNPNGYCHIDIRKADEPLPGKTKTAPQGKGFDAATYKKPSDAELKRTLTEEQYQVTQNSATEYAFSHEYDHLFKPGIYVDVVSGEPLFSSADKYDSGCGWPSFTRPIDAKSVTEHDDFSYNMRRTEVRSHAADSHLGHVFPDGPRDKGGLRYCINGASLKFIPLEQMDAAGYGALKGKVK

SEQ ID NO: 12 - protein, 506 aa, Neisseria lactamica
  Methionine sulfoxide reductase, Neisseria lactamica
FALGACSPKIADAEAATVPHTLSTLKTADNRPADVYLKKDKPTLIKFWASWCPLCLSELGQTEKWAQDAKFGSANLITVASPGFLHEKKDGDFQKWYAGLNYPKLPVVTDNGGTIAQSLNISVYPSWALIGKDGDVQRIVKGSINEAQALALIRDPNADLGSLKHSFYKPDTQKKDSKIMNTRTIYLAGGCFWGLEAYFQRIDGVVDAVSGYANGNTKNPSYEDVSYRHTGHAETVKVTYDADKLSLDDILQYYFRVVDPTSLNKQGNDTGTQYRSGVYYTDPAEKAVIAAALKREQQKYKLPLVVENEPLKNFYDAEEYHQDYLIKNPNGYCHIDIRKADEPLPGKTKTAPQGKGFDAATYKKPSDAELKRTLTEEQYQVTQKSATEYAFSHEYDHLFKPGIYVDVVSGEPLFSSADKYDSGCGWPSFTRPIDAKSVTEHDDFSFNMRRTEVRSHAADSHLGHVFPDGPRDKGGLRYCINGASLKFIPLEQMDAAGYGALKGKVK

SEQ ID NO: 13 - protein, 506 aa, Neisseria polysaccharea
  Methionine sulfoxide reductase, Neisseria polysaccharea
FALGACSPKIADAEAATVPHTLSTLKTADNRPADVYLKKDKPTLIKFWASWCPLCLSELGQTEKWAQDAKFGSANLITVASPGFLHEKKDGDFQKWYAGLNYPKLPVVTDNGGTIAQSLNISVYPSWALIGKDGDVQRIVKGSINEAQALALIRDPNADLGSLKHSFYKPDTQKKDSKIMNTRTIYLAGGCFWGLEAYFQRIDGVVDAVSGYANGNTKNPSYEDVSYRHTGHAETVKVTYDADKLSLDDILQYYFRVVDPTSLNKQGNDTGTQYRSGVYYTDPAEKTVIAAALKREQQKYKLPLVVENEPLKNFYDAEEYHQDYLIKNPNGYCHIDIRKADEPLPGKTKTAPQGKGFDAATYKKPSDAELKRTLTEEQYQVTQHSATEYAFSHEYDHLFKPGIYVDVVSGEPLFSSADKYDSGCGWPSFTRPIDAKSVTEHDDFSYNMRRTEVRSHAADSHLGHVFPDGPRDKGGLRYCINGASLKFIPLEQMDAAGYGALKGKVK

SEQ ID NO: 14 - protein, 506 aa, Neisseria flavescens
  Methionine sulfoxide reductase, Neisseria flavescens
FALGACSPKTADAGAATVPHTLSTLKTADNRPAGVYLKKDKPTLIKFWASWCPLCLSELGQTEKWAQDAKFGSANLITVASPGFLHEKKDGDFQKWYAGLNYPKLPVVTDNGGTIAQSLNISVYPSWALIGKDGDVQRIVKGSINEAQALALIRDPNADLGSLKHSFYKPDTQKKDSKIMNTRTIYLAGGCFWGLEAYFQRIDGVVDAVSGYANGNTKNPSYEDVSYRHTGHAETVKVTYDADRLSLDDILQYYFRVVDPTSLNKQGNDTGTQYRSGVYYTDPAEKAVIAAALKREQQKYKLPLVVENEPLKNFYDAEEYHQDYLIKNPNGYCHIDIRKADEPLAGKTQTAPQGKGFDAATYKKPSDAELKRTLTEEQYQVTQHSATEYAFSHEYDHLFKPGIYVDVVSGEPLFSSADKYDSGCGWPSFTRPIDAKSVTEHNDFSYNMRRTEVRSHAADSHLGHVFPDGPRDKGGLRYCINGASLKFIPLEQMDAAGYGALKGKVK

SEQ ID NO: 15 - protein, 506 aa, Neisseria sicca
  Methionine sulfoxide reductase, Neisseria sicca
FALGACSPKIADAEAATVPHTLSTLKTADNRPASVYLKKDKPTLIKFWASWCPLCLSELGQTEKWAQDKRFSSANLITVASPGFLHEKKDGDFQKWYAGLNYPKLPVVTDNGGTIAQSLNISVYPSWALIGKDGDVQRIVKGSINEAQALALIRDPNADLGRLKNAFYKPDTQKKDSKIMNTRTIYLAGGCFWGLEAYFQRIDGVVDAVSGYANGKTKNPSYEDVSYRDTGHAETVKVTYDADKLSLDDILQYYFRVVDPTSLNKQGNDTGTQYRSGVYYTDPAEKAVIAAALKREQQKYKQPLVVENEPLKNFYDAEEYHQDYLIKNPNGYCHIDIRKADEPLPGKTKAAPQGKGFDAATYKKPSDAELKRILTEEQYQVTQKSATEYAFSHEYDHLFKPGIYVDVVSGEPLFSSADKFDSGCGWPSFTRPINAAAVTEHDDFSYNMRRTEVRSHAADSHLGHVFPDGPKDKGGLRYCINGASLKFIPLEQMDAAGYGALKGKVK

SEQ ID NO: 16 - protein, 506 aa, Neisseria macacae
  Methionine sulfoxide reductase, Neisseria macacae
FALGACSPKIADAEAATVPHTLSTLKTADNRPASVYLRKDKPTLIKFWASWCPLCLSELGQTEKWAQDKRFSSANLITVASPGFLHEKKDGDFQKWYAGLNYPKLPVVTDNGGTIAQSLNISVYPSWALIGKDGDVQRIVKGSINEAQALALIRDPNADLGRLKNAFYKPDTQKKDSKIMNTRTIYLAGGCFWGLEAYFQRIDGVVDAVSGYANGKTKNPSYEDVSYRDTGHAETVKVTYDADKLSLDDILQYYFRVVDPTSLNKQGNDTGTQYRSGVYYTDPAEKAVIAAALKREQQKYKQPLVVENEPLKNFYDAEEYHQDYLIKNPNGYCHIDIRKADEPLPGKTKAAPQGKGFDAATYKKPSDAELKRILTEEQYQVTQKSATEYAFSHEYDHLFKPGIYVDVVSGEPLFSSADKFDSGCGWPSFTRPINAAAVTEHDDFSYNMRRTEVRSHAADSHLGHVFPDGPKDKGGLRYCINGASLKFIPLEQMDAAGYGALKGKVK

SEQ ID NO: 17 - protein, 506 aa, Neisseria mucosa
  Methionine sulfoxide reductase, Neisseria mucosa
FALGACSPKIADAEAATVPHTLSTLKTADNRPASVYLKKDKPTLIKFWASWCPLCLSELGQTEKWAQDKRFSSANLITVASPGFLHEKKDGDFQKWYAGLNYPKLPVVTDNGGTIAQSLNISVYPSWALIGKDGDVQRIVKGSINEAQALALIRDPNADLGRLKNAFYKPDTQKKDSKIMNTRTIYLAGGCFWGLEAYFQRIDGVVDAVSGYANGKTKNPSYEDVSYRDTGHAETVKVTYDADKLSLDDILQYYFRVVDPTSLNKQGNDTGTQYRSGVYYTDPAEKAVIAAALKREQQKYKQPLVIENEPLKNFYDAEEYHQDYLIKNPNGYCHIDIRKADEPLPGKTKAAPQGKGFDAATYKKPSDAELKRILTEEQYQVTQKSATEYAFSHEYDHLFKPGIYVDVVSGEPLFSSADKFDSGCGWPSFTRPINAAAVTEHDDFTYNMRRTEVRSHAADSHLGHVFADGPQDKGGLRYCINGASLKFIPLEQMDAAGYGALKGKVK

SEQ ID NO: 18 - protein, 506 aa, Neisseria flavescens
  Methionine sulfoxide reductase, Neisseria flavescens
LALGACSSKIMDTEAATVPQALSSLKTPDNRPASVFLKKDKPTLIKFWASWCPLCLSELGQTEKWAQDTKFGSANLITVASPGFLHEKKDGDFQKWYAGLNYPKLPVVTDNGGTIAQSLNISVYPSWALIGKDGDVQRIVKGSINEAQALALIRDPNADLGRLKNSFYKPDTQKKDSAIMNTRTIYLAGGCFWGLEAYFQRIDGVVDAVSGYANGKTENPSYEDVSYRDTGHAETVKVTYDADKLSLDDILQYFFRVVDPTSLNKQGNDTGTQYRSGVYYTDPAEKAVIAAALKREQQKYKQPLVVENEPLKNFYDAEEYHQDYLIKNPNGYCHIDIRKADEPLPGKTKAAPQGKGFDAATYKKPGAAELKRLLTEEQYQVTQNSATEYAFSHEYDHLFKPGIYVDVVSGEPLFSSADKFDSGCGWPSFTHPINASAVTEHDDFSYNMRRTEVRSHAADSHLGHVFPDGPKDKGGLRYCINGASLKFIPLEQMDAAGYGALKGKVK

SEQ ID NO: 19 - protein, 565 aa, Lautropia mirabilis
  Methionine sulfoxide reductase, Lautropia mirabilis
MIMRRLLTPRNLLLLVLLAVMFWSFYSGASPSHGTPPASADKAATAQGGGAAGAAQASDGAPEQPVGLPLAYLQKLKDVADKPATTYIKPGRPTLVKFWASWCPLCLSELADTNAWATDERFSSAVNLVTLASPGFLHEKPQADFVTWYGGLDYPAMPVLLDVGGLLARQLGVRVYPSWVLLDADGGVARVVRGRLSEAQALALIEDPEADLARLAQAERASFYQPDSQKSSKVMNTKTIYLAGGCFWGVEAYFQRIPGVVDAVSGYANGRTQNPSYEDVIRGAGHAETVKVTYDADRLSLADILQYYFRIIDPTSLNKQGNDRGAQYRTGVYYTDAADKATIQQALDALQQKYSRPLVVENLPLQNFYEAEEYHQDYLAKNPNGYCHIDVRKADEPLPGKPAGNPPAAAAVGRGFDVASYRKASDAELKQRLSAEQYRVTQQSGTERAFTHEYDHLFAPGIYVDVVSGQPLFSSKDKFDSGCGWPSFTRPIQPSAVTEHEDLSYNMRRVEVRSQAADSHLGHVFPDGPRDKGGLRYCINGASLRFIPLEKMAEEGYGNLVDAVK

SEQ ID NO: 20 - protein, 542 aa, Cardiobacterium hominis
  Methionine sulfoxide reductase, Cardiobacterium hominis
MKNPRQTLCSLIACVLFAGAVAPLPVLADAHASRAEAPLPHQLQQRLLALKDPRDQPAADYLDQSKPTLIKFWASWCPLCLATLEETQAWRGDKAFAGVNLVTIASPDHLGENDEATFKEWYRGLDYPNLPVLVNNGGDIARDIGVAVYPSWALLDKNGNVARVIKGHINREQALALLANPQAELAQPAQKFYKPKPKGATNMNTKTIHLAGGCFWGLEAYFERIPGVVDAVSGYANGKTKNPSYEDVSHRGTGHAETVKVTYDPERISLDDLLRYYFRVVDPTSLNQQGNDRGVQYRSGVYYTDPAERATIEKAFAEEQKKHQKPLVVENLPLDNFYEAEEYHQDYLAKNPNGYCHIDIRKADIPLEKPAATAPAPAQTDANGEPVIDAAKYHKPDAAELKQKLDAQAYEVTQNSATERAFSHEYDHLFAPGLYVDVVSGEPLFSSADKFQSGCGWPSFTKPINRAVVTEHDDTSYNMHRTEIRSRVADAHLGHVFPDGPKDKGGLRYCINGASLKFIPLAEMEKAGYGDLVDAVKKGEKL

SEQ ID NO: 21 - protein, 542 aa, Unknown
  Description of Unknown: Gammaproteobacteria sequence
  Methionine sulfoxide reductase, Gammaproteobacteria
MKNPRQTLCSLIACVLFAGAVAPLPVLADAHASRAEAPLPHQLQQRLLALKDPRDKPAADYLDQSKPTLIKFWASWCPLCLATLEETQAWRGDKAFAGVNLVTIASPDHLGENDEATFKEWYRGLDYPNLPVLVNNGGDIARDIGVAVYPSWALLDKNGNVARVIKGHINREQALALLANPQAELAQPAQKFYKPKPKGATNMNTKTIHLAGGCFWGLEAYFERIPGVVDAVSGYANGKTKNPSYEDVSHRGTGHAETVKVTYDPERISLDDILRYYFRVVDPTSLNQQGNDRGVQYRSGVYYTDPAERATIEKAFAEEQKKHQKPLVVENLPLDNFYEAEEYHQDYLAKNPNGYCHIDIRKADIPLEKPAATAPAPAQTDANGEPVIDATKYHKPDAAELKKKLDAQAYEVTQNSATERAFSHEYDHLFAPGLYVDVVSGEPLFSSADKFQSGCGWPSFTKPINRAVVTEHDDTSYNMHRTEIRSRVADAHLGHVFPDGPKDKDGLRYCINGASLKFIPLAEMAQAGYGDLVDAVKKGEKL

SEQ ID NO: 22 - protein, 504 aa, Marinospirillum insulare
  Methionine sulfoxide reductase, Marinospirillum insulare
MKSPLAKANKPNFFQQLTQLQPVTNGSSNMQFNNNRPTLVKLWASWCPLCLSELELTQSWANDPDFAQVNLTTLASPGVLGELSLEEFKQWYAGLDYPDLPLQLDPSGELVKKLGVQVYPSWAVLDAQGNLQRVVKGSINKAQALALIANPEADLKQLQTTFYQPKQPAQALPINTQSVYLAGGCFWGVEGYFERIDGVVDAVSGYANGRTENPSYEDVIYRNTGHAETVKVTYNSDKLSLDDILVYFFRIIDPTSLNKQGNDRGTQYRTGIYTTDPAEQRLVATALARLEEEYTQPILVENLPLSGFYEAEEYHQDYLLKNPNGYCHVDLNKADIPLPNQLTNQSTDKNTPKPFDPNNFQKPDTASLKQRLTSEQFHVTQNNGTERAFTHEYDDLFEPGLYVDIVSGEPLFSSKDKYQAGCGWPSFVKPIEENALVEVVDTSYNMRRIEVRSRLADSHLGHVFPDGPKDRGGLRYCINGASLKFIPLAEMQAQGYGDWQALIN

SEQ ID NO: 23 - protein, 510 aa, Pelistega indica
  Methionine sulfoxide reductase, Pelistega indica
MPFLYFLRTIILGIMALYSSTLFAQTINFNALKDINNQKANFYIKNNKPTVVKFWASWCPLCLGELEQTEQWVQDKDFAMVNMVTLASPGYLGEKKAADFSQWALSLPYKKLPILIDTEQTIAKSLNIRVYPSWVLLDSNGQLVKVVKGTLSKEQLLGVIKNPDAPIQKASTTFYKADTNSEHKKPIRTETIYLAGGCFWGLEGYFQRIPGVIDAVSGYANGNTQNPSYEDVVYRHTGHAETVKVTYDIDKLSFADILEYYFRVIDPTSLNQQGNDKGTQYRTGIYYTKADYQPLIAEAIKKEQTKYKKPIVVENKPLANFYPAEEYHQDYLLKNPNGYCHIDLNKADEPLSTPSPKGFNMKEYKKPSQSELRQRLTPEQYRVTQESGTEYAFSHEYDELFAPGLYVDIVSGQPLFSSDDKFNSHCGWPSFTQPIEKTVVTEHKDFSHNMYRIEVRSQAADSHLGHVFPDGPADRGGLRYCINGASLRFIPYADLDKEGYGEWKDKIKQK

SEQ ID NO: 24 - protein, 526 aa, Basilea psittacipulmonis
  Methionine sulfoxide reductase, Basilea psittacipulmonis
MKKIFALCVTLGIALVTLAFAKLPNSSTDKATQGADDKAFTYLLSLDDIHQQPAKQLIDTNRPTLVKLWASWCSSCLSELDEVEAWSKDKRFKAINFVTVVSPSLYSEKNKDDFTKWFLSLDYPQTKVLLDTKGTLSRTLNIRAYPSWALFDEKGHLVRVIKGSISKVQALALIDNPQADLKSVQEKANRVTKKEVIDPMYQKTIYLAGGCFWGVEAYFERIDGVIDAVSGYANGRTENPKYEDVIYRHTGHAETVKVTFDTRRLSLADILQYYFRVIDPTSLNKQGNDRGTQYRTGVYYTDEKDKAVIDAALANEQKKYTKPLVVENLPLRNFYLAEDYHQDYLKKNPNGYCHIDISLADRPLERGTNIDKPVRFWETYEKPSDNELRQQLSNEQYRITQKNGTEYAFSHAYDHLFEPGLYVDIVSGEPLFTSTDKYDSGCGWPSFTQPIQAQAITEHEDLSYNMRRIEVRSRYADSHLGHVFPDGPSDKGGLRYCINGASLRFIPLEQMAAEGYAEFIPLIKKP

SEQ ID NO: 25 - protein, 544 aa, Oligella ureolytica
  Methionine sulfoxide reductase, Oligella ureolytica
MVQKIPHFFLSILFLTLTVLSLPAQSFSFSTKQHLGPRLEKLNDVQGVKATEFLQTSRPTLVKFWASWCPLCLATLEETRDWRLDPDFSNTDIVTLASPGYLKEQSPKDFRQWYQGVNIEHLPVLVNDGGDLTREIGVSVYPSWALLDAQGRLQRVIKGHITKEQALGLIADKDFDIQRSAPTFYRPSTDTAQQQKDKSNLMNSKEIYLAGGCFWGVEAYFERIPGVLDAISGYANGNTQNPTYEQVIYMGTGHAETVKVVYDPERVDLETILRHFFRIIDPTSLNRQGNDRGTQYRTGIYYTDASDAALITAALAREQSKWQKPLVVENEALDAFYVAEEYHQDYLAKNPNGYCHVDLNLVDQPLEKEEFELEMKSGENTQTMQNLKSEIRINPADYSVPSDEELRQKLSPLEYQVTQQNATERAFTHSYDNLYEPGIYVDIVSGEPLFSSDDKYDSGCGWPSFTKPIVPEVVTEHLDTTYNMQRIETRSRVADAHLGHVFPDGPRDRGGLRYCINGASLKFIPKAEMAAAGYGDLLPLVSDK

SEQ ID NO: 26 - protein, 545 aa, Unknown
  Description of Unknown: Alcaligenaceae sequence
  Methionine sulfoxide reductase, Alcaligenaceae
MHTLFRILSTLLFLSLSFFSFSAHSVGVSSQPHVGQRIAKLKDFQDKPATDYLKKGQPSLVKFWASWCPLCLATLEETRDWRLDPDFAGVNIISLASPGYLNEQSPKEFRQWYRGVNFDNLPVIVNDGGELTRAIGISAYPSWGLIDAEGRLQRVIKGHITKEQALALVADKDYEIKRKTPDFYRPSKDTAQQQKDKANLMKTKEIYLAGGCFWGVEAYFERIPGVVNAVSGYANGKTRQPTYEQVIYMNTGHAETVKVVYDPERIDLETILRHYLRIIDPTSLNRQGNDRGTQYRTGIYYSEPSDKDIITAVLAREQSKWERPIVVENQPLIAFDEAEEYHQDYLAKNPNGYCHIDLNLVDKPLAEEKTPLNLQNGSNTKAMQEHNAQSSITVNPADYHVPKEEELRKTLSPLSYQVTQQNATERAFTHPYDHLFEAGIYVDIVSGEPLFSSDDKYDSGCGWPSFTKPIVPEVITEHLDTSYNMQRIETRSRVADAHLGHVFPDGPRDRGGLRYCINGAALKFIPKAEMEAAGYGYLLPLVSDK

SEQ ID NO: 27 - protein, 595 aa, Psychrobacter piscatorii
  Methionine sulfoxide reductase, Psychrobacter piscatorii
MSYKNNQKNSNHEEIKKPRSSSWLKNVSAFSMTTVLSAGILVACGQMSNAESSASSSTKSGSQNTGVTSSRDMLPSDMLKQMQALPQLTKGLGDTGAAVIDPNKPTLVKFWASWCPLCLGTLEETETWRTDPKFSGLNVVTVASPGHLNEKADGEFSTWYAGVQADYPKLPVLTDPSGELINKLGVQVYPSWAILDKNGNLVHLVKGNISAEQAYALAENAGNGFAELKAGNAKPANAQASDNNKIETIKQKDGVYYNETGKPINTRSIYLAGGCFWGVEAYMERVDGVIDAVSGYANGDTANPSYEQVIRGSGHAETVKVTYDADKTDLDTILKYYFRVVDPTSLNKQGNDRGVQYRSGVYYTDKEDKAVIDAALKRVQSKYEQKVVVENEPLDNFYLAEMYHQDYLAKNPNGYCHIDLSLADDKPEGAARTKLAPVETIAETLDPKRYAKFDKDALKNTLTKAQYNITQEAGTERAFSHEYDDLFAPGIYVDVVSGEPLFLSTDKYQSGCGWPSFTKPIDIQVITQHQDTAFNMVRTEVRSRVADSHLGHVFPDGPKDRGGLRYCINGGALQFIPVDVMPQSGYAPLVKLVKS

SEQ ID NO: 28 - protein, 552 aa, Brackiella oedipodis
  Methionine sulfoxide reductase, Brackiella oedipodis
MLKQRQLPRFRSLGLSLSVLAVLLGLHSTRVLAAPQSQVAPNSDLRSALGQLTTVTGQSGQNYLRADRPTLVKFWASWCPLCLASLHETSAWSRDQDFAAFNIVSVAAPGYFNELPLEQFKHWFAGVDEADKKGLVVLLNEGGQLTRRLGIAAYPSWALLDRQGRLQRIVKGQLSKEQALGLLTNKDYSLKPAPKSFYKKSSASQQDSATLMNTKTIYLAGGCFWGVEAYFERIPGVVDAVSGYANGKSRHPSYEDVVYRNTGHAETVAVTYDPKQINLAQLLTHYFRLINPTSLNQQGNDRGTQYRTGIYYTDTADKAVISRALADLQHHYKAPIVVENQPLAAFDKAEDYHQDYLAKNPNGYCHIDLRQADQPLSQEELKQVQHIQDATQTDASAPKSPELTPQRFKVPSPEELKKTLSPLAYDVTQNNATERAFTSELDHVFEPGIYVDVVSGEPLFSSTDKFDSGCGWPSFTKPIKADLITEHSDHSYNMIRTEVRSHTANSHLGHVFNDGPKDKGGLRYCINGAALKFIPKDKMQEAGYGDYLQYVK

SEQ ID NO: 29 - protein, 524 aa, Taylorella asinigenitalis
  Methionine sulfoxide reductase, Taylorella asinigenitalis
MGKFLKVLFSVFLVAATQIACSQAKSNSTLSQLKDVDNKSFNIDSSKPTLIKFWASWCPLCLGELPDVENWYKDEAFKGVNLVTIASPSYLSEKKEEAFKNWAKQSGIYKSGSFPIYVDPKGSHAKKWGIKVYPSWVLLDKNQQVQRIIKGSISKKQALALINNKDANLMETEKKYYKESNNGESKIPLRTETIYLAGGCFWGVEAYFQKIPGIVDAVSGYANGNVENPHYRLVTTGTTGFTETVKITYDIDKIGIQEILAHYFRIIDPTSLNKQGNDRGTQYRTGIYYEKPEYKEIVAKALEDLQKKYSEPVVVENMQLKNFYMAEEYHQDYLIKNPNGYCHIDLSLADKPLEGVKKMKKGFDEASYVKPSDEELRKTLTPEQYRVTQEEGTEFAFSHEYDNLFEPGIYVDVVSGEPLFSSDDKYNSGCGWPSFSKPIEDDNIHEKKDFKIGYPRTEVRSSAADSHLGHVFNDGPKELGGLRYCINGASLRFIPYSEMKEQGYEEWMDKVKPIKGGATEVNKK

SEQ ID NO: 30 - protein, 558 aa, Moraxella catarrhalis
  Methionine sulfoxide reductase, Moraxella catarrhalis
MTKPRLRSHACAISLGIFASLSMLSACGKPNDIQTQSVTHQDMLPSDTLAQLSALPQLTQGLGDTGKSVIDPNKPTLVKFWASWCPLCLSTLQETHDWRGDPNLAGFNIITVASPTHLNEKNTQDFTNWYQVLQADYPNLPVLIDSSGQLIKSLGIQVYPSWAILDKNGQLVYLSKGNLSVEQVSYLAKNPQALNELKAQSHQTAMPTKDKDGVHYNDQGMPLNTKTIYLAGGCFWGVEAYFERIDGVVDAVSGYANGDETLKNPSYEQVIAGSGHAETVKVVYDADKMDLDTLLRYYFRIIDPTSVNKQGNDRGIQYRTGVYYTDPSDKAIIDNALNELQQKYKAPIVVENLPLSHFALAEDYHQDYLTKNPNGYCHVDLSLANDKIVSKAQTLPKASTIQEALDPKRYQAFDKDNLKNTLTKAQYDITQNAGTERAFSHAYDHLFDDGLYVDIVSGEPLFLSTDKYNSGCGWPSFTKPIDPQVITEHTDTSYNMVRTEVRSRTADSHLGHVFPDGPKARGGLRYCINGDALKFIPKADMDKHGYGALLPLIKPAQP

SEQ ID NO: 31 - protein, 603 aa, Enhydrobacter aerosaccus
  Methionine sulfoxide reductase, Enhydrobacter aerosaccus
MFLIKNLLECQLTAIFDALQIDEKCTMVNFPQSAKNLTITLLISSLLLLGCQKMNAKENATYAGAASKADVLPTDTLATLQGLSQINPKLGKMGRHVIDPNKPTVVKFWASWCPLCLATLQESDAWAKQYPDMNVISVVSPGHLSEKSSQDFQTWYTVLAKDYANLAVLMDNNGKLIKQFGVQVYPSFAILDKQGNLLKLVKGNLTPTQIQALSDNASNDFAELKALNQAKTPYSQTAQAHEQANNQASKQAASIKALAPINHNGVYYQADGKTPIRTHTIYLAGGCFWGLEAYMERVDGVVDAISGYANGNSANPSYEQVIAGSGHAETVKVIYDIDKTNLATLLAYYVRVIDPTSLNKQGNDRGAQYRTGIYYTDANDKPIIDKTLADLAKKYPQKIVVENKPLANFYDAENYHQDYLSKNPNGYCHIDINLANQKIPVIKSLAPATTVTEALNPSRYQNYDKNVKSRLTQAQYDVTQNAATERAFSHQYDHLFAKGLYVDIVSGEPLFLSTDKYDSGCGWPSFTKPISANVITTSTDSSFNMTRTEVRSRVANSHLGHVFDDGPKDKGGLRYCINGDALQFIALADMQAAGYGALMPLVK

SEQ ID NO: 32 - protein, 496 aa, Fusobacterium mortiferum
  Methionine sulfoxide reductase, Fusobacterium mortiferum
MKKFYKIFLTFLFLIGGTMVFANRRGIENFELKTLDGKEYTLPKGKKVYLKAWASWCPICLSSLEELDSFTKEEDRIEIVTVVFPGKSGEMSKEEFKKWYSSLGYKNIKVLVDEKGELLKKARIRAFPTSIFIDETGEIKGVVPGQLPKEQILKIMGVDSQKKEEVVKKEDNVPVTSKNEGQKIEEIYLAGGCFWGVEAYMERIYGVVDAVSGYANGKTENPRYEDVVYRDTGHAETVKVTYDSNKISLSTLLEYYFRIVDPTSLNKQGNDRGTQYRTGIYYIKAEDEKVVTQALENLQKKYDKKVVIENKPLENFYLAEEYHQDYLKKNPNGYCHIDLNKANDIIVDASKYKKLSDKELREKLSEKEYRITQLNDTERAFDNEYWNFFEPGIYVDITTGEPLFSSKDKYNSMCGWPSFTKPISEDVVTYHTDRSFNMVRTEVRSRVGDAHLGHVFEDGPKDKGGLRYCINSGALNFIPVDEMEKEGYGYLLKLVK

SEQ ID NO: 33 - protein, 379 aa, Helcococcus sueciensis
  Methionine sulfoxide reductase, Helcococcus sueciensis
MKKRFLLIVFAVIFSITACTSKRDVTNSDEKKKDEIRKQIDEIISQHQNENNDENPNDSIDYSKTKLKTIYLAGGCFWGVEAYMEKVYGVADVVSGYANGNTENPTYEDVLYKNTEHAETVKVDYDPEKISLEKILDYYLLVVDPTSLNKQGNDRGTQYRSGVYFTDENERKIIEERLKKEQEKYKDKIVVEVQKLENFYEAEEYHQDYLKKNPNGYCHIDISKANEIIIDQSKYPKPSDEELKKKLTEAQYRVTQENDTEHAFSNEYWDNKEKGIYVDVATGEPLFGSTDKYDSGCGWPSFTKPISKEVVTYHKDFSFNMERTEVRSRSGDSHLGHVFDDGPKESGGLRFCINSASIRFIPLEDMEKEGYGYLTHIIK

SEQ ID NO: 34 - protein, 393 aa, Eremococcus coleocola
  Methionine sulfoxide reductase, Eremococcus coleocola
MKKILLLMVLAATLLVTAYIVKANTTHNELANSEMTNKEMTHNEMKNDDTRNKIDEIITQQQKKSADENPNDAVDYSKAELKTIYLAGGCFWGVEAYLEKVYGVADVVSGYANGDTENPTYEDVSYKNSGHAETVKVDYDPARISLEQILDYYLLVVDPTSMNRQGNDRGLQYRSGVYYTDESERKIIEERLNKEQAKYEDKIVVEVEKLDNFYEAEEYHQDYLKKNPNGYCHIDISKANEVIIDQSKYPKPSDEELKKKLTDVQYKVTQENDTEHAFSNEYWDNKDKGIYVDVATGEPLFSSTDKFDSGCGWPSFSKPIAKEVVTYHTDLSYNMKRTEVRSRSGNSHLGHVFEDGPKELGGLRYCINSASIRFVPLEEMEQEGYGYLTHLIK

SEQ ID NO: 35 - protein, 174 aa, Haloarcula californiae
  Msr-A, accession # WP_049944603.1
MTEQATFAGGCFWCTESVFKQIDGVTDVVSGYAGGHVADPSYEAVCREETGHAECVQLTYDPEEVSYEDLLAVHFTTHTPTTKDREGNDVGTQYRSAVFYHDEAQRETVEALIEEIEPGYDSDIVTEVEPLETFYPAEEYHQDYFEKNPDQSYCQLTIPPKIEKLKQKHAELLA

SEQ ID NO: 36 - protein, 179 aa, Halococcus salifodinae
  Msr-A, accession # WP_005043086.1
MATETERATLAGGCFWCIEAPMEELDGVHDVTSGYAGGHTENPTYRAVCSGDTGHAEVVQIEYDPDRIAYEDLLDVLFTVHDPTQLNRQGPDVGTQYRSAIFTHDESQHETAAAYIDALDAEGGYDDPVVTEIEPLETFYEASEEHQNYYEKNPEDAYCSFHAQPKIEKVREKFAEKTA

SEQ ID NO: 37 - protein, 177 aa, Haloferax sp. SB29
  Msr-A, accession # WP_058572480.1
MESSQTATFGGGCFWCIEAAFKELDGISDVTSGYAGGTVENPTYEQVCSGTTGHAEVIQVEYDPSVVDYDELLDVFFAVHDPTQLNRQGPDVGTQYRSIVLYHDDDQRRLAEAYVEALDDSYDDDVVTELAPFETFYEAEAYHQDYFEKNPNDAYCQFHASPKIEKVREKFADKLAN

SEQ ID NO: 38 - protein, 177 aa, Natronococcus occultus
  Msr-A, accession # WP_015322392.1
MERATFGGGCFWCVEAAFEQLEGVDSVTSGYAGGHTEDPTYEAVCSGSTGHAEVVQVEYNPDEIAYEDLLEVFFTVHDPTTKDREGPDVGSQYRSAIYAHDEAQLETAEAFADELEAEGLYEGIVTEIEPLDTFYEAEQYHQNYFEKNPNDAYCSMHAAPKVETVREKFGENVAPEH

SEQ ID NO: 39 - protein, 182 aa, Natronomonas moolapensis
  Msr-A, accession # WP_015408133.1
MSDASHDDELETATLGGGCFWCVEAVLKELDGVRSVTSGYAGGHVEDPSYEAVCRGETGHAEVVQVAFAPETIAFRDLLEVFFTIHTPTTLNREGPDVGSQYRSAVYYHNDEQRRVVESVIGELEPLYDDDIVTEVEPLETFYPAEEYHQDYFDKNPSDTYCTVNVNPKLSKLREKHAELLA

SEQ ID NO: 40 - protein, 176 aa, Natrinema versiforme
  Msr-A, accession # WP_006431385.1
MERATFGGGCFWCTEAAMKELEGVDSVTSGYAGGHTEDPSYREVCSGNTGHAEVVQVEYDPDAIGYDELLEVFFATHDPTQLNRQGPDVGTQYRSIVLYHDDDQRTQAEAYIDALDSEYDDDVVTELEPLETFYRAEEKHQDYFEKNPNDAYCTMHAAPKVEKVREKFAENVAAEH

SEQ ID NO: 41 - protein, 134 aa, Haloarcula sinaiiensis
  Msr-B, accession # WP_004963222.1
MSESEEELPDKDEEWREILSDEEYRILRESGTEPRFSSDLIDVEDEGVFTCAGCGTELFDSDRKFESETGWPSFWDVYQEGNVETRADNSHGMERTEVICAECGGHLGHVFDDGPEPSGKRYCINGAALDFESE

SEQ ID NO: 42 - protein, 139 aa, Halococcus sediminicola
  Msr-B, accession # WP_049996544.1
MSNEPATTGELPETDEEWREVLTDEEYEILREQGTEPKFSGELLDQHDDGTFVCAGCGTELFSSDTKFESKTGWPSFSDVADEGNVELRRDTSHGMERTEVVCATCGGHLGHVFDDGPEPTGKRYCINSAALGFDGDES

SEQ ID NO: 43 - protein, 133 aa, Haloferax sulfurifontis
  Msr-B, accession # WP_007275637.1
MSDSEFSLSESEWRERLSEDAYRVLREQGTEPRFSGEHVDRSDDGVYRCAGCGTELFDSETKYDSNCGWPSFYAAEDSNIELRRDLSHGMDRTEVVCSTCGGHLGHVFDDGPEPTGKRFCINSAALDFEADEE

SEQ ID NO: 44 - protein, 132 aa, Natronococcus jeotgali
  Msr-B, accession # WP_008423757.1
MSNEPDVPTDDQEWREELTDEQYRILREAGTEAPFSGEYVDHKDDGSYACVGCGTTLFDSETKFDSGCGWPSFSDVDDDRVETRLDTSHGMRRTEVLCANCGGHLGHVFDDGPEPTGKRYCINSAVLEFDGE

SEQ ID NO: 45 - protein, 131 aa, Natronomonas moolapensis
  Msr-B, accession # WP_015408129.1
MDSKLPQTDAEWREVLTDEEYRILREQGTEPKFSGEHLGADADGVYRCAGCGAELFDSETKFDSNSGWPSFYDAEEGAVELREDRSHGMVRTEVVCARCEGHLGHVFEDGPDPTGQRYCMNSVALEFDDEA

SEQ ID NO: 46 - protein, 132 aa, Natrinema altunense
  Msr-B, accession # WP_007109050.1
MSDESDHVPTNDEEWRERLSDEEYRILREAGTETPFSGEYVDHKADGSYACAGCGAELFDSETKFDSGCGWPSFYDADDDRIETRTDTSHGMRRTEVVCANCGGHLGHVFDDGPEPTGKRYCINSVALEFDE

SEQ ID NO: 47 - protein, 147 aa, Candidatus Halobonum tyrrellensis
  Msr-B, accession # WP_023395429.1
MSETDETPTDERRSDESLPETDDEWRERLSDEEYEILRERGTEARFSGEHVDRDDDGVYECAGCGTVIFDSGTKYDSGCGWPSFYAADDSKVTLRDDDRHGMSRVEVLCANCDGHLGHVFQDGPEPTGERFCINSVALDFESRERAD

SEQ ID NO: 48 - protein, 19 aa, Artificial Sequence
  Description of Artificial Sequence: Synthetic peptide
EIINVGHSFHVNFEDNDNR

(19) United States
(12) Patent Application Publication
(10) Pub. No.: US 2019/0072565 A1
(43) Pub. Date: Mar. 7, 2019

(54) PROTEIN PREPARATION

(71) Applicants: Pierce Biotechnology, Inc., Rockford, IL (US); Thermo Fisher Scientific Baltics UAB, Vilnius (LT)

(72) Inventors: John Rogers, Rockford, IL (US); Robert Cunningham, Rockford, IL (US); Juozas Siurkus, Vilnius (LT); Bhavinkumar Patel, Rockford, IL (US)

(21) Appl. No.: 16/070,225

(22) PCT Filed: Jan. 4, 2017
(86) PCT No.: PCT/US2017/012121

§ 371 Date: Jul. 13, 2018

Related U.S. Application Data
(60) Provisional application No. 62/368,336, filed on Jul. 29, 2016.
(60) Provisional application No. 62/279,421, filed on Jan. 15, 2016.

(51) Int. Cl. G01N 33/68; C12N 15/70; C12P 21/02; C12N 9/02

(52) U.S. Cl. CPC G01N 33/6848; C12N 15/70; C12P 21/02; C12N 9/0051; G01N 2560/00; C12Y 108/01009; C12Y 108/04013; C12Y 108/04014; C07K 2319/00; C12Y 108/01008

(57) ABSTRACT

This disclosure relates to the field of protein preparation and mass spectrometry analysis. In some embodiments, the disclosure relates to compositions and methods for simplifying mass spectrometry analysis by reducing methionine oxidation of protein samples.

This application is a 371 National Stage filing of PCT/US2017/012121, filed Jan. 4, 2017, which claims the benefit of priority of U.S. Provisional Application No. 62/279,421, filed Jan. 15, 2016, and U.S. Provisional Application No. 62/368,336, filed Jul. 29, 2016, each of which is incorporated by reference herein in its entirety for any purpose.

This disclosure relates to the field of protein preparation, for example, protein preparation prior to liquid chromatography and/or mass spectrometry analysis.

Methionine (Met) is a sulfur-containing essential amino acid that is highly susceptible to oxidation by reactive oxygen species (ROS) during stress, aging, and disease in vivo. Met residues are believed to act as a scavenger for reactive oxygen species, minimizing oxidative damage to other amino acids and biomolecules in the cell and protecting active site residues from oxidation. Met oxidation can also affect protein structure and function and can serve as a biological switch that is sensitive to oxidative stress. Methionine oxidation is also commonly observed during protein purification and analysis in vitro. The product of Met oxidation is Met sulfoxide (MetO), which exists in the form of two diastereomers, methionine S-sulfoxide and methionine R-sulfoxide.

Methionine oxidation can occur during protein sample preparation and purification. The release of peroxisomal contents during tissue and cellular lysis, the exposure of protein-containing solutions during sample processing to air, ionizing light and radiation, and exposure to metals that produce free radicals via the Fenton reaction can all cause methionine oxidation during preparation or storage. Variable methionine oxidation of purified proteins, including biotherapeutic proteins, leads to lower and variable product quality, and the ability to reverse or prevent methionine oxidation would benefit protein therapeutics.

Some of this oxidation can be prevented by free radical scavengers (e.g. glycerols) or metal chelators (e.g. EDTA), but some of the oxidation may be introduced naturally in the cell prior to any processing. The reversal of methionine oxidation, whether introduced in vivo or in vitro, with chemical treatment has been described, but this requires extreme pH conditions that may affect proteins in undesirable ways (e.g. precipitation, deamidation, or other modifications). Complete oxidation of methionine to sulfone has also been attempted, but the side reactions from over-oxidation at cysteine, tryptophan, and other amino acids produced undesirable results.

Methionine oxidation is commonly observed by mass spectrometry. Oxidation of methionines and other amino acids typically manifests itself as a series of 15.995 Da mass increases from the original unmodified mass spectral peak of the proteoforms of interest. The stochastic nature of this variable oxidation may interfere with protein liquid chromatography and/or mass spectrometry analysis in at least four ways: 1) poor peak shape and resolution during liquid chromatography; 2) increased complexity and poorer depth of analysis of intact proteins due to multiple oxidized proteoforms; 3) increased complexity and poorer depth of analysis of peptides from protein digests due to multiple oxidized forms, and; 4) impaired quantitation of specific methionine-containing peptides due to reduced sensitivity and variability in the stoichiometry of oxidized peptides.

Further, the increased hydrophilicity of a protein caused by methionine oxidation affects retention and chromatography. This results in peaks for each oxidized form of a protein, less intense peaks from each protein form due to dilution of the protein forms across multiple peaks, and greater variability in peak areas because of random, incomplete, and variable stoichiometry of oxidation at each methionine in a sequence. In addition, the diasteromers of methionine sulfoxide in a protein or peptide may be resolved or spread into overlapping peaks with high performance liquid chromatography. This peak broadening and poor resolution further affects sensitivity, peak integration, and quantitation. The reversal of methionine oxidation can improve protein chromatography by improving sample purity and chromatographic behavior.

Mass spectrometry is used to characterize intact proteins and complexes at the molecular level. While the analysis of intact proteins can provide important insights, the isolation and fragmentation of these proteins in the mass spectrometer by collisional and non-ergodic approaches provides information about the complete sequence and sites of modification of a protein. This intact protein MS/MS approach is often referred to as “top-down” or “middle-down” protein analysis. Methionine oxidation increases sample complexity and severely impairs top-down protein analysis because it increases sample complexity and complicates the data analysis. For each methionine in a protein (N), the number of proteoforms caused by variable oxidation of each methionine to the sulfoxide form may be up to the factorial of N.

Oxidation of proteins during electrospray ionization has been known for over 20 years. Methionine oxidation has been shown to occur during the electrospray ionization of proteins for MS analysis. This oxidation can be limited or prevented by using polished metal surfaces and by modifying the liquid chromatography or electrochemical junction to avoid direct application of high voltage to protein containing solution.

The present disclosure provides methods of reducing or eliminating methionine oxidation in protein samples prior to separation and/or analysis, such as, for example, liquid chromatography and/or mass spectrometry analysis.

In some embodiments, methods of preparing a polypeptide sample for separation and/or analysis are provided, comprising contacting the polypeptide sample with at least one methionine sulfoxide: reductase enzyme under conditions suitable for reducing oxidized methionines in the polypeptide sample. In some embodiments, at least one methionine sulfoxide reductase enzyme is capable of reducing methionine-S-sulfoxide, or is capable of reducing methionine-R-sulfoxide, or is capable of reducing both methionine-S-sulfoxide and methionine-R-sulfoxide. In some embodiments, the method comprises contacting the polypeptide sample with at least one methionine sulfoxide reductase enzyme that is capable of reducing methionine-S-sulfoxide and at least one methionine sulfoxide reductase enzyme that is capable of reducing methionine-R-sulfoxide. In some embodiments, the method comprises contacting the polypeptide sample with an MsrA enzyme and an MsrB enzyme. In some embodiments, an MsrA is derived from an MsrA enzyme of an organism selected from Haloarcula, Halococcus, Haloferax, Natronococcus, Natronomonas, and Natrinema. In some embodiments, an MsrA is at least 70%, at least 80%, or at least 90%, at least 91%, at least 92%, at least 93%, at least 94%, at least 95%, at least 96%, at least 97%, at least 98%, at least 99%, or 100% identical to an MsrA enzyme under accession number WP_049944603.1, WP_005043086.1, WP_058572480.1, WP_015322392.1, WP_015408133.1, and WP_006431385.1. In some embodiments, an MsrB is derived from an MsrB enzyme of an organism selected from Haloarcula, Halococcus, Haloferax, Natronococcus, Natronomonas, Natrinema, and Candidatus Halobonum. In some embodiments, an MsrB is at least 70%, at least 80%, or at least 90%, at least 91%, at least 92%, at least 93%, at least 94%, at least 95%, at least 96%, at least 97%, at least 98%, at least 99%, or 100% identical to an MsrA enzyme under accession number WP_004963222.1, WP_049996544.1, WP_007275637.1, WP_008423757.1, WP_015408129.1, WP_007109050.1, and WP_023395429.1. In various embodiments, an MsrA and MsrB may be from the same or different organism.

In some embodiments, the method comprises contacting the polypeptide sample with at least one methionine sulfoxide reductase enzyme that is capable of reducing both methionine-S-sulfoxide and methionine-R-sulfoxide. In some embodiments, the method comprises contacting the polypeptide sample with an MsrAB enzyme. In some embodiments, the MsrAB enzyme is derived from a methionine sulfoxide reductase from an organism selected from Neisseria, Lautropia, Cardiobacterium, Gammaproteobacteria, Pelistega, Marinospirillum, Basilea, Oligella, Alcagenaceae, Psychrobacter, Brackiella, Taylorella, Moraxella, Enhydrobacter, Fusobacterium, Helcococcus, Paenibacillus, Eremococcus, Methanobrevibacter, Methanomassiliicoccales, Methanocorpusculum, Thermoplasmatales, Methanometylophilus, Methanoculleus, and Methanocella. In some embodiments, the MsrAB enzyme is derived from a bacterial methionine sulfoxide reductase enzyme. In some embodiments, the methionine sulfoxide reductase enzyme is derived from a methionine sulfoxide reductase enzyme of Neisseria gonorrhoeae, Neisseria meningitides, Neisseria lactamica, Neisseria polysaccharea, Neisseria flavescens, Neisseria sicca, Neisseria macacacae, or Neisseria mucosa. In some embodiments, the methionine sulfoxide reductase enzyme comprises an amino acid sequence that is at least 70%, at least 80%, at least 90%, at least 91%, at least 92%, at least 93%, at least 94%, at least 95%, at least 96%, at least 97%, at least 98%, at least 99%, or 100% identical to a sequence selected from SEQ ID NOs: 10 to 34.

In some embodiments, the methionine sulfoxide reductase enzyme is bound to a solid support. In some embodiments, the methionine sulfoxide reductase enzyme is bound to a resin or a bead. In some embodiments, the method further comprises removing the methionine sulfoxide reductase enzyme following reduction of oxidized methionines in the polypeptide sample.

In some embodiments, the methionine sulfoxide reductase enzyme is present at a weight ratio of between 1:100 and 1:2 enzyme:polypeptide. In some embodiments, the contacting occurs under reducing conditions. In some embodiments, the contacting occurs in the presence of dithiothreitol (DTT) and/or dithioerythritol (DTE). In some embodiments, the method comprises preparing a sample for liquid chromatography. In some embodiments, the method further comprises subjecting polypeptides of the polypeptide sample to liquid chromatography. In some embodiments, the liquid chromatography is high performance liquid chromatography.

In some embodiments, the method comprises preparing a sample for capillary electrophoresis. In some embodiments, the method further comprises subjecting polypeptides of the polypeptide sample to capillary electrophoresis.

In some embodiments, the method comprises preparing a sample for mass spectrometry. In some embodiments, the method further comprises subjecting polypeptides of the polypeptide sample to mass spectrometry.

In some embodiments, the method comprises fragmenting the polypeptides of the polypeptide sample. In some embodiments, the method comprises fragmenting the polypeptides by proteolytic or chemical cleavage. In some embodiments, the fragmented polypeptides are peptides consisting of 5 to 50 amino acids. In some embodiments, the method comprises fragmenting the polypeptides by digestion with trypsin, chymotrypsin, AspN, GluC, LysC, LysN, ArgC, proteinase K, or thermolysin, or by chemical cleavage with CNBr. In some embodiments, the method comprises separating the polypeptides on a gel and then fragmenting the polypeptides in gel.

In some embodiments, the polypeptides of the polypeptide sample were previously fragmented. In some embodiments, the polypeptides were fragmented by proteolytic or chemical cleavage. In some embodiments, the polypeptide fragments are peptides consisting of 5 to 50 amino acids. In some embodiments, the polypeptide fragments were produced by digestion with trypsin, chymotrypsin, AspN, GluC, LysC, LysN, ArgC, proteinase K, or thermolysin, or by chemical cleavage with CNBr. In some embodiments, the polypeptide fragments were produced in solution or in gel following gel separation of the protein. In some embodiments, the method comprises separating the polypeptides or polypeptide fragments from other components of the polypeptide sample. In some embodiments, the method comprises subjecting the polypeptides or polypeptide fragments to mass spectrometry analysis. In some embodiments, the mass spectrometry analysis comprises internal fragmentation of the polypeptides or polypeptide fragments.

In some embodiments, methods of producing a mass spectrometry spectrum are provided, comprising contacting polypeptides of a polypeptide sample with at least one methionine sulfoxide reductase enzyme under conditions suitable for reducing oxidized methionines in the polypeptide sample, and injecting the polypeptides into a liquid chromatograph/mass spectrometer detection system or directly into a mass spectrometer detection system, wherein the polypeptides of the polypeptide sample have not been fragmented. In some embodiments, at least one methionine sulfoxide reductase enzyme is capable of reducing methionine-S-sulfoxide, or is capable of reducing methionine-R-sulfoxide, or is capable of reducing both methionine-S-sulfoxide and methionine-R-sulfoxide. In some embodiments, the method comprises contacting the polypeptide sample with at least one methionine sulfoxide reductase enzyme that is capable of reducing methionine-S-sulfoxide and at least one methionine sulfoxide reductase enzyme that is capable of reducing methionine-R-sulfoxide. In some embodiments, the method comprises contacting the polypeptide sample with an MsrA enzyme and an MsrB enzyme. In some embodiments, an MsrA is derived from an MsrA enzyme of an organism selected from Haloarcula, Halococcus, Haloferax, Natronococcus, Natronomonas, and Natrinema. In some embodiments, an MsrA is at least 70%, at least 80%, or at least 90%, at least 91%, at least 92%, at least 93%, at least 94%, at least 95%, at least 96%, at least 97%, at least 98%, at least 99%, or 100% identical to an MsrA enzyme under accession number WP_049944603.1, WP_005043086.1, WP_058572480.1, WP_015322392.1, WP_015408133.1, and WP_006431385.1. In some embodiments, an MsrB is derived from an MsrB enzyme of an organism selected from Haloarcula, Halococcus, Haloferax, Natronococcus, Natronomonas, Natrinema, and Candidatus Halobonum. In some embodiments, an MsrB is at least 70%, at least 80%, or at least 90%, at least 91%, at least 92%, at least 93%, at least 94%, at least 95%, at least 96%, at least 97%, at least 98%, at least 99%, or 100% identical to an MsrA enzyme under accession number WP_004963222.1, WP_049996544.1, WP_007275637.1, WP_008423757.1, WP_015408129.1, WP_007109050.1, and WP_023395429.1. In various embodiments, an MsrA and MsrB may be from the same or different organism.

In some embodiments, the method comprises contacting the polypeptide sample with at least one methionine sulfoxide reductase enzyme that is capable of reducing both methionine-S-sulfoxide and methionine-R-sulfoxide. In some embodiments, the method comprises contacting the polypeptide sample with an MsrAB enzyme. In some embodiments, the MsrAB enzyme is derived from a methionine sulfoxide reductase from an organism selected from Neisseria, Lautropia, Cardiobacterium, Gammaproteobacteria, Pelistega, Marinospirillum, Basilea, Oligella, Alcagenaceae, Psychrobacter, Brackiella, Taylorella, Moraxella, Enhydrobacter, Fusobacterium, Helcococcus, Paenibacillus, Eremococcus, Methanobrevibacter, Methanomassiliicoccales, Methanocorpusculum, Thermoplasmatales, Methanometylophilus, Methanoculleus, and Methanocella. In some embodiments, the MsrAB enzyme is derived from a bacterial methionine sulfoxide reductase enzyme. In some embodiments, the methionine sulfoxide reductase enzyme is derived from a Neisseria methionine sulfoxide reductase enzyme. In some embodiments, the methionine sulfoxide reductase enzyme is derived from a methionine sulfoxide reductase enzyme of Neisseria gonorrhoeae, Neisseria meningitides, Neisseria lactamica, Neisseria polysaccharea, Neisseria flavescens, Neisseria sicca, Neisseria macacae, or Neisseria mucosa. In some embodiments, the methionine sulfoxide reductase enzyme comprises an amino acid sequence that is at least 70%, at least 80%, at least 90%, at least 91%, at least 92%, at least 93%, at least 94%, at least 95%, at least 96%, at least 97%, at least 98%, at least 99%, or 100% identical to a sequence selected from SEQ ID NOs: 10 to 34.

In some embodiments, the methionine sulfoxide reductase enzyme is bound to a solid support. In some embodiments, the methionine sulfoxide reductase enzyme is bound to a resin or a bead. In some embodiments, the method further comprises removing the methionine sulfoxide reductase enzyme following reduction of oxidized methionines in the polypeptide sample.

In some embodiments, each methionine sulfoxide reductase enzyme is present at a weight ratio of between 1:100 and 1:2 enzyme:polypeptide. In some embodiments, the contacting occurs under reducing conditions. In some embodiments, the contacting occurs in the presence of dithiothreitol (DTT) or dithioerythritol (DTE).

In some embodiments, kits comprising at least one methionine sulfoxide reductase enzyme are provided. In some embodiments, a kit comprises at least one reagent for fragmenting a polypeptide sample for mass spectrometry analysis. In some embodiments, the kit comprises at least one reagent selected from trypsin, chymotrypsin, AspN, GluC, LysC, LysN, ArgC, proteinase K, thermolysin, and CNBr. In some embodiments, the at least one methionine sulfoxide reductase enzyme is capable of reducing methionine-S-sulfoxide, or is capable of reducing methionine-R-sulfoxide, or is capable of reducing both methionine-S-sulfoxide and methionine-R-sulfoxide. In some embodiments, the kit comprises at least one methionine sulfoxide reductase enzyme that is capable of reducing methionine-S-sulfoxide and at least one methionine sulfoxide reductase enzyme that is capable of reducing methionine-R-sulfoxide. In some embodiments, the kit comprises an MsrA enzyme and an MsrB enzyme. In some embodiments, an MsrA is derived from an MsrA enzyme of an organism selected from Haloarcula, Halococcus, Haloferax, Natronococcus, Natronomonas, and Natrinema. In some embodiments, an MsrA is at least 70%, at least 80%, or at least 90%, at least 91%, at least 92%, at least 93%, at least 94%, at least 95%, at least 96%, at least 97%, at least 98%, at least 99%, or 100% identical to an MsrA enzyme under accession number WP_049944603.1, WP_005043086.1, WP_058572480.1, WP_015322392.1, WP_015408133.1, and WP_006431385.1. In some embodiments, an MsrB is derived from an MsrB enzyme of an organism selected from Haloarcula, Halococcus, Haloferax, Natronococcus, Natronomonas, Natrinema, and Candidatus Halobonum. In some embodiments, an MsrB is at least 70%, at least 80%, or at least 90%, at least 91%, at least 92%, at least 93%, at least 94%, at least 95%, at least 96%, at least 97%, at least 98%, at least 99%, or 100% identical to an MsrA enzyme under accession number WP_004963222.1, WP_049996544.1, WP_007275637.1, WP_008423757.1, WP_015408129.1, WP_007109050.1, and WP_023395429.1. In various embodiments, an MsrA and MsrB may be from the same or different organism.

In some embodiments, the kit comprises at least one methionine sulfoxide reductase enzyme that is capable of reducing both methionine-S-sulfoxide and methionine-R-sulfoxide. In some embodiments, the kit comprises an MsrAB enzyme. In some embodiments, the MsrAB enzyme is derived from a methionine sulfoxide reductase from an organism selected from Neisseria, Lautropia, Cardiobacterium, Gammaproteobacteria, Pelistega, Marinospirillum, Basilea, Oligella, Alcagenaceae, Psychrobacter, Brackiella, Taylorella, Moraxella, Enhydrobacter, Fusobacterium, Helcococcus, Paenibacillus, Eremococcus, Methanobrevibacter, Methanomassiliicoccales, Methanocorpusculum, Thermoplasmatales, Methanometylophilus, Methanoculleus, and Methanocella. In some embodiments, the MsrAB enzyme is derived from a bacterial methionine sulfoxide reductase enzyme. In some embodiments, the methionine sulfoxide reductase enzyme is derived from a methionine sulfoxide reductase enzyme of Neisseria gonorrhoeae, Neisseria meningitides, Neisseria lactamica, Neisseria polysaccharea, Neisseria flavescens, Neisseria sicca, Neisseria macacacae, or Neisseria mucosa. In some embodiments, the methionine sulfoxide reductase enzyme comprises an amino acid sequence that is at least 70%, at least 80%, at least 90%, at least 91%, at least 92%, at least 93%, at least 94%, at least 95%, at least 96%, at least 97%, at least 98%, at least 99%, or 100% identical to a sequence selected from SEQ ID NOs: 10 to 34.

In some embodiments, a kit comprises at least one methionine sulfoxide reductase enzyme bound to a solid support. In some embodiments, the methionine sulfoxide reductase enzyme is bound to a resin or a bead.

In some embodiments, methods of reducing the complexity of a protein sample are provided. In some embodiments, methods of reducing the complexity of a protein sample prior to analysis by mass spectrometry (MS) are provided. In some embodiments, oxidized methionine stereoisomers are reduced by methionine sulfoxide reductase A and methionine sulfoxide reductase B (MsrA and MsrB) to restore methionine sulfoxide residues back to native methionine amino acids. See, e.g., FIG. 1. In some embodiments, this reduces sample variability and complexity by consolidating multiple versions of a methionine-containing protein into one species, which in some instances permits more efficient and sensitive MS acquisition and faster data analysis. In various embodiments, the sample may be a purified or enriched protein sample, or may be a complex biological sample. The purpose of analysis may, in various embodiments, be identification, characterization, and/or quantitation of a protein. Further, in various embodiments, the reduction of methionine sulfoxide to methionine may be performed before, during, and/or after the reduction of cysteines and cysteine disulfides and/or fragmentation of the protein. In various embodiments, the methods are used in a diagnostic assay. In some embodiments, the methods are used during multi-sample analysis and/or multi-target analysis.

In some embodiments, methods for protein or peptide quantitation by mass spectrometry are provided. In some such embodiments, a method comprises (a) preparing a sample containing a target protein or peptide of interest for mass spectrometry analysis, (b) adding an isotope-labeled peptide or protein (such as a heavy, stable isotope-labeled peptide or protein) having at least one subsequence of the target protein or peptide containing methionine, (c) mixing the isotope labeled peptides or proteins at known concentrations with the sample, (d) treating the sample with a methionine sulfoxide reductase (such as MsrA/B) and reagents suitable for MsrA/B activity, to reduce methionine sulfoxide residues to methionine, (e) subjecting the mixture containing the prepared sample and the isotope-labeled proteins or peptides to mass spectrometry analysis. In some embodiments, a single additive heavy peptide mass spectrometry peak is obtained with the corresponding single light peptide (i.e., non-isotope-labeled peptide) mass spectrometry peak. In some embodiments, the method further comprises (f) subjecting the mass peaks to isolation and fragmentation by mass spectrometry analysis. In some embodiments, unique and confirming mass spectrometry peaks are obtained representing the protein or peptide fragments of the light and isotope-labeled (such as heavy isotope labeled) proteins or peptides. In some embodiments, the method further comprises (g) generating a light peptide intensity: heavy peptide intensity ratio, and (h) quantifying the intensity of each of the plurality of mass spectrometry peaks based on the intensity of the heavy isotope labeled peptides, e.g., to quantify the amount of protein or peptide in the sample. In some embodiments, the isotope-labeled peptides and proteins are prepared by synthesizing the peptides or proteins in vitro or in vivo with amino acid precursors that contain isotopes or oxidized methionine, resulting in isotope-labeled peptides and proteins that may contain native or oxidized methionine.

In some embodiments, a method of using a methionine sulfoxide reductase (Msr) protein sequence to monitor the consistency and completeness of fragmentation (such as digestion) of a protein sample is provided. For example, a known amount of soluble Msr enzyme may be added to a sample to reverse methionine oxidation prior to proteolytic digestion, and the known and unique peptides from the Msr enzyme may be monitored to assess the efficiency of fragmentation and recovery of peptides.

In some embodiments, methods of reversing protein methionine oxidation are provided, which use an Msr enzyme containing an affinity tag or Msr enzyme immobilized on a resin or bead. In some such embodiments, a protein sample may have an unknown or undesired level of methionine oxidation, but it may be undesirable to contaminate the sample with the Msr protein. In some such embodiments, the Msr enzyme is added to the protein sample under conditions such that methionine sulfoxide can be reversed, and the Msr enzyme can then be efficiently removed by addition of an affinity resin to capture the tagged Msr enzyme, and the resin with captured or immobilized Msr can be removed by centrifugation or filtration.

In some embodiments, methods of reducing the complexity of intact protein samples prior to MS/MS analysis by mass spectrometry are provided. The use of high resolution MS and multiple fragmentation methods to determine the complete structure of a protein may be referred to as “top-down” proteomics. The stochastic nature of methionine oxidation may result in multiple isobaric and non-isobaric variants of a protein, resulting in increased sample complexity and difficulty interpreting the protein fragments from co-isolated, isobaric protein species containing oxidized methionine at different locations. In some embodiments, the reduction of methionine sulfoxides by Msr reduces the complexity of the intact protein(s) and the fragmentation products while increasing the signal to noise ratio.

In some embodiments, methods of reducing methionine oxidation prior to liquid chromatography or other analysis or purification methods are also provided.

### BRIEF DESCRIPTION OF THE FIGURES

FIG. 1 shows structures of oxidized methionine and reduction by methionine sulfoxide reductases. ROS is “reactive oxygen species.”

FIG. 2 shows intact protein MS analysis of methionine oxidation of TurboLuc in the absence of peroxide treatment. Un-oxidized samples or samples treated with the Msrs ngMsrAB or nmMsrAB are presented.

FIG. 3 shows intact protein MS analysis of methionine oxidation of TurboLuc following 10× peroxide treatment. Samples were oxidized alone or oxidized and then treated with the Msrs ngMsrAB or nmMsrAB.

FIG. 4 shows intact protein MS analysis of methionine oxidation of TurboLuc following 25× peroxide treatment. Samples were oxidized alone or oxidized and then treated with the Msrs ngMsrAB or nmMsrAB.

FIG. 5 shows intact protein MS analysis of methionine oxidation of TurboLuc following 75× peroxide treatment. Samples were oxidized alone or oxidized and then treated with the Msrs ngMsrAB or nmMsrAB.

FIG. 6 shows intact protein MS analysis of methionine oxidation of TurboLuc following 100× peroxide treatment. Samples were oxidized alone or oxidized and then treated with the Msrs ngMsrAB or nmMsrAB.

FIG. 7 shows intact protein MS analysis of methionine oxidation of TurboLuc following 500× peroxide treatment. Samples were oxidized alone or oxidized and then treated with the Msrs ngMsrAB or nmMsrAB.

FIG. 8 outlines an exemplary procedure for shotgun proteomic analysis.

FIG. 9 shows shotgun proteomic analysis results on methionine oxidation of a 6-protein sample. Reversal of methionine oxidation was measured for samples treated with ngMsrAB or nmMsrAB. The percentage of oxidized methionine and doubly oxidized methionine are presented for control (no oxidation), 1:25, 1:50, and 1:100 methionine:peroxide ratios. Met, methionine.

FIGS. 10A and 10B present the protocol (A) and actual readout (B) of parallel reaction monitoring (PRM) of a representative experiment.

FIGS. 11A and 11B present the effect of treatment with nmMsrAB and ngMsrAB on the disappearance of an oxidized peptide standard (SEQ ID No: 4) and appearance of the corresponding reduced peptide (SEQ ID No: 5) using PRM analysis.

FIGS. 12A and 12B present the effect of treatment with nmMsrAB and ngMsrAB on the disappearance of an oxidized phosphopeptide (SEQ ID No: 6) and appearance of the corresponding reduced peptide (SEQ ID No: 7) using PRM analysis.

FIGS. 13A and 13B present the effect of treatment with nmMsrAB and ngMsrAB on the disappearance of another oxidized phosphopeptide (SEQ ID No: 8) and corresponding appearance of a reduced phosphopeptide (SEQ ID No: 9) using PRM analysis.

FIG. 14 shows the sequence of His-tagged/WQ MsrAB from Neisseria gonorrhoeae (SEQ ID NO: 1). The His tag/WQ protease site is shown in italics. A coommassie-stained gel showing the predominant expressed species is also shown. The terms “ngMsrAB” and “ngMsrAB-T” are used interchangeably.

FIG. 15 shows the sequence of His-tagged/WQ MsrAB from Neisseria meningitidis (SEQ ID NO: 2). The His tag/WQ protease site is shown in italics. A coommassie-stained gel showing the predominant expressed species is also shown. The terms “nmMsrAB” and “nmMsrAB-T” are used interchangeably.

FIG. 16 shows how the theoretical reversal of methionine oxidation results in a less convoluted spectrum in “top down” protein MS/MS analysis.

### DETAILED DESCRIPTION

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

As used herein, the terms “methionine sulfoxide reductase”, “Msr”, “MetSR”, and “Msr enzyme” are used interchangeably to refer to a methionine sulfoxide reductase that is capable of reducing methionine-S-sulfoxide and/or methionine-R-sulfoxide. A Msr domain that is capable of reducing methionine-S-sulfoxide to methionine is referred to as an “A domain.” A Msr domain that is capable of reducing methionine-R-sulfoxide to methionine is referred to as an “B domain.” Thus, the terms “methionine sulfoxide reductase”, “Msr”, “MetSR”, and “Msr enzyme” refer generically to a methionine sulfoxide reductase enzyme that comprises a methionine sulfoxide reductase A domain alone, B domain alone, or both an A domain and a B domain. In some embodiments, a Msr is a MsrAB. In some embodiments, a Msr is a MsrA. In some embodiments, a Msr is a MsrB.

As used herein, the terms “methionine sulfoxide reductase AB”, “MsrAB”, “MetSR-AB”, and “MsrAB enzyme” are used interchangeably to refer to a methionine sulfoxide reductase comprising a methionine sulfoxide reductase A domain and a methionine sulfoxide reductase B domain, wherein the reductase is capable of reducing both methionine-S-sulfoxide and methionine-R-sulfoxide. In some embodiments, the MsrAB enzyme comprises a thioredoxin (Trx) domain. In some such embodiments, the MsrAB enzyme may be referred to as a MsrAB-T enzyme.

The terms “methionine sulfoxide reductase A”, “MsrA”, “MetSR-A, and “MsrA enzyme” are used interchangeably to refer to a methionine sulfoxide reductase comprising a methionine sulfoxide reductase A domain, wherein the reductase is capable of reducing methionine-S-sulfoxide.

The terms “methionine sulfoxide reductase B”, “MsrB”, “MetSR-B”, and “MsrB enzyme” are used interchangeably to refer to a methionine sulfoxide reductase comprising a methionine sulfoxide reductase A domain, wherein the reductase is capable of reducing methionine-R-sulfoxide.

As used herein “protein”, “peptide”, and “polypeptide” are used interchangeably throughout to mean a chain of amino acids wherein each amino acid is connected to the next by a peptide bond. In some embodiments, when a chain of amino acids consists of about two to fifty amino acids, the term “peptide” is used. However, the term “peptide” should not be considered limiting unless expressly indicated.

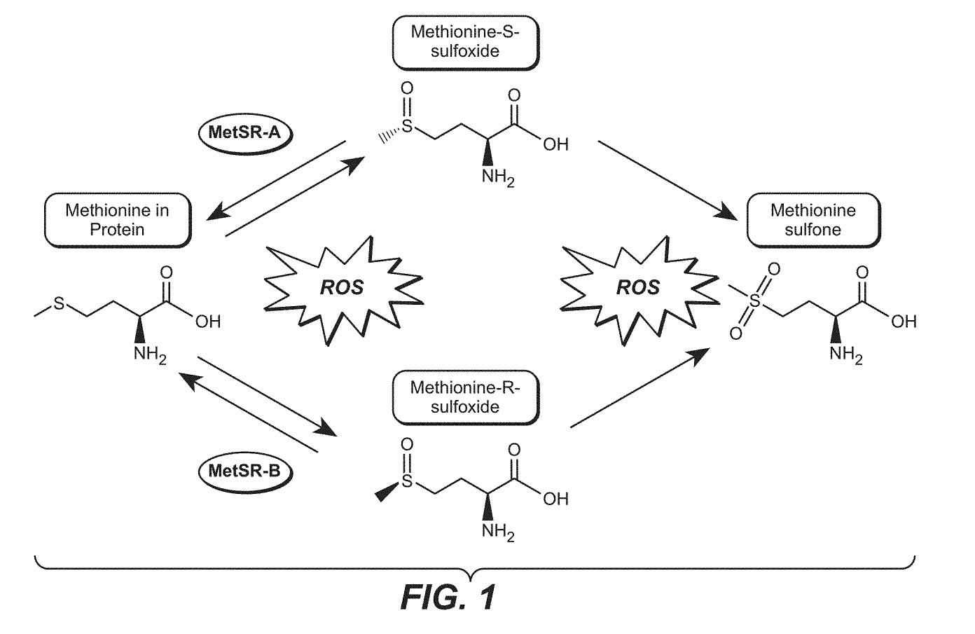

In cells, methionine sulfoxides are reduced back to methionine by stereospecific reductases MsrA and MsrB (FIG. 1). The observed methionine-sulfoxide proteome represents a steady-state condition in which oxidation, a chemical event, is balanced by reduction, an enzymatic process. The Msr system protects cells against oxidative damage by a reactive oxygen species (ROS)-scavenging mechanism in which methionine residues in proteins function as catalytic antioxidants, and the methionine sulfoxide reductase enzymes then repair the damage to the reversibly oxidized proteins. The enzymatic reversal of methionine oxidation with purified MsrA and MsrB enzymes has been demonstrated in vitro and detected by gel mobility shift, Western blotting with methionine sulfoxide-specific antibodies, and by MS analysis of intact proteins. While some Msr enzymes utilize DTT and other reducing agents in vitro without supplementary enzymes, other Msr enzymes rely on reducing enzymes, such as thioredoxin and thioredoxin reductase with NADPH and reducing buffer conditions (e.g. dithiothreitol, DTT) in the reaction to maintain Msr catalytic activity.

In some embodiments, the invention relates to the use of methionine sulfoxide reductase enzyme to reverse methionine oxidation in proteins and peptides prior to purification or analysis, such as liquid chromatography and/or mass spectrometry. In some embodiments, sample variability and complexity is reduced by the reversal of methionine oxidation and consolidation of multiple peptide species. In some embodiments, this enables easier interpretation of data, better sensitivity, and/or more accurate quantitation of proteins and peptides in samples, including biological samples.

In some embodiments, nucleic acids encoding the Msr proteins are provided. In some embodiments, Msr proteins are provided which have enzymatic activity in a reducing buffer. Further, in some embodiments, methods for treating protein and peptide samples prior to MS analysis are provided. The present disclosure demonstrates that Msr proteins effectively reverse methionine oxidation and improve protein or peptide sample quality and MS results.

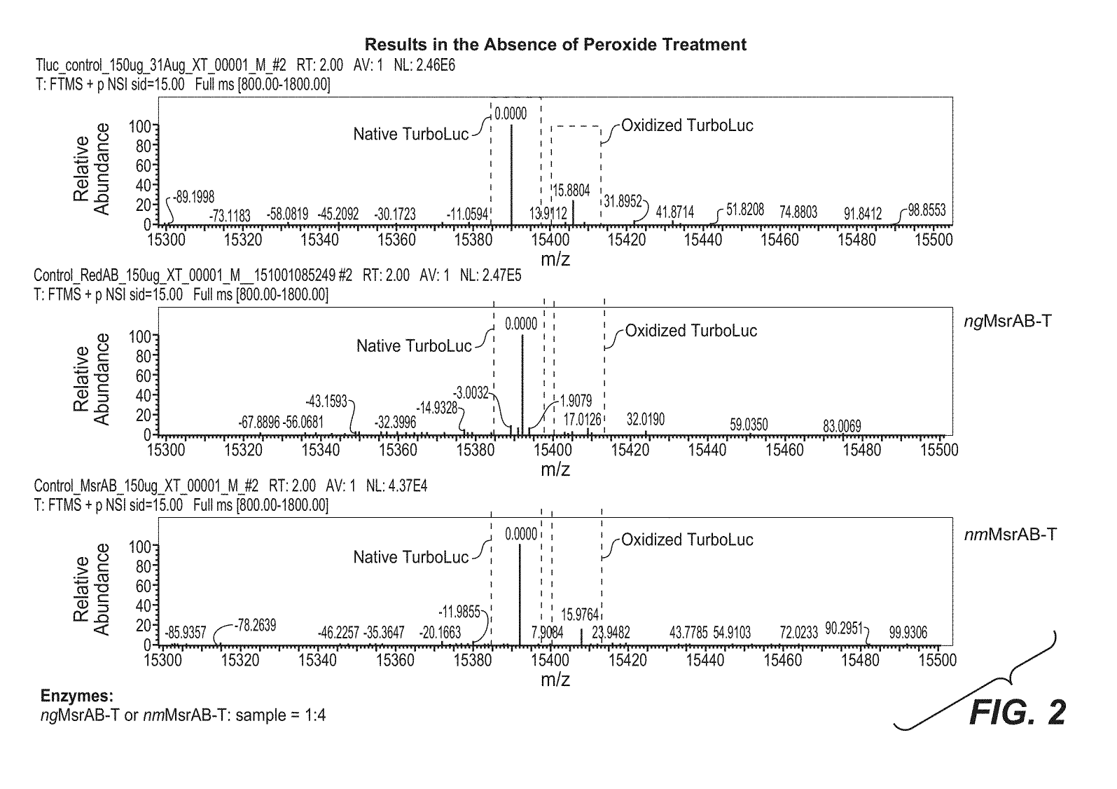

In some embodiments, the reversal of methionine oxidation reduces the number of proteoforms with different masses and the consolidation into fewer intact masses increases the intensity and signal to noise of each form. See, e.g., FIGS. 2 to 7. In some instances, each intact mass may be composed of multiple forms of a protein with the same total number of oxidized methionines but at different positions. Such proteoforms appear as one intact mass but can produce a complex combination of unique fragment ions during MS/MS. This increased complexity complicates the data analysis and reduces the sensitivity because of the dilution of signal across many species. In some embodiments, the reversal of methionine oxidation by the methods described herein reduces sample complexity, reduces the data analysis time, and improves the quality and confidence in the results. See FIG. 16.

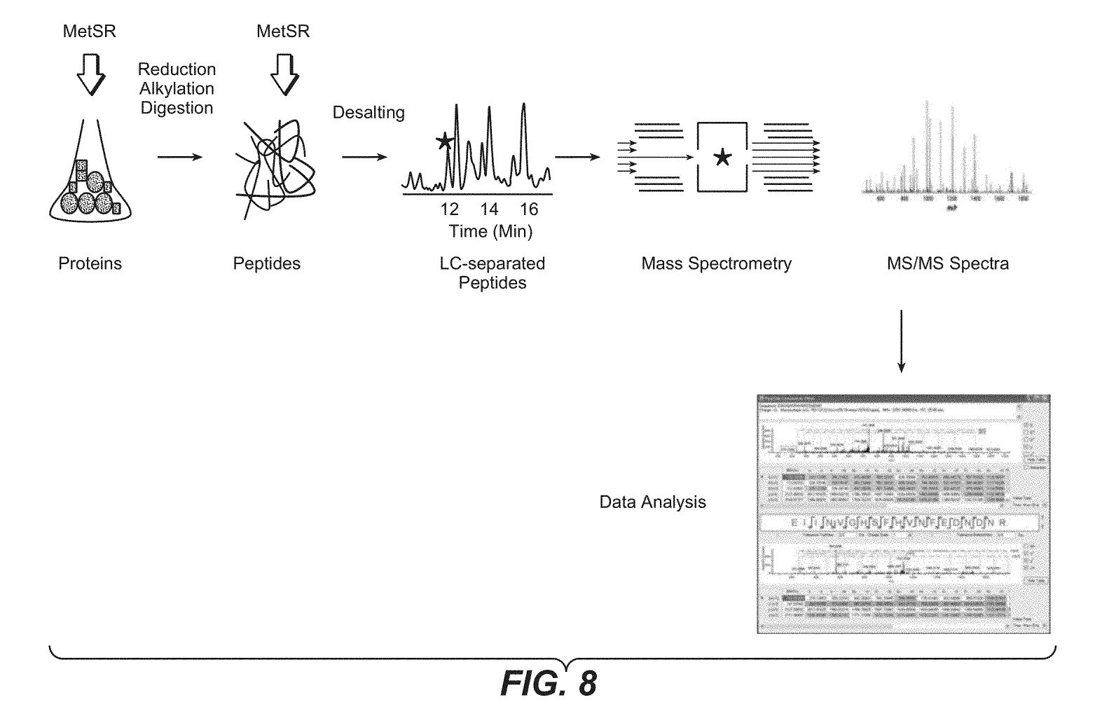

In some instances, it is found that 10-30% of methionines are oxidized in complex proteomic samples after reduction, alkylation, fragmentation (e.g., digestion), and desalting. Methionine oxidation may occur in the cell before lysis, during cell lysis, and/or during the subsequent sample preparation or analysis. In some embodiments, since methionine sulfoxide reductase can be functional during the reduction of protein disulfides in a mass spectrometry workflow, it may be possible to reduce cysteine disulfides and oxidized methionine simultaneously. See FIG. 8. In some embodiments, after fragmentation (e.g., digestion), peptide samples are analyzed by mass spectrometry (MS), and the resulting spectra are compared with theoretical spectra from known proteins to determine the peptides and proteins in a sample. To avoid missing peptides, in some instances, MS database searches permit methionine oxidation as a variable modification, but such inclusion may double the database search time. Thus, in some embodiments, the methods provided herein can simplify the MS database searches and reduce the database search time. That is, in some embodiments, by reversing methionine oxidation, sample complexity is reduced, improving the database search scores, e.g., by reducing the degrees of freedom and number of false positive hits, and shortening the database search time by eliminating the need to search for methionine oxidation as a variable modification.

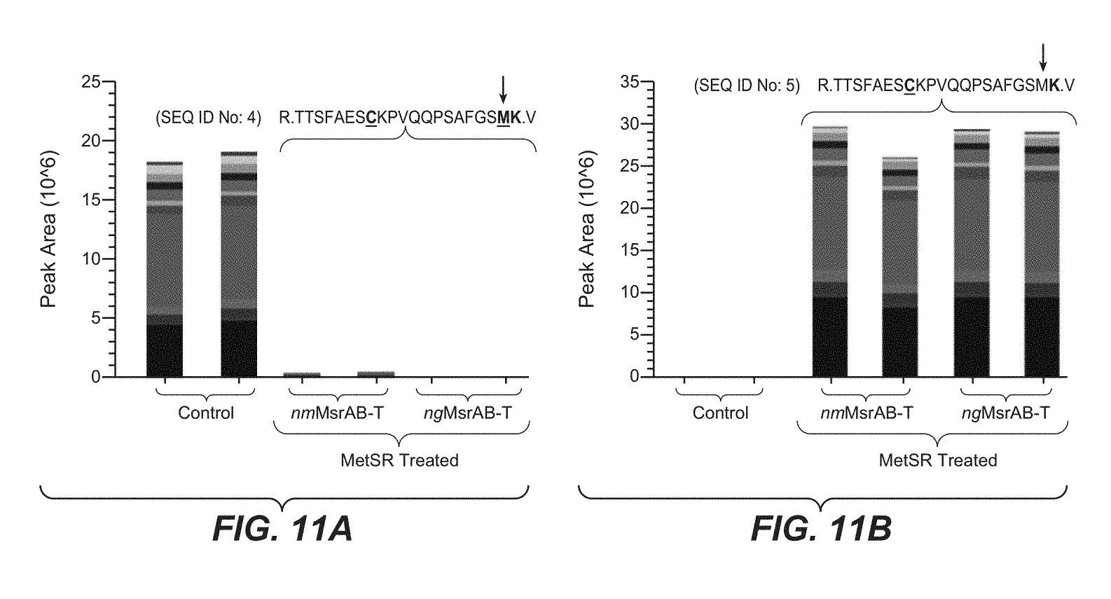

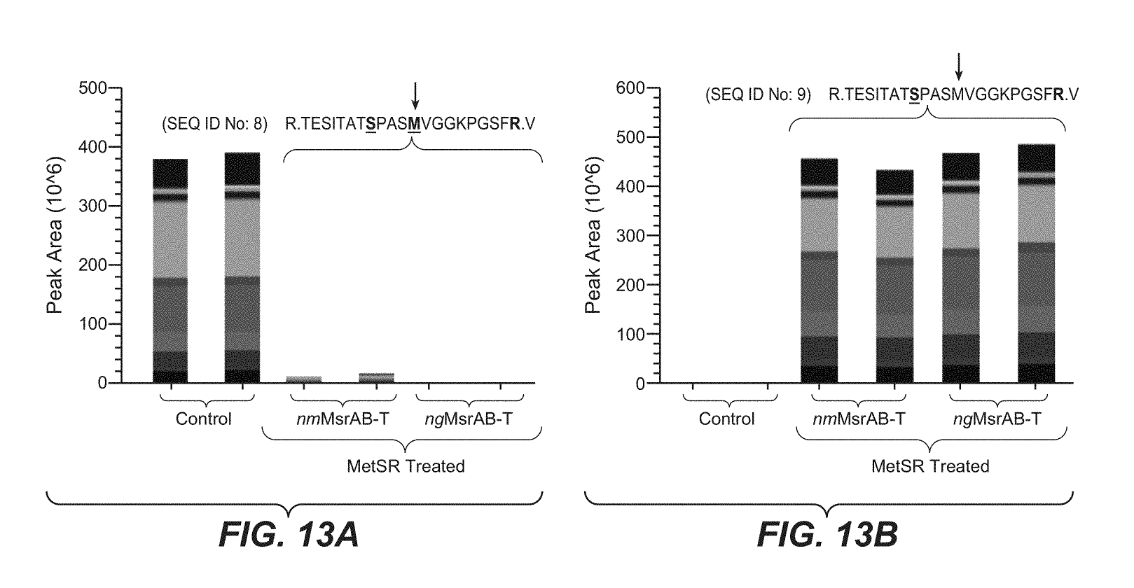

Targeted quantitation of proteins with mass spectrometry is typically performed by quantifying specific unique peptides of the protein. In some embodiments, known amounts of isotope-labeled (e.g., heavy isotope-labeled) versions of these targeted peptides can be used as internal standards for absolute quantitation. In some instances, when choosing peptides for targeted MS assays, peptides containing methionine are avoided because of the potential for oxidation and the resulting variability in quantitative measurements. The avoidance of methionine-containing peptides limits the choice of peptides that can be used to quantify proteins, and may prevent the quantitation of specific peptides of interest, such as when methionine-containing peptides also contain important signaling or regulatory modifications, such as phosphorylation, methylation, acetylation, or ubiquitinylation. In some embodiments, treatment using methionine sulfoxide reductases according to the methods described herein can reverse methionine oxidation and permit the targeted quantification of methionine-containing peptides. See, e.g., FIGS. 11 to 13. In some embodiments, methionine oxidation can be removed without altering other modifications, so methionine sulfoxide reductase treatment permits methionine-containing peptides to be monitored with targeted MS assays that may be otherwise difficult or impossible to measure. See, e.g., FIG. 13.

Nonlimiting exemplary Msr enzymes are described herein, and include MsrABs comprising the sequences of SEQ ID NOs: 10-34, and MsrABs that are at least 70%, at least 80%, at least 90%, at least 91%, at least 92%, at least 93%, at least 94%, at least 95%, at least 96%, at least 97%, at least 98%, at least 99%, or 100% identical to a sequence selected from SEQ ID NOs: 10-34.

In some embodiments, the Msr enzyme is derived from a bacterial Msr enzyme. In some embodiments, the Msr enzyme is derived from a bacteria selected from Neisseria, Lautropia, Cardiobacterium, Gammaproteobacteria, Pelistega, Marinospirillum, Basilea, Oligella, Alcagenaceae, Psychrobacter, Brackiella, Taylorella, Moraxella, Enhydrobacter, Fusobacterium, Helcococcus, Paenibacillus, and Eremococcus. One skilled in the art can identify Msr enzymes from various bacterial sources. In some embodiments, the Msr enzyme is derived from a bacterial MsrAB enzyme, i.e., a bacterial enzyme comprising a methionine sulfoxide reductase A domain and a methionine sulfoxide reductase B domain. The bacterial Msr enzyme may optionally comprise a thioredoxin domain. In some embodiments, the Msr enzyme is derived from a Neisseria bacteria. In some embodiments, the Msr enzyme is derived from Neisseria gonorrhoeae, Neisseria meningitides, Neisseria lactamica, Neisseria polysaccharea, Neisseria flavescens, Neisseria sicca, Neisseria macacae, or Neisseria mucosa.

In this application, “ng” denotes an MsrAB enzyme from Neisseria gonorrhoeae (e.g., ngMsrAB or ngMsrAB-T). In this application, “nm” denotes an MsrAB enzyme from Neisseria meningitides (e.g., nmMsrAB or nmMsrAB-T).

In some embodiments, the Msr enzyme comprises an amino acid sequence that is at least 70%, at least 80%, at least 90%, at least 91%, at least 92%, at least 93%, at least 94%, at least 95%, at least 96%, at least 97%, at least 98%, at least 99%, or 100% identical to a sequence selected from SEQ ID NOs: 10-34.

In some embodiments, the Msr enzyme is from a nonbacterial organism. In some embodiments, the Msr enzyme is derived from an Msr enzyme of an organism selected from Methanobrevibacter, Methanomassiliicoccales, Methanocorpusculum, Thermoplasmatales, Methanometylophilus, Methanoculleus, Methanocella, and the like. In some such embodiments, the Msr enzyme is an MsrAB enzyme. One skilled in the art can identify suitable MsrAB enzymes for use in the present methods. In some embodiments, the Msr enzyme from a non-Neisseriaceae bacteria or nonbacterial organism comprises an amino acid sequence that is at least 70%, at least 80%, or at least 90%, at least 91%, at least 92%, at least 93%, at least 94%, at least 95%, at least 96%, at least 97%, at least 98%, at least 99%, or 100% identical to a sequence selected from SEQ ID Nos: 10-34.

In some embodiments, the Msr enzyme is an MsrA enzyme or an MsrB enzyme. One skilled in the art can identify suitable MsrA and/or MsrB enzymes for use in the methods described herein. In some embodiments, the MSR-A comprises a peptide-methionine (S)-S-oxide reductase. In some embodiments, an MsrA is derived from an MsrA enzyme of an organism selected from Haloarcula, Halococcus, Haloferax, Natronococcus, Natronomonas, and Natrinema. Nonlimiting exemplary such MsrA enzymes can be found in various protein databases and include, for example, MsrA enzymes under accession numbers WP_049944603.1, WP_005043086.1, WP_058572480.1, WP_015322392.1, WP_015408133.1, and WP_006431385.1. In some embodiments, an MsrA is at least 70%, at least 80%, or at least 90%, at least 91%, at least 92%, at least 93%, at least 94%, at least 95%, at least 96%, at least 97%, at least 98%, at least 99%, or 100% identical to an MsrA enzyme under accession number WP_049944603.1, WP_005043086.1, WP_058572480.1, WP_015322392.1, WP_015408133.1, or WP_006431385.1. In some embodiments, the Msr-B comprises a peptide-methionine (R)-S-oxide reductase. In some embodiments, an MsrB is derived from an MsrB enzyme of an organism selected from Haloarcula, Halococcus, Haloferax, Natronococcus, Natronomonas, Natrinema, and Candidatus Halobonum. Nonlimiting exemplary such MsrB enzymes can be found in various protein databases and include, for example, MsrB enzymes under accession numbers WP_004963222.1, WP_049996544.1, WP_007275637.1, WP_008423757.1, WP_015408129.1, WP_007109050.1, and WP_023395429.1. In some embodiments, an MsrB is at least 70%, at least 80%, or at least 90%, at least 91%, at least 92%, at least 93%, at least 94%, at least 95%, at least 96%, at least 97%, at least 98%, at least 99%, or 100% identical to an MsrA enzyme under accession number WP_004963222.1, WP_049996544.1, WP_007275637.1, WP_008423757.1, WP_015408129.1, WP_007109050.1, or WP_023395429.1. In some embodiments, MsrA and MsrB are derived from MsrA and MsrB enzymes of the same organism. In some embodiments, MsrA and MsrB are derived from MsrA and MsrB enzymes of the different organisms. In some embodiments, MsrA and MsrB are derived from MsrA and MsrB enzymes of organisms of the same genus, but different species.

As used herein, an Msr enzyme that is “derived from” an Msr enzyme of a particular organism or of a particular sequence may be modified, such as by truncation or addition of amino acids (such as addition of a tag sequence and/or protease sequence for removal of the tag) relative to the parental Msr enzyme, but retains at least MsrA or MsrB activity. In some embodiments, the Msr enzyme derived from an Msr enzyme of a particular organism or of a particular sequence retains at least 50% of the MsrA or MsrB activity (but not necessarily both) of the parental enzyme.

In some embodiments, a method comprises contacting a polypeptide sample with a mixture of different Msr's. In some embodiments, a single Msr is used. In some embodiments, when a mixture of Msrs is used, the mixture comprises at least one MsrA and at least one MsrB. In some embodiments, a method comprises contacting a polypeptide sample with an MsrAB, with or without additional Msrs. In some embodiments, an MsrAB may be used in conjunction with an MsrA and/or an MsrB.

In some embodiments, an Msr:protein ratio (w/w) of 1:100 to 1:2 in used. In some embodiments, an Msr:protein ratio (w/w) of 1:2, 1:4, 1:10, 1:20, 1:25, 1:50, 1:66, 1:75 or 1:100 is used. In some embodiments, the Msr used is at a concentration of about 100 ng/ml-1 mg/ml, or about 100 ng/ml-500 μg/ml, or about 100 ng/ml-100 μg/ml, or about 1 μg/ml-1 mg/ml, or about 1 μg/ml-500 μg/ml, or about 1 μg/ml-100 μg/ml, or about 10 μg/mg-1 mg/ml, or about 10 μg/mg-500 μg/ml, or about 10 μg/mg-100 μg/ml. In some embodiments, the method comprises contacting a polypeptide sample with at least one Msr under conditions suitable for reduction of methionine sulfoxides for 10 minutes to 48 hours, or 30 minutes to 48 ours, or 30 minutes to 24 hours, or 30 minutes to 16 hours, or 1 hour to 48 hours, or 1 hour to 24 hours, or 1 hour to 16 hours, or 1 to 8 hours, or 1 to 6 hours, or 1 to 4 hours. In some embodiments, the Msr reaction is incubated at a temperature between 20° C. and 45° C., or between 20° C. and 40° C., or between 22° C. and 40° C., or between 25° C. and 37° C. In some embodiments, the Msr reaction is incubated at 37° C. or 30° C.

In some embodiments, contacting the Msr with the polypeptide sample occurs under reducing conditions. In some embodiments, contacting of the Msr with the protein sample occurs in the presence of dithiothreitol (DTI) or dithioerythritol (DTE).

In some embodiments, the Msr reaction is terminated. In some embodiments the Msr is removed following reduction of oxidized methionines in the protein sample. In some embodiments the Msr is removed by spinning or pelleting of the sample. In some such embodiments, the Msr enzyme is bound to a solid support, such as a resin or bead. The step to terminate the Msr reaction may occur before, after, or concurrently with a treatment to fragment (e.g., digest) the protein sample.

Described herein are uses of methionine sulfoxide reductase (Msr) enzymes in the preparation of protein samples before purification or analysis, such as for liquid chromatography and/or mass spectrometry analysis. The reduction of oxidized methionine residues using Msr enzymes is shown to improve liquid chromatography and/or mass spectrometry data and simplify analysis.

Mass spectrometry (MS) is a primary technique for analysis of proteins on the basis of their mass-to-charge ratio (m/z). MS techniques generally include ionization of compounds and optional fragmentation of the resulting ions, as well as detection and analysis of the m/z of the ions and/or fragment ions followed by calculation of corresponding ionic masses. A “mass spectrometer” generally includes an ionizer and an ion detector. “Mass spectrometry,” “mass spec,” “mass spectroscopy,” and “MS” are used interchangeably throughout.

The methods disclosed herein may be applied to any type of MS analysis. The invention is not limited by the specific equipment or analysis used. The use of any equipment with the intent of analyzing the m/z of a sample would be included in the definition of mass spectrometry. Non-limiting examples of MS analysis and/or equipment that may be used include electrospray ionization, ion mobility, time-of-flight, tandem, ion trap, and Orbitrap. The invention is neither limited by the type of ionizer or detector used in the MS analysis nor by the specific configuration of the MS. The invention is not limited to use with the specific equipment and analysis described in the Examples.

In some embodiments, the invention comprises use of an Msr enzyme for preparing a protein sample for top-down MS analysis, wherein the protein sample is contacted with an Msr enzyme prior to MS analysis. In some embodiments, the protein sample is intact (e.g., not fragmented) when contacted with an Msr enzyme. In some embodiments, the protein sample is not intact (e.g., fragmented) prior to Msr enzyme contact. In some embodiments, the invention comprises use of an Msr enzyme for preparing a protein sample for MS analysis, wherein the MS analysis comprises the step of disassociating intact protein or protein complexes. In some embodiments, the use comprises internal fragmentation of the proteins of the sample. The internal fragmentation step may be accomplished by way of Collision Induced Dissociation (CID), Electron Capture Dissociation (ECD), Electron Transfer Dissociation (ETD), or Surface Induced Dissociation (SID), for example. In some embodiments, the disassociating step is prior to Msr enzyme contact, whereas in some embodiments the disassociating step is after Msr enzyme contact.

In “top-down” mass spectrometry analysis, intact proteins and/or protein complexes are subjected to fragmentation inside the mass spectrometer. In some instances, top-down analysis preserves the post-translationally modified forms of proteins. Further, in some instances, top-down analysis may provide close to 100% sequence coverage and may facilitate the study of coordinated regulation of multiple modification sites within a single protein. In some embodiments, top-down analysis has the ability to detect protein degradation products, sequence variants, and combination of post-translational modifications and their locations within the intact protein. Methionine oxidation presents a problem for top-down analysis because it increases sample complexity data analysis dramatically. For example, in some instances, each oxidized methionine can split the MS signal (e.g., into non-oxidized and oxidized peaks), a problem that is magnified by the number of methionines in a protein.

In “bottom-up” mass spectrometry analysis, proteins in a sample are fragmented, for example, by enzymatic digestion using enzymes such as trypsin, and then identified, in some embodiments, using high performance liquid chromatography combined with mass spectrometry. In some embodiments, proteins are denatured, reduced to remove disulfide bonds, and then free cysteines are alkylated to prevent formation of new disulfide bonds. In some embodiments, the proteins are then fragmented. In some embodiments, the resulting peptides are then separated by liquid chromatography. Mass spectrometry may then be used to identify the peptides, e.g., by matching the fragmentation pattern to theoretical tandem mass spectrometry databases.

Use of an Msr enzyme for preparing a protein sample may be combined with any other steps taken to prepare samples for MS analysis.

In some embodiments, proteins within the sample are separated from other components of samples. In some embodiments, the proteins in the sample are not fragmented. In some embodiments, proteins in the sample are subjected to liquid chromatography before or after reduction of methionine sulfoxides according to the present methods. In some embodiments, proteins in the sample are subjected to capillary electrophoresis before or after reduction of methionine sulfoxides according to the present methods.

In some embodiments, liquid chromatography (LC) is used for physical separation of protein samples. When LC is performed at relatively high pressure, it may be termed high performance liquid chromatography (HPLC). Nonlimiting exemplary LC includes reversed phase LC (RP-LC) and normal phase LC (NP-LC). In some embodiments, capillary electrophoresis (CE) is used for physical separation of protein samples.

In some embodiments, LC may be used in conjunction with MS. In some such embodiments, an LC system may be linked to an MS (i.e., LC-MS or HPLC-MS), see Thompson M, AMC Technical Brief #34, 2008. In some embodiments, capillary electrophoresis (CE) may be used in conjunction with MS. In some embodiments, the LC-MS ionization technique is electrospray ionization (ESI), atmospheric pressure chemical ionization (APCI), or atmospheric pressure photoionization (APPI), see Basics of LCIMS, Agilent Technologies, 2001. Any type of mass analyzer may be used for LC-MS, including quadrupole, time-of-flight, ion trap, or Fourier transform-ion cyclotron resonance (FT-ICR or FT-MS). In some embodiments, LC-MS includes internal fragmentation during the MS analysis.

In some embodiments, the protein sample comprises fragmented protein. In some embodiments, the fragmented protein sample includes proteins or peptides of a size that can be analyzed by the selected method. In some embodiments, a fragmented protein sample comprises peptide of 5 to 100 amino acids in length. In some embodiments, the protein sample comprises predominantly peptides of between 2-100, 5-100, 5-90, 5-80, 5-70, 5-60, 5-50, 5-40, 5-30, 10-90, 10-80, 10-70, 10-60, 10-50, 10-40, 10-30, or 10-20 amino acids in length. In some embodiments, the protein fragments comprises predominantly peptides of 5 to 50 amino acids in length.

In some embodiments, the protein sample comprises protein fragments. In some such embodiments, the protein fragments are generated by an enzyme. In some embodiments, the protein sample comprises fragmented protein (e.g., protease-digested protein fragments).

In some embodiments, a method provided herein comprises use of an Msr for preparing a protein sample for bottom-up MS analysis. In some embodiments, the protein sample contacted with the Msr is fragmented before or after treatment with the Msr.

In some embodiments, a method provided herein comprises use of an Msr for preparing a protein sample for top-down MS analysis. In some embodiments, the protein is contacted with the Msr enzyme prior to fragmentation.

In some embodiments, protein samples are denatured or solubilized before fragmentation.

In some embodiments, the fragmentation protocol uses chemical cleavage. In some embodiments, the chemical cleavage uses CNBr. In some embodiments, the fragmentation protocol is done using an enzyme. In some embodiments, the fragmentation protocol uses MS-grade commercially available proteases. Examples of proteases that may be used to digest samples include trypsin, endoproteinase GluC, endoproteinase ArgC, pepsin, chymotrypsin, LysN protease, LysC protease, GluC protease, AspN protease, proteinase K, and thermolysin. In some embodiments, a mixture of different proteases are used and the individual results are combined together after the digestion and analysis. In some embodiments, the digestion is incomplete in order to see larger, overlapping peptides. In some embodiments, the antibody digestion is performed with IdeS, IdeZ, pepsin, or papain to generate large antibody domains for “middle-down” protein characterization. In some embodiments, the fragmentation protocol uses trypsin that is modified. In some embodiments, a protein:protease ratio (w/w) of 10:1, 20:1, 25:1, 50:1, 66:1, or 100:1 may be used. In some embodiments, the trypsin used is at a concentration of about 100 ng/ml-1 mg/ml, or about 100 ng/ml-500 μg/ml, or about 100 ng/ml-100 μg/ml, or about 1 μg/ml-1 mg/ml, or about 1 μg/ml-500 μg/ml, or about 1 μg/ml-100 μg/ml, or about 10 μg/mg-1 mg/ml, or about 10 μg/mg-500 μg/ml, or about 10 μg/mg-100 μg/ml. In some embodiments, the digestion step is for 10 minutes to 48 hours, or 30 minutes to 48 ours, or 30 minutes to 24 hours, or 30 minutes to 16 hours, or 1 hour to 48 hours, or 1 hour to 24 hours, or 1 hour to 16 hours, or 1 to 8 hours, or 1 to 6 hours, or 1 to 4 hours. In some embodiments, the digestion step is incubated at a temperature between 20° C. and 45° C., or between 20° C. and 40° C., or between 22° C. and 40° C., or between 25° C. and 37° C. In some embodiments, the digestion step is incubated at 37° C. or 30° C. In some embodiments, a step is included to end the digestion step. The step to end the digestion protocol may be addition of a stop solution or a step of spinning or pelleting of a sample. The step to end the digestion step may occur before, after, or concurrently with treatment with the Msr. In some embodiments, the digestion is followed by guanidation.

In some embodiments, the fragmentation protocol includes use of protein gels. In some embodiments, the fragmentation protocol comprises in-gel digestion. An exemplary commercially available kit for performing in-gel digestion is the In-Gel Tryptic Digestion Kit (Thermo Fisher Cat#89871).

In some embodiments, the fragmentation protocol is carried out in solution. An exemplary commercially available kit for performing in-solution digestion is the In-Solution Tryptic Digestion and Guanidiation Kit (Thermo Fisher Cat#89895).

In some embodiments, the fragmentation protocol uses beads. In some embodiments, the fragmentation protocol comprises on-bead digestion. In some embodiments, agarose beads or Protein G beads are used. In some embodiments, magnetic beads are used.

In some embodiments, protein samples are separated using liquid chromatography before MS analysis. In some embodiments, fragmented samples are separated using liquid chromatography before MS analysis.

In some embodiments, kits are provided, comprising at least one methionine sulfoxide reductase (Msr) enzyme. In some embodiments, the at least one methionine sulfoxide reductase enzyme is capable of reducing methionine-S-sulfoxide, or is capable of reducing methionine-R-sulfoxide, or is capable of reducing both methionine-S-sulfoxide and methionine-R-sulfoxide. In some embodiments, the kit comprises at least one methionine sulfoxide reductase enzyme that is capable of reducing methionine-S-sulfoxide and at least one methionine sulfoxide reductase enzyme that is capable of reducing methionine-R-sulfoxide. In some such embodiments, the kit comprises an MsrA enzyme and an MsrB enzyme. In some embodiments, the kit comprises at least one methionine sulfoxide reductase enzyme that is capable of reducing both methionine-S-sulfoxide and methionine-R-sulfoxide, such as an MsrAB enzyme.

Nonlimiting exemplary Msr enzymes that may be included in kits are described herein, and include, for example, MsrAB enzymes derived from a methionine sulfoxide reductase from an organism selected from Neisseria, Lautropia, Cardiobacterium, Gammaproteobacteria, Pelistega, Marinospirillum, Basilea, Oligella, Alcagenaceae, Psychrobacter, Brackiella, Taylorella, Moraxella, Enhydrobacter, Fusobacterium, Helcococcus, Paenibacillus, Eremococcus, Methanobrevibacter, Methanomassiliicoccales, Methanocorpusculum, Thermoplasmatales, Methanometylophilus, Methanoculleus, and Methanocella. In some embodiments, the MsrAB enzyme is derived from a bacterial enzyme. In some embodiments, the methionine sulfoxide reductase enzyme is derived from a methionine sulfoxide reductase enzyme of Neisseria gonorrhoeae, Neisseria meningitides, Neisseria lactamica, Neisseria polysaccharea, Neisseria flavescens, Neisseria sicca, Neisseria macacae, or Neisseria mucosa. In some embodiments, the methionine sulfoxide reductase enzyme comprises an amino acid sequence that is at least 70%, at least 80%, at least 90%, at least 91%, at least 92%, at least 93%, at least 94%, at least 95%, at least 96%, at least 97%, at least 98%, at least 99%, or 100% identical to a sequence selected from SEQ ID NOs: 10 to 34. In some embodiments, the one or more Msr enzymes in a kit may be bound to a solid support, such as a resin or bead.

Kits may further comprises one or more additional reagents for preparing a polypeptide sample for liquid chromatography. Kits may further comprises one or more additional reagents for preparing a polypeptide sample for mass spectrometry analysis. In some embodiments, a kit comprises at least one reagent for fragmenting a polypeptide sample for mass spectrometry analysis. Nonlimiting exemplary reagents for fragmenting a polypeptide sample include trypsin, chymotrypsin, AspN, GluC, LysC, LysN, ArgC, proteinase K, thermolysin, and CNBr.

### EXAMPLES

The following examples are provided to illustrate certain disclosed embodiments and are not to be construed as limiting the scope of this disclosure in any way.

### Example 1. Intact Protein Analysis and Top-Down Analysis of Methionine Oxidation of TurboLuc by Hydrogen Peroxide

Methionine oxidation causes signal splitting of mass spectrometry (MS) of intact proteins, thus leading to increases in data complexities. Means of reducing methionine oxidation may thus improve MS data. Therefore, a model system was developed to study methionine oxidation and reduction that uses H₂O₂, which has been described as a means of monitoring methionine oxidation of methionine-rich proteins (see Le D T et al., Biochemistry 47(25):6685-6694 (2008) and Liang et al., BMC Biochemistry 13:21 (2012)).

As shown in FIG. 1, oxidized methionines (i.e., methionine-S-sulfoxide and methionine-R-sulfoxide) can be reduced by methionine sulfoxide reductases (Msrs), such as MetSR-A and MetSR-B. The ability of Msr treatment to improve MS data by reduction of methionine oxidation was investigated using a variety of MS procedures.

Turboluciferase (TurboLuc, SEQ ID No: 3) was selected for optimization of methionine oxidation and reduction reactions due to its compatibility with mass spectrometry. Initial experiments showed that treatment of 5 μg/ml of TurboLuc with 1 mM, 10 mM, or 100 mM hydrogen peroxide reduced the activity of TurboLuc by greater than 1000-fold using the TurboLuc flash assay (data not shown), indicating that TurboLuc was susceptible to oxidation.

For standardization of oxidation, a systematic ratio of moles of peroxide to moles of methionine must be maintained. Therefore, a concentration of 18 μM TurboLuc was used for all experiments. As there are three moles of methionine per mole of TurboLuc, the methionine concentration in experiments was 54 μM.

A variety of oxidation conditions were assessed by determining the number of moles of methionine in a sample and adding different ratios of moles of peroxide to each. After oxidation, each sample had DIT added to a final concentration of 5 mM while keeping the solution at a neutral pH and either ngMsrAB or nmMsrAB were added. Ratios of methionine:peroxide of 10:1, 1:1, 1:10, 1:25, 1:75, 1:100, and 1:500 were examined using the following conditions:

| | | | | ngMsrAB-T or |
|---|---|---|---|---|
| TLuc amount | Molar Ratio | Peroxide final | | nmMsrAB-T |
| (μg) | (Sample:Peroxide) | concentration | | added (μg) |
| 45 | 1:0 | 0 | | 11.25 |
| 45 | 10:1 | 5.4 | μM | 11.25 |
| 45 | 1:1 | 54 | μM | 11.25 |
| 45 | 1:10 | 540 | μM | 11.25 |
| 45 | 1:25 | 1.35 | mM | 11.25 |
| 45 | 1:50 | 2.7 | mM | 11.25 |
| 45 | 1:75 | 4.05 | mM | 11.25 |
| 45 | 1:100 | 5.4 | mM | 11.25 |
| 45 | 1:500 | 27 | mM | 11.25 |

The incubation with hydrogen peroxide was at 37° C. for 1 hour. The sample was spun down using a using a 3K MWCO buffer exchange protein concentrator (Fisher, Product #11345402) for 1 hour at 16,000×g. The sample was then buffer exchanged into a slightly basic solution of 50 mM Tris-HCl buffer and appropriate water to create a concentration of 0.5 μg/μL.

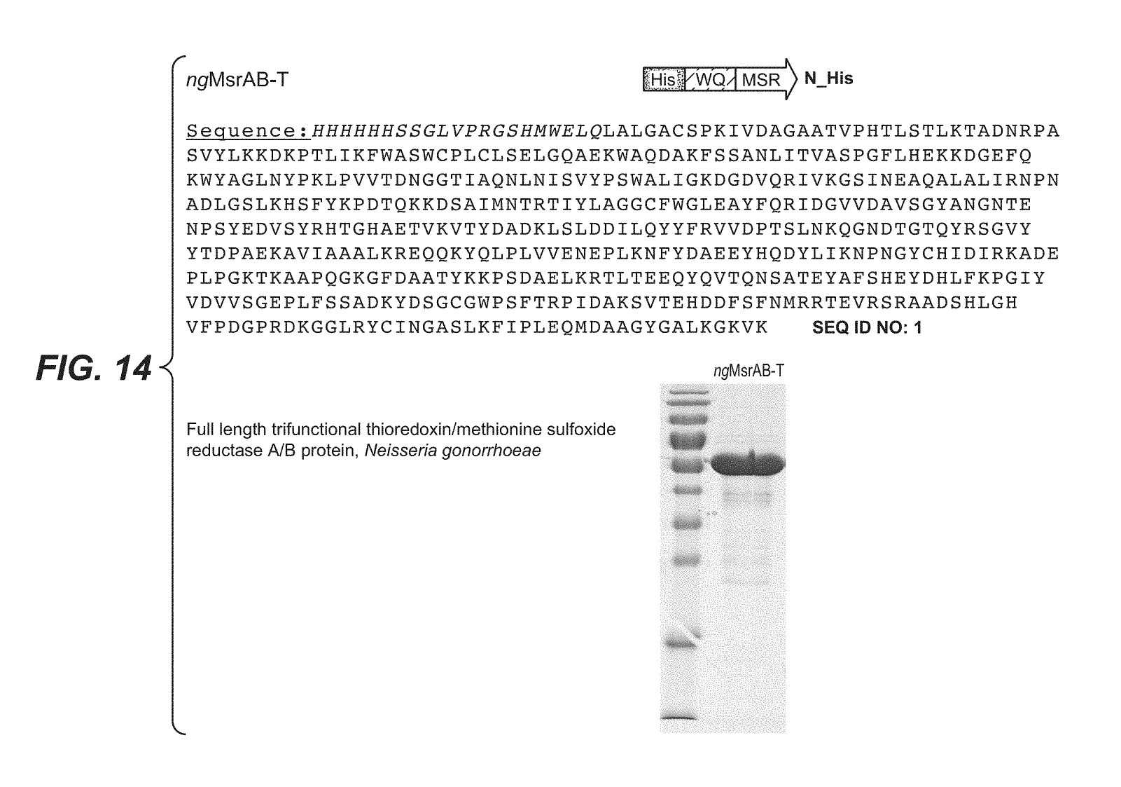

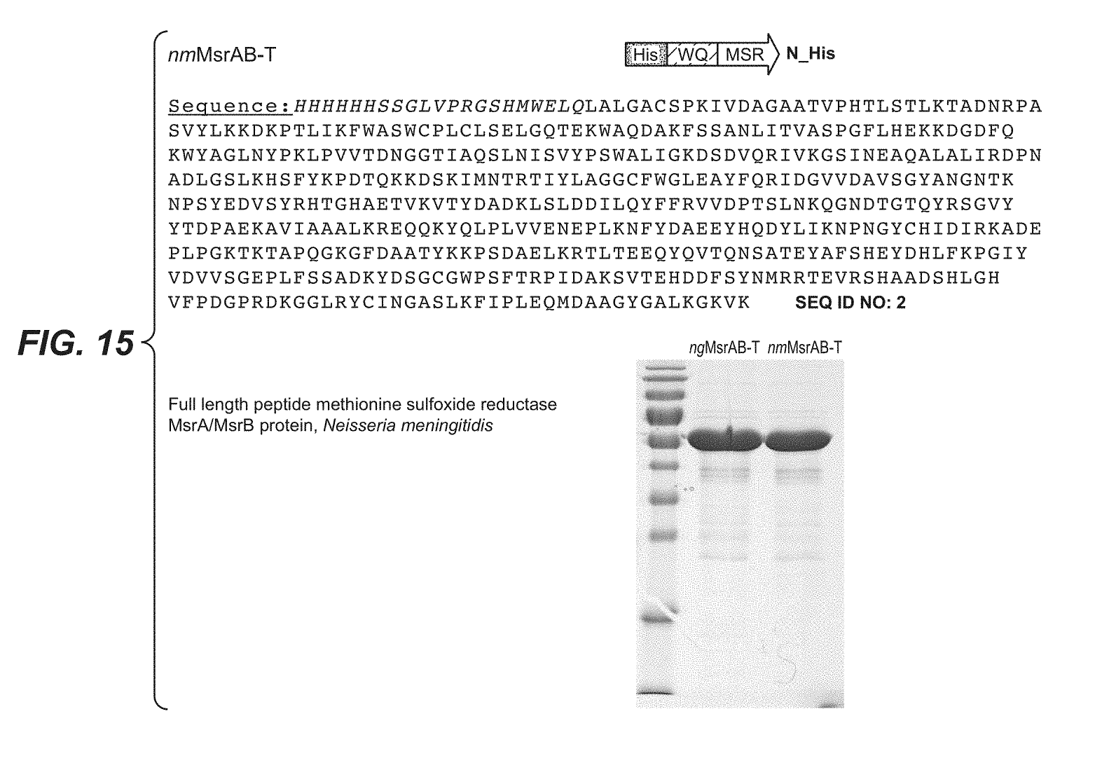

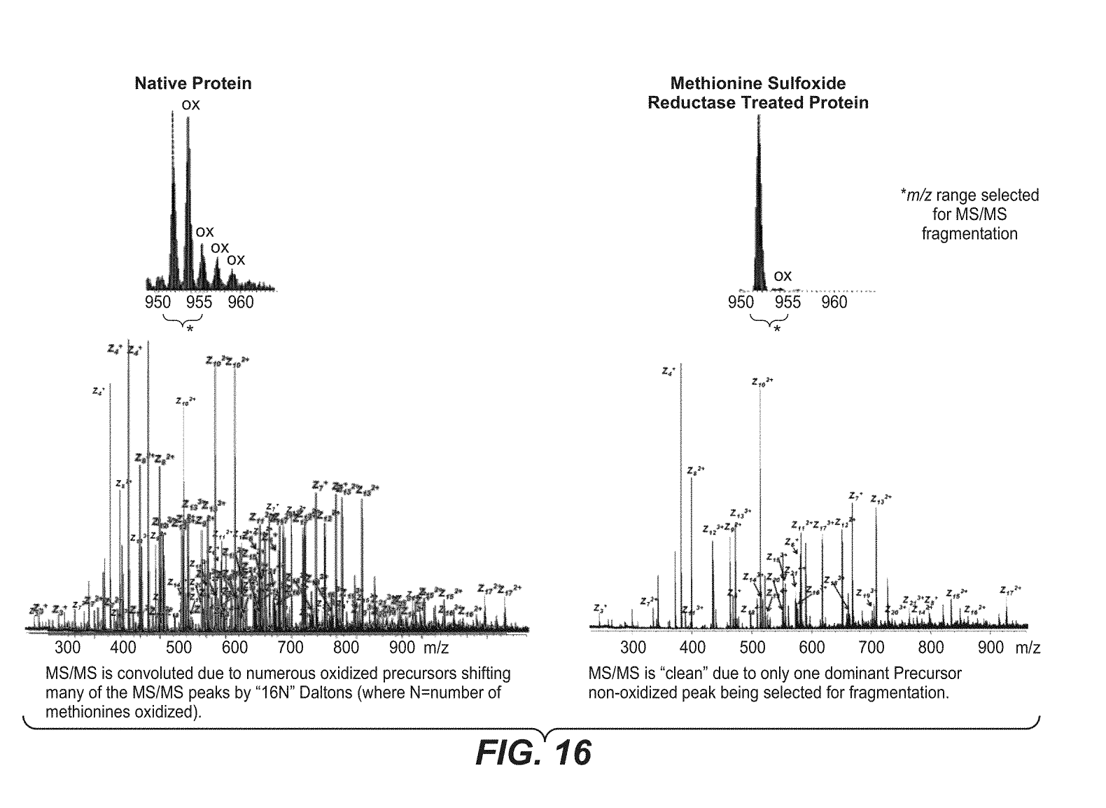

Reduction reactions were performed by reacting the oxidized sample with methionine sulfoxide reductase (Msr) enzymes, using either ngMsrAB (SEQ ID No: 1) or nmMsrAB (SEQ ID No: 2). As shown in FIG. 14, SEQ ID No: 1 is an N-terminal histidine tagged protein with a WQ domain fused to the full-length MsrAB from Neisseria gonorrhoeae, also known as “ngMsrAB”. As shown in FIG. 15, SEQ ID No: 2 is an N-terminal histidine tagged protein with a WQ domain fused to the full-length MsrAB from Neisseria meningitides, also known as “nmMsrAB”. The Msr enzyme fusion proteins of SEQ ID Nos: 1 and 2 were expressed, purified, and used for determining the effect of methionine reduction on MS profiles. Coomassie gels shown in FIGS. 14 and 15 indicate that a predominant protein of the expected molecular weight was obtained for SEQ ID No: 1 (“ngMsrAB”) and SEQ ID No: 2 (“nmMsrAB”).

To perform the reduction reactions, one of the two Msrs (either ngMsrAB [SEQ ID No: l] or nmMsrAB [SEQ ID No: 2]) was added to the oxidized TurboLuc solution at an enzyme:sample protein ratio of 1:4 and DTI of a final concentration of 5 mM. Both enzymes were used in separate experiments as shown below.

| | Starting | ngMsrAB-T | nmMsrAB-T | 5 mM DTT |
|---|---|---|---|---|
| Samples | Sample | volume added | volume added | (from 100 mM |
| (Methionine:Peroxide) | Volume (45 μg) | (11.25 μg total) | (11.25 μg total) | Stock) |
| Control | 90 μl | 7.5 μl | — | 4.9 μl |
| Control | 90 μl | — | 3.41 μl | 4.67 μl |
| 1:25 | 90 μl | 7.5 μl | — | 4.9 μl |
| 1:25 | 90 μl | — | 3.41 μl | 4.67 μl |
| 1:50 | 90 μl | 7.5 μl | — | 4.9 μl |
| 1:50 | 90 μl | — | 3.41 μl | 4.67 μl |
| 1:100 | 90 μl | 7.5 μl | — | 4.9 μl |
| 1:100 | 90 μl | — | 3.41 μl | 4.67 μl |

The samples plus Msr were incubated at 37° C. for 2 hours with gentle vortexing throughout. Samples were cleaned using 3K MWCO concentrators, and the volume was brought up to 200 μl with 0.1% formic acid.

Half of the cleaned-up intact protein sample was diluted to 40% acetonitrile (ACN), 60% 0.1% formic acid for direct injection into the Orbitrap XL mass spectrometer. Manual selection of peaks and MS/MS fragmentation were performed.

The other half of the cleaned-up intact protein sample was injected onto a ProSwift RP-4H 100 μm×25 cm monolithic column and separated by a gradient of water and acetonitrile in 0.1% formic acid. Automatic fragmentation was performed using data dependent analysis. Data analysis and visualization were done using ProSight Lite v1.2 Build 1.2.5595.15524 software that allows identification and characterization via intact protein analysis and top-down analysis.

FIG. 2 shows MS data in the absence of hydrogen peroxide for un-oxidized samples and for samples treated with ngMsrAB or nmMsrAB. Peaks corresponding to native TurboLuc and oxidized TurboLuc are indicated with boxes. These data indicate a relatively low level of baseline methionine oxidation of TurboLuc.

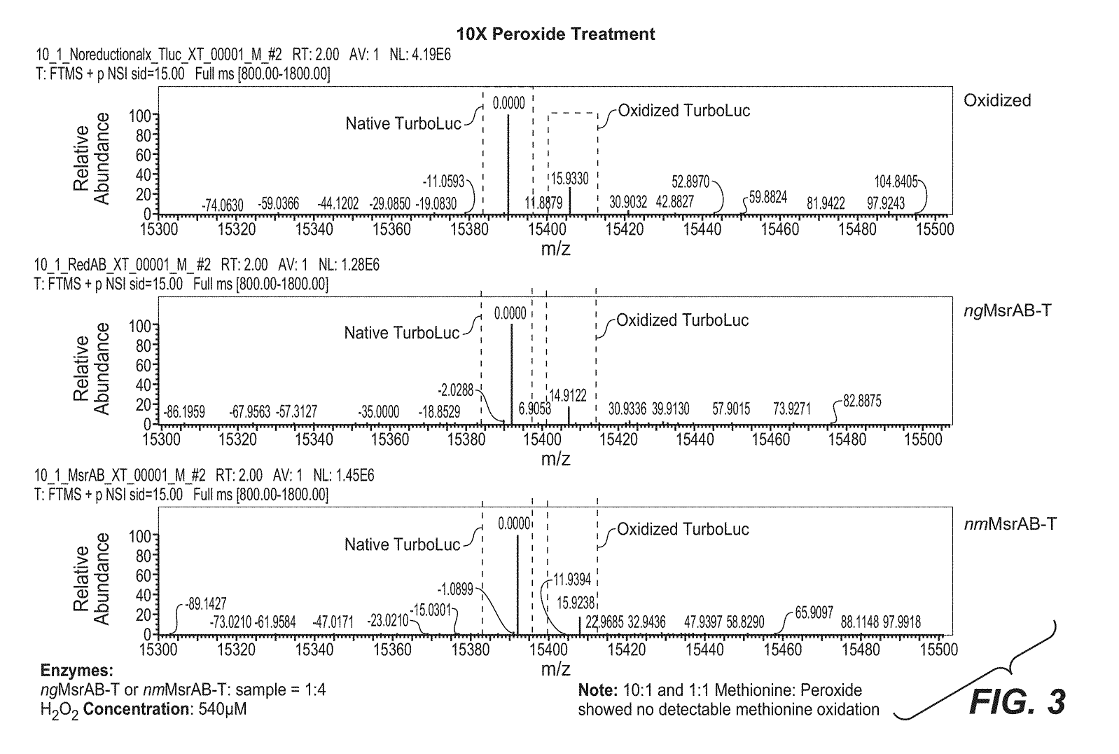

TurboLuc samples reacted with 10× peroxide treatment (10:1 peroxide:methionine, corresponding to 540 μM H₂O₂) are shown in FIG. 3. The 10:1 peroxide to methionine reaction conditions did not produce pronounced differences from those obtained in the absence of hydrogen peroxide (FIG. 2). In addition, the 10:1 and 1:1 methionine to peroxide reaction conditions showed no detectable methionine oxidation (data not shown).

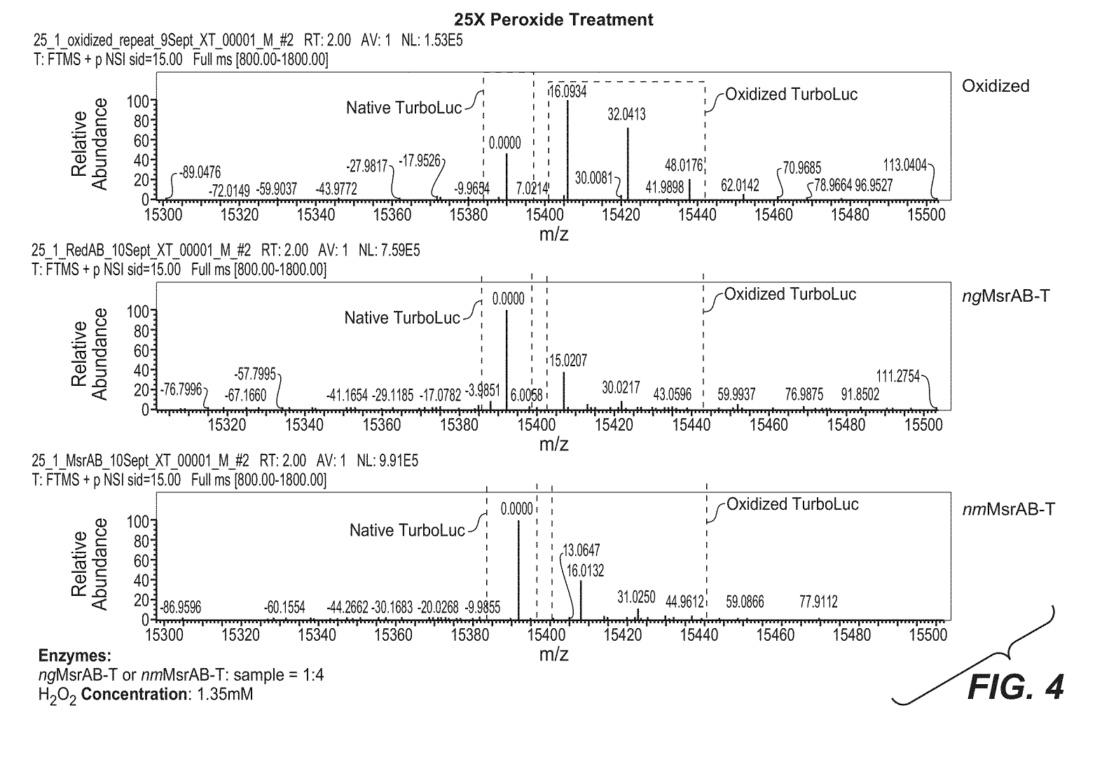

FIG. 4 indicates that 25× peroxide treatment (25:1 peroxide:methionine corresponding to 1.35 mM H₂O₂) increased methionine oxidation of TurboLac. This is apparent in new peaks in spectrum region corresponding to oxidized TurboLac as well as larger relative abundance of the peaks corresponding to oxidized TurboLuc. Treatment with ngMsrAB or nmMsrAB (at a ratio of 1:4 of enzyme to sample) counteracted the effect of 25× peroxide treatment, and the MS data obtained with these samples were similar to those shown in FIG. 2 for un-oxidized sample not reacted with hydrogen peroxide.

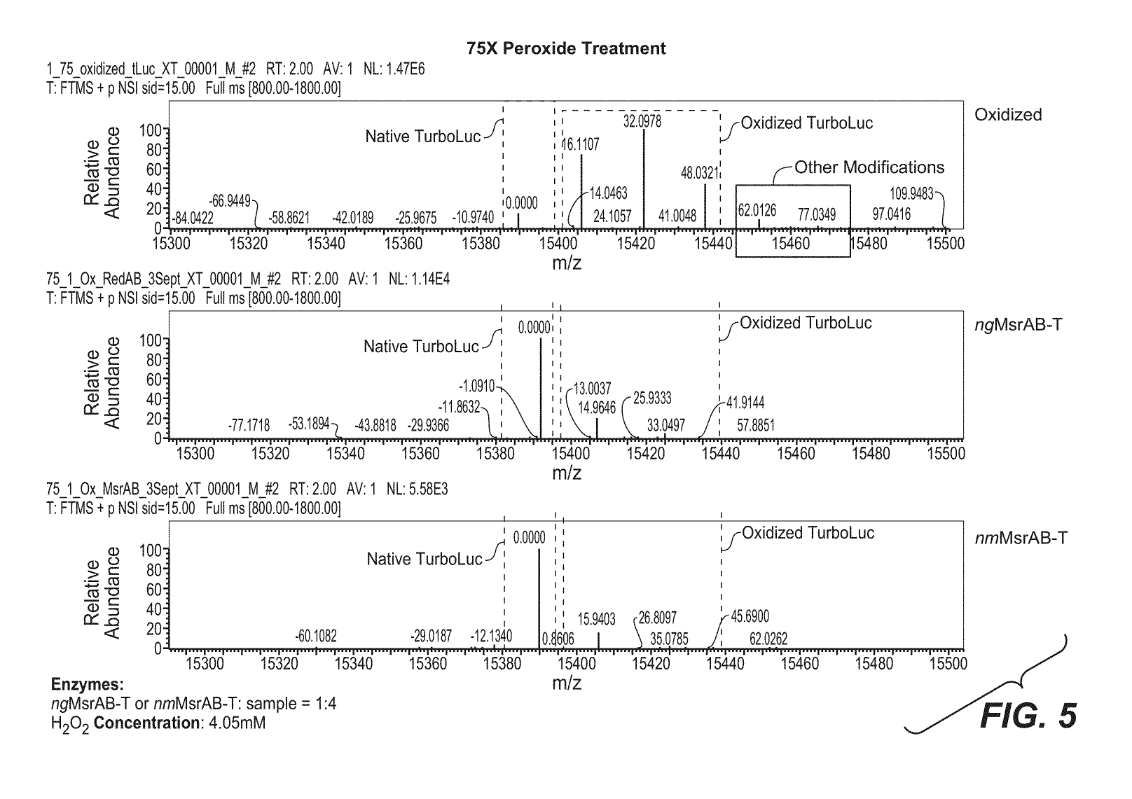

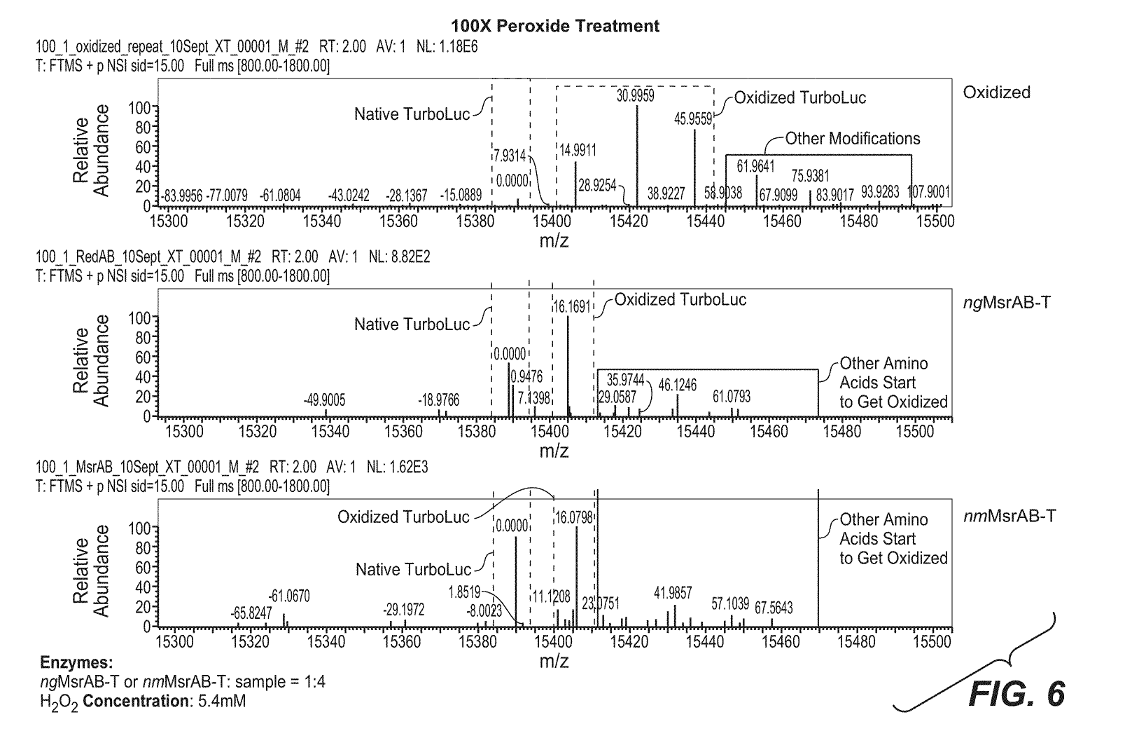

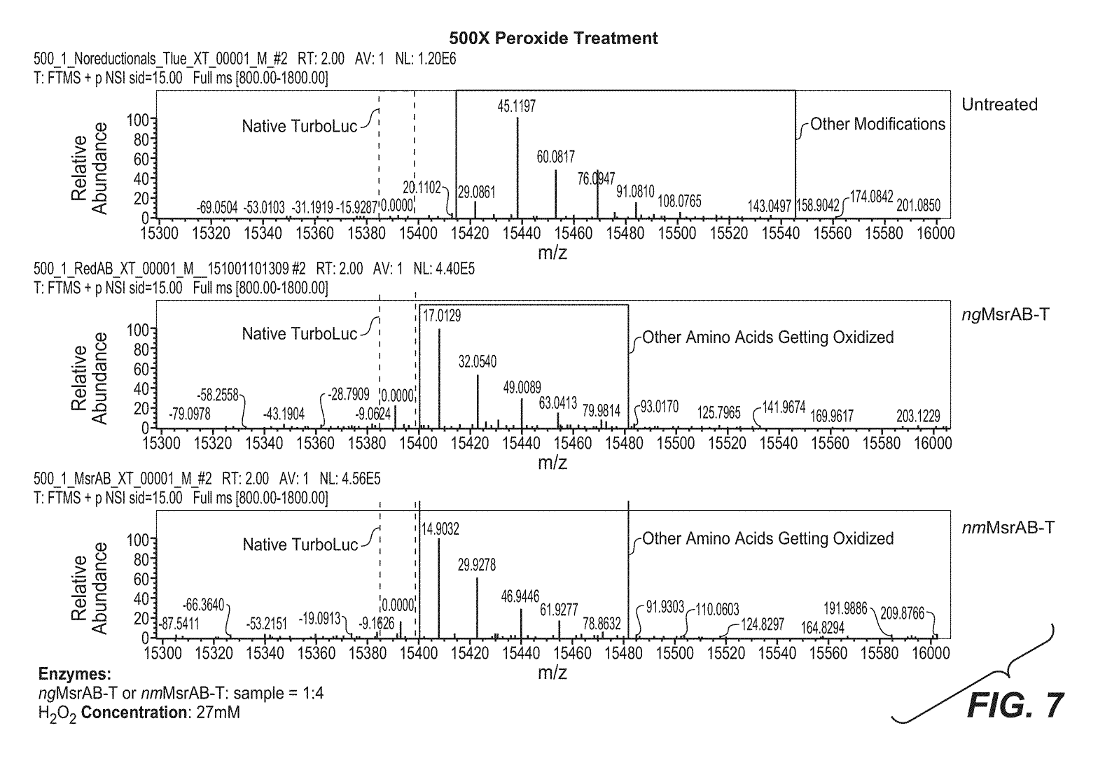

Data on increasingly concentrated hydrogen peroxide treatment are shown for 75× peroxide treatment (4.05 mM H₂O₂, FIG. 5), 100× peroxide treatment (5.4 mM H₂O₂, FIG. 6), and 500× peroxide treatment (27 mM H₂O₂, FIG. 7). Data with 75×-500× peroxide treatment show increases in oxidized methionines in TurboLuc, as well as other modifications to TurboLuc such as oxidation of other amino acids, in oxidized samples that were not reduced. Treatment with ngMsrAB and nmMsrAB counteracted the effect of higher peroxide treatment on methionine oxidation of TurboLuc (for example, see FIG. 5), but treatment with these Msr enzymes did not affect doubly oxidized methionine sulfone or oxidation of other amino acids (for example, see FIG. 7) due to their specificity for methionine. Thus, higher levels of peroxide treatment produced other modifications that could not be counteracted by Msr treatment, such as oxidation of other amino acids, and that led to significant splitting of the signal.

Data presented in FIGS. 4-7 outline successful conditions for reduction of methionine oxidation of TurboLac with Msr treatment using LC-MS for intact protein analysis. In all experiments, there was successful reversal of methionine oxidation of TurboLuc following peroxide treatment with use of the Msr enzymes ngMsrAB and nmMsrAB. The present protocol indicates that the hybrid Msr treatment is a novel means to improve MS analysis of intact proteins due to the reduction of methionine oxidation. Therefore, top-down proteomic analysis can be significantly improved by this novel use of Msr enzymes, such as ngMsrAB and nmMsrAB, to reduce the complexity of data induced by peak splitting of oxidized methionines.

The protocol for top-down analysis of intact proteins was performed as follows. First, 24.6 μl of 0.5M dithiothreitol (DTT) was added to 1.23 mL of 700 μg/ml of whole protein to make a final DIT concentration of 10 mM and incubated for 1 hour at room temperature. Next, 126 μl of 0.5M iodoacetamide (IAM) was added to the mixture to make a final IAM concentration of 50 mM, and the mixture was incubated in dark at room temperature for 20 minutes. Then, 126 μl of 0.5M IAM was added to make a final IAM concentration of 50 mM, and the mixture was further incubated in dark at room temperature for 20 minutes. Next, 54.3 μl of 0.5M DIT was added to bring its final concentration to 20 mM, and the mixture was incubated for 5 minutes at room temperature.

The sample was spun down using a using a 3K MWCO buffer exchange protein concentrator (Fisher, Product #11345402) for 1 hour at 16,000×g. The sample was then buffer exchanged into a slightly basic solution of 50 mM Tris-HCl buffer and appropriate water to create a concentration of 0.333 μg/μL. The preceding procedures were used to create a 500 μl sample of 5 μg/ml for each condition from a 10 μg/ml TurboLuc stock sample for further steps for reduction of the methionines or for a control oxidized sample.

FIG. 16 is a theoretical image presenting the tandem mass spectrometry (MS/MS) spectra for a native and Msr-treated protein. The theoretical native protein shows multiple oxidized (“ox”) peaks, while the Msr-treated protein contains a predominant peak that is non-oxidized. The starred region in the upper panels shows the m/z (mass-to-charge ratio) region selected for MS fragmentation. The lower panels show that the native protein has an MS/MS that is convoluted due to the large number of oxidized precursors that are shifting many of the MS/MS peaks by 16 Da for each oxidized methionine whereas proteins can have dozens of potentially oxidizable methionines. The number of oxidized methionines is indicative of the shift in m/z from the number (N) of 16 Da mass shifts. In contrast, the Msr-treated sample of the same protein shows a cleaner MS/MS spectrum, due to the fact that there is only one dominant precursor non-oxidized peak that was present in the m/z range selected for fragmentation.

### Example 2. Shotgun Proteomic Analysis Coupled with Reversal of Methionine Oxidation

Next, reversal of methionine oxidation via Msr treatment together with shotgun proteomic analysis was investigated using a 6-protein standard (Thermo Scientific Pierce™ 6 Protein Digest, equimolar, LC-MS grade, Product 88342). Analysis of the 6-protein digested standard stored in liquid indicated that it was highly oxidized due to the long-term liquid storage.

The process of shotgun proteomic analysis is outlined in FIG. 8. Proteins were digested and the resulting peptides were separated by LC followed by mass spectrometry.

The Pierce™ 6 Protein Digest, equimolar, LC-MS grade (product #88342) was used as a complex digestion mixture with an estimated methionine concentration for oxidation and subsequent reduction by ngMsrAB or nmMsrAB. In addition, a non-oxidized peptide mixture was set-aside as a control to be reduced by ngMsrAB or nmMsrAB later in the protocol. The following conditions for the oxidation reactions are as follows:

| Sample | | Peroxide | | |
|---|---|---|---|---|
| Volume | Peroxide | Concentration | | |
| (μl) | Volume | (Stock - | Methionine:Peroxide | Water |
| (100 μg) | (μl) | 50.4 mM) | ratio | (μl) |
| 212.7 | 7.5 | 1.25 mM | 1:25 | 79.8 |
| 212.7 | 15 | 2.52 mM | 1:50 | 72.3 |
| 212.7 | 30 | 5.04 mM | 1:100 | 57.3 |

The incubation with hydrogen peroxide was at 37° C. for 1 hour. Then the sample was acidified as needed to obtain a pH˜4 before solid-phase extraction (SPE). SPE was performed using C18 (Sep-Pak vac 1 cc, C-18 cartridges) to clean up the peptide mixture and remove the peroxide using the following protocol. The column was washed with 1 ml of 80% acetonitrile, then equilibrated using 1 ml of 0.1% trifluoroacetic acid (TFA). Sample was then added and washed with 2.2 ml of 0.1% formic acid in water.

The clean peptide sample was collected with 300 μL 80% acetonitrile, and SpeedVac was used to dry the sample. The sample was re-suspended in 700 μl of 0.1% formic acid, and a peptide concentration assay was done to determine the peptide concentration for further steps.

After the peptide concentration was determined, Tris-HCl was added to a final concentration of 50 mM to produce a basic pH and appropriate water to create a concentration of 0.333 μg/μL. Samples were treated with ngMsrAB and nmMsrAB using the following conditions.

| | | | | 5 mM |
|---|---|---|---|---|
| | Sample | | | DTT |
| | Volume | | | (from |
| Samples | (μl) | ngMsrAB-T | nmMsrAB-T | 100 mM |
| (Methionine:Peroxide) | (30 μg) | volume (μl) | volume (μl) | Stock) |
| Control | 90 μl | 7.5 μl | — | 4.9 μl |
| Control | 90 μl | — | 3.41 μl | 4.67 μl |
| 1:25 | 90 μl | 7.5 μl | — | 4.9 μl |
| 1:25 | 90 μl | — | 3.41 μl | 4.67 μl |
| 1:50 | 90 μl | 7.5 μl | — | 4.9 μl |
| 1:50 | 90 μl | — | 3.41 μl | 4.67 μl |
| 1:100 | 90 μl | 7.5 μl | — | 4.9 μl |
| 1:100 | 90 μl | — | 3.41 μl | 4.67 μl |

The peptide mixtures were then injected onto a C18 column (Acclaim PepMap 100 75 μm×100 μm C18) and separated by a gradient of water and acetonitrile in 0.1% formic acid. The resulting liquid flow was infused into the Orbitrap XL mass spectrometer via electrospray ionization utilizing data dependent analysis for collection of MS/MS spectra.

The resulting raw data were processed using Thermo Proteome Discoverer 1.4, and a database search was performed matching theoretical spectra to experimental spectra. For the database search, oxidation was selected as a variable modification or oxidation was not selected at all.

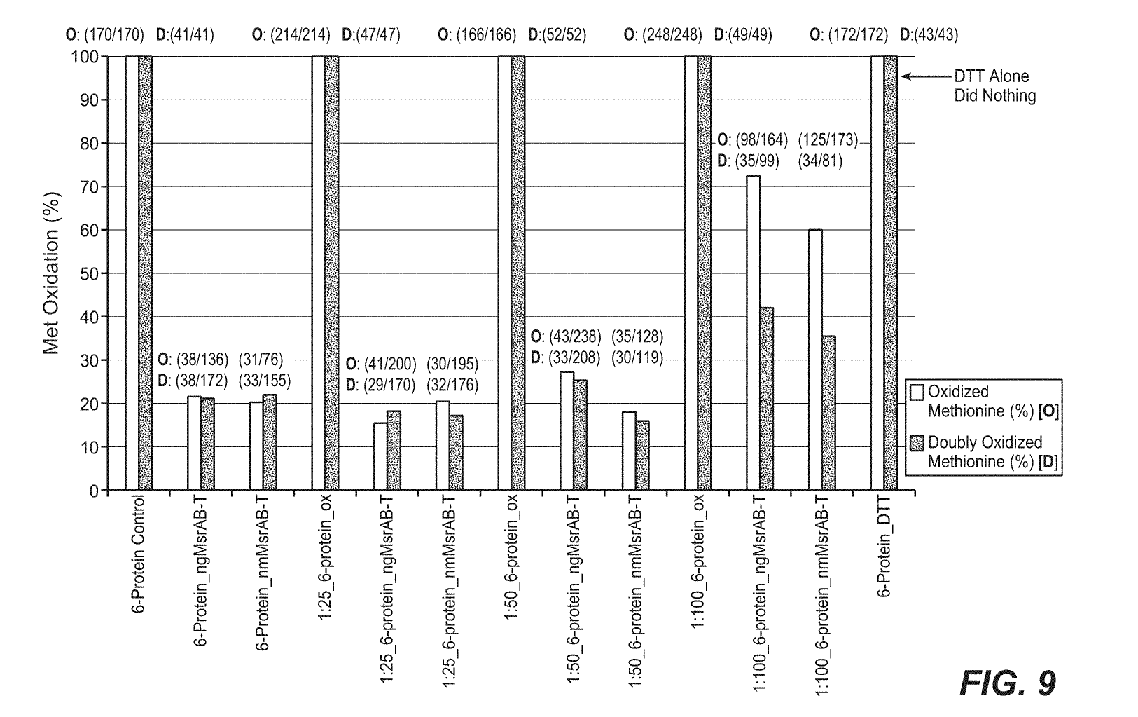

FIG. 9 outlines the percentage of methionine oxidation seen with different methionine:peroxide ratios. Results are presented both for oxidized methionine (O) and doubly oxidized methionine (D). In addition, the ratio of oxidized methionines to total methionines is presented for each condition. For each methionine:peroxide ratio, one sample was included that did not include either ngMsrAB or nmMsrAB and thus provided a measure of 100% methionine oxidation for those conditions. As shown in FIG. 9, data confirmed high levels of methionine oxidation in all samples that were not treated with ngMsrAB or nmMsrAB.

Treatment of samples with ngMsrAB or nmMsrAB at, 1:25, 1:50, and 1:100 methionine:peroxide ratios, as well as unoxidized control samples, were evaluated. Substantial reversal of methionine oxidation was seen with all methionine:peroxide ratios with approximately 80% reversal for all reaction ratios greater than 1:100. In contrast, the DTI control did not show any reversal of methionine oxidation, which confirms the specificity of treatment with ngMsrAB or nmMsrAB. In addition, there was approximately 70% sequence coverage with no loss of coverage based on the methionine reduction.

There was an approximately 20% residual methionine oxidation even following treatment with ngMsrAB or nmMsrAB. This oxidation may be due to the electrospray ionization (ESI) using in the LC analysis, which has been shown to cause oxidation of solution-phase proteins (see Kim K et al., EuPA Open Proteomics 8:40-47 (2015)).

These results with shotgun proteomics analysis following Msr treatment confirm the ability to reproducibly reduce methionine oxidation of a highly oxidized sample. Critically, this reduction of methionine oxidation occurred without sample loss. Normally, methionine oxidation is included as a permitted variable modification, compromising database searches following shotgun proteomic analysis. Therefore, treatment with ngMsrAB or nmMsrAB is a novel means to simplify and improve database searching for large shotgun discovery proteomics experiments by reducing the data variability induced by methionine oxidation.

### Example 3. Targeted Protein Analysis of Methionine Reduction by Parallel Reaction Monitoring

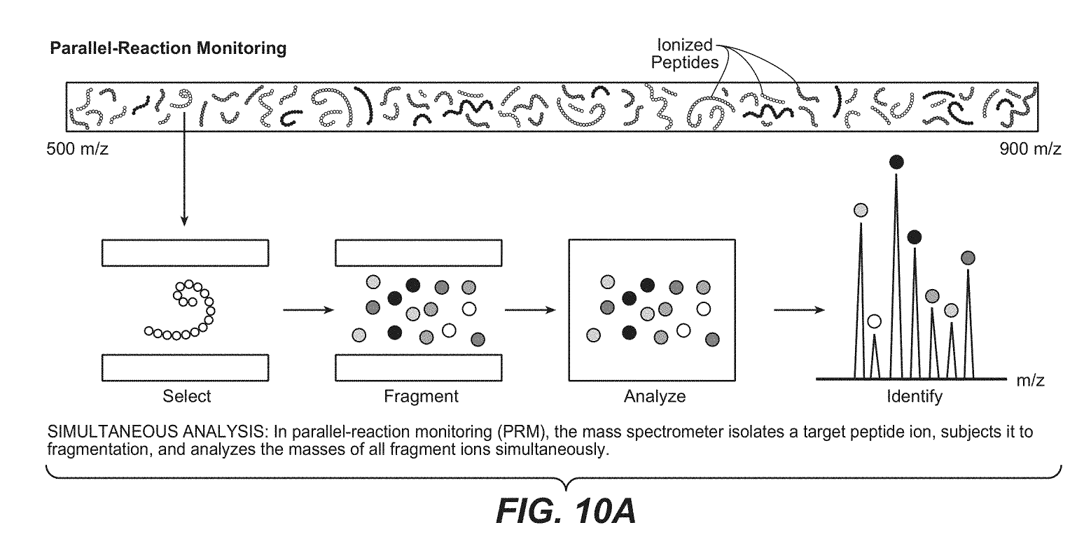

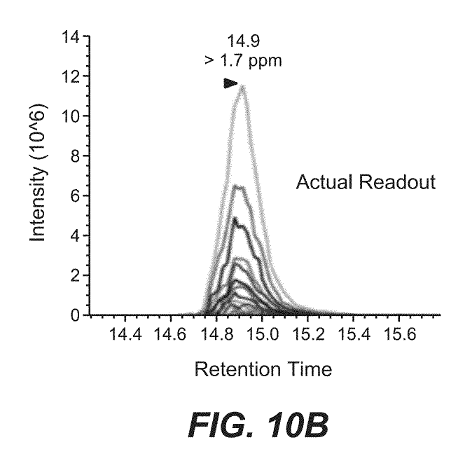

Based on the results using shotgun proteomics, targeted protein analysis following reduction of methionine oxidation was investigated using parallel reaction monitoring (PRM). The basis of PRM is a targeted proteomics strategy where all fragmentation products of a target peptide are simultaneously monitored under conditions that offer high resolution and mass accuracy, as outlined in FIG. 10A (see also Peterson A C, et al., Molecular & Cellular Proteomics 11:1475-1488 (2012)). In PRM, the mass spectrometer isolates a target peptide ion and subjects it to fragmentation, followed by simultaneous analysis of the masses of all fragment ions. FIG. 10B shows representative experimental data wherein intensity is plotted against retention time to give a readout of the masses of all fragment ions.

Quantitative PRM analysis was done using the Q Exactive HF Hybrid Quadrupole-Orbitrap Mass Spectrometer (QE-HF) (Thermo Fisher). The sample used to validate methionine reduction by Msr treatment was a 3-peptide mix prepared from commercially prepared peptide standards that included SEQ ID Nos: 4, 6, and 8 as listed in the following table.

| | Sequence and post-translational |
|---|---|
| SEQ ID Nos. | modifications |
| SEQ ID No: 4 | RTTSFAESCKPVQQPSAFGSMKV |
| | Wherein C indicates a carbamidomethyl |
| | C, and |
| | Wherein M indicates an oxidized M |
| SEQ ID No: 5 | RTTSFAESCKPVQQPSAFGSMKV |
| | Wherein C indicates a carbamidomethyl |
| | C |
| SEQ ID No: 6 | RTTSFAESCKPVQQPSAFGSMKV |
| | Wherein S indicates a phosphorylated |
| | S, |
| | Wherein C indicates a carbamidomethyl |
| | C, and |
| | Wherein M indicates an oxidized M |
| SEQ ID No: 7 | RTTSFAESCKPVQQPSAFGSMKV |
| | Wherein S indicates a phosphorylated |
| | S, and |
| | Wherein C indicates a carbamidomethyl |
| | C |
| SEQ ID No: 8 | RTESITATSPASMVGGKPGSFRV |
| | Wherein S indicates a phosphorylated |
| | S, and |
| | Wherein M indicates an oxidized M |
| SEQ ID No: 9 | RTESITATSPASMVGGKPGSFRV |
| | Wherein S indicates a phosphorylated S |

The three oxidized heavy peptides including both phosphorylated and non-phosphorylated peptides (100 fmol/μl) were mixed with a digested 6-protein mix background matrix (40 ng/μl, as described in Example 2) and IM tetraethylammonium bromide (TEAB) to a concentration of 2 mM to produce a slightly basic pH.

The amount of sample, enzyme, water, and DTT added are shown below for each sample reacted with ngMsrAB or nmMsrAB:

| | | | | 5 mM DTT |
|---|---|---|---|---|
| | Sample | Enzyme | | from 100 mM |
| | Volume | Volume | | stock |
| Sample | (μl) | (μl) | Water | (μl) |
| Sample mix | 100 | — | 7 μl | — |
| Control | | | | |
| Sample mix | 100 | 1 | — | 5.05 |
| with ngMsrAB, 1 | | | | |
| Sample mix | 100 | 2.27 | — | 5.11 |
| with nmMsrAB, 1 | | | | |
| Sample mix | 100 | 1 | — | 5.05 |
| with | | | | |
| ngMsrAB15 | | | | |
| Sample mix | 100 | 2.27 | — | 5.11 |
| with nmMsrAB, | | | | |
| 15 | | | | |

The ratio of enzyme:protein was 1:4, and DTT had a final concentration of 5 mM for all reactions. Samples were incubated at 37° C. for 2 hours with gentle vortexing throughout. The resulting peptide mixture was then injected onto a C18 column (Easy-Spray PepMap C18, 3 μm, 75 μm×150 cm) and separated by a gradient of water and acetonitrile in 0.1% formic acid.

The resulting liquid flow was infused into the Q Exactive HF mass spectrometer via electrospray ionization utilizing parallel reaction monitoring for collection of targeted spectra. The resulting raw data were processed and visualized using Skyline 3.1.0.7382 open source PRM analysis software.

FIG. 11A shows the disappearance of methionine oxidation of one peptide in the mix (SEQ ID No: 4) following treatment with ngMsrAB or nmMsrAB, while FIG. 11B shows the appearance of the peptide reduced at the methionine residue with treatment (SEQ ID No: 5). In FIGS. 11A and 11B, the bar graph areas show the cumulative area under the curve of individual b-ion and y-ion fragments (shown with different gray-scale shading) following treatment with ngMsrAB or nmMsrAB. These data indicate that greater than 90% reduction of an oxidized methionine is produced by treatment with the Msrs ngMsrAB or nmMsrAB, with a corresponding increase in the peptide with a reduced methionine. Note that oxidation of the cysteine residue of SEQ ID No: 4 and 5 was not affected by treatment with Msrs, as ngMsrAB or nmMsrAB are specific for reduction of oxidized methionines.

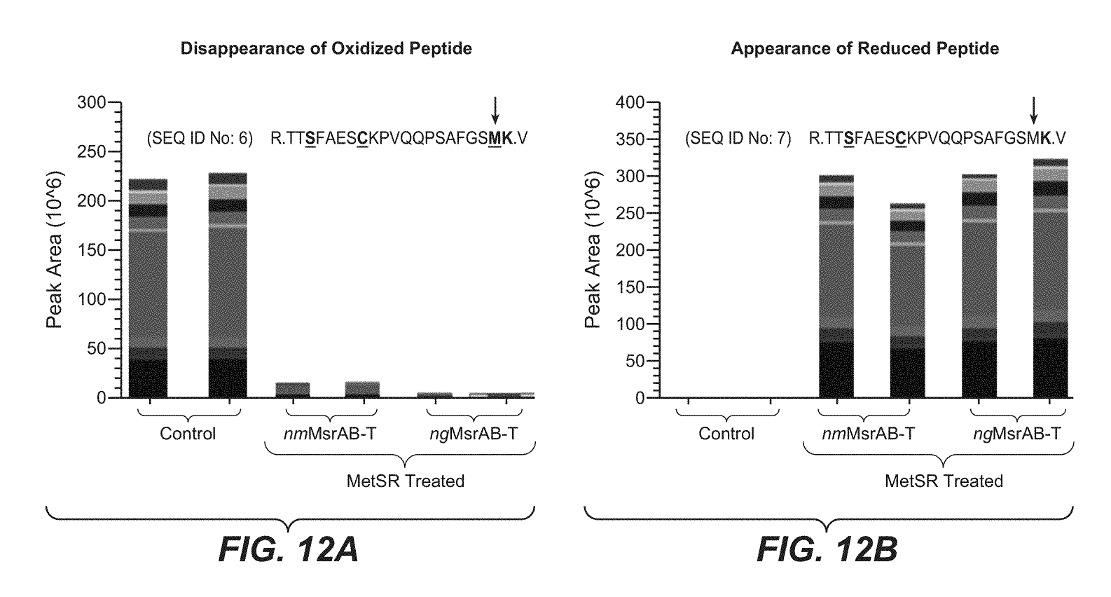

Next, Msr treatment was performed on the phosphoprotein SEQ ID No: 6, wherein there is a phosphorylated serine residue in addition to an oxidized methionine and cysteine residue. Treatment of SEQ ID No: 6 with ngMsrAB or nmMsrABT produced a disappearance of SEQ ID No: 6 (as shown in FIG. 12A) and the appearance of the phosphopeptide SEQ ID No: 7 that retained phosphorylation of the serine and oxidation of cysteine residues, but had reduction of the methionine residue (as shown in FIG. 12B). These data indicate that Msr treatment with ngMsrAB or nmMsrAB can produce a quantitative reduction of methionine oxidation with high specificity such that other post-translational modifications (including cysteine oxidation and serine phosphorylation) are maintained.

The specificity of Msr treatment was further confirmed with the third peptide in the 3-peptide mix, SEQ ID No: 8, which is also a phosphopeptide (i.e., the second phosphopeptide). SEQ ID No: 8 has both a phosphorylated serine and an oxidized methionine residue. As shown in FIG. 13A, Msr treatment with ngMsrAB or nmMsrAB produced a disappearance of SEQ ID No: 8. In contrast, FIG. 13B shows that these treatments caused the appearance of a reduced phophopeptide that lacked an oxidated methionine but maintained the serine phosphorylation (SEQ ID No: 9). These data confirm that Msr treatment with ngMsrAB or nmMsrAB is specific for reduction of oxidized methionine residues while maintaining the serine phosphorylation of treated peptides.

As noted above, FIG. 16 is a theoretical image presenting the tandem mass spectrometry (MS/MS) spectra for a native and Msr-treated protein. The theoretical native protein shows multiple oxidized (“ox”) peaks, while the Msr-treated protein contains a predominant peak that is non-oxidized. The starred region in the upper panels shows the m/z (mass-to-charge ratio) region selected for MS fragmentation. The lower panels show that the native protein has an MS/MS that is convoluted due to the large number of oxidized precursors that are shifting many of the MS/MS peaks by 16 Daltons (Da) for each oxidized methionine whereas proteins can have dozens of potentially oxidizable methionines. The number of oxidized methionines is indicative of the shift in m/z from the number (N) of 16 Da mass shifts. In contrast, the Msr-treated sample of the same protein shows a cleaner MS/MS spectrum, due to the fact that there is only one dominant precursor non-oxidized peak that was present in the m/z range selected for fragmentation.

The data in FIGS. 11, 12, and 13 indicate that Msr treatment with ngMsrAB or nmMsrAB can overcome variable oxidation of methionine residues in targeted analysis.

The results described herein demonstrate that treatment with methionine sulfoxide reductase can simplify mass spectrometry analysis by reducing the occurrence of oxidized methionine, and the resulting analysis complexity.

TABLE OF SEQUENCES

| SEQ | | |
|---|---|---|
| ID NO | Description | Sequence |
| 1 | MsrAB from Neisseria | HHHHHHSSGL VPRGSHMWEL QLALGACSPK IVDAGAATVP |
| | gonorrhoeae, with His | HTLSTLKTAD NRPASVYLKK DKPTLIKFWA SWCPLCLSEL |
| | tag/WQ protease site | GQAEKWAQDA KFSSANLITV ASPGFLHEKK DGEFQKWYAG |
| | | LNYPKLPVVT DNGGTIAQNL NISVYPSWAL IGKDGDVQRI |
| | | VKGSINEAQA LALIRNPNAD LGSLKHSFYK PDTQKKDSAI |
| | | MNTRTIYLAG GCFWGLEAYF QRIDGVVDAV SGYANGNTEN |
| | | PSYEDVSYRH TGHAETVKVT YDADKLSLDD ILQYYFRVVD |
| | | PTSLNKQGND TGTQYRSGVY YTDPAEKAVI AAALKREQQK |
| | | YQLPLVVENE PLKNFYDAEE YHQDYLIKNP NGYCHIDIRK |
| | | ADEPLPGKTK AAPQGKGFDA ATYKKPSDAE LKRTLTEEQY |
| | | QVTQNSATEY AFSHEYDHLF KPGIYVDVVS GEPLFSSADK |
| | | YDSGCGWPSF TRPIDAKSVT EHDDFSFNMR RTEVRSRAAD |
| | | SHLGHVFPDG PRDKGGLRYC INGASLKFIP LEQMDAAGYG |
| | | ALKGKVK |
| 10 | MsrAB from Neisseria | LALGACSPKI VDAGAATVPH TLSTLKTADN RPASVYLKKD |
| | gonorrhoeae | KPTLIKFWAS WCPLCLSELG QAEKWAQDAK FSSANLITVA |
| | | SPGFLHEKKD GEFQKWYAGL NYPKLPVVTD NGGTIAQNLN |
| | | ISVYPSWALI GKDGDVQRIV KGSINEAQAL ALIRNPNADL |
| | | GSLKHSFYKP DTQKKDSAIM NTRTIYLAGG CFWGLEAYFQ |
| | | RIDGVVDAVS GYANGNTENP SYEDVSYRHT GHAETVKVTY |
| | | DADKLSLDDI LQYYFRVVDP TSLNKQGNDT GTQYRSGVYY |
| | | TDPAEKAVIA AALKREQQKY QLPLVVENEP LKNFYDAEEY |
| | | HQDYLIKNPN GYCHIDIRKA DEPLPGKTKA APQGKGFDAA |
| | | TYKKPSDAEL KRTLTEEQYQ VTQNSATEYA FSHEYDHLFK |
| | | PGIYVDVVSG EPLFSSADKY DSGCGWPSFT RPIDAKSVTE |
| | | HDDFSFNMRR TEVRSRAADS HLGHVFPDGP RDKGGLRYCI |
| | | NGASLKFIPL EQMDAAGYGA LKGKVK |
| 2 | MsrAB from Neisseria | HHHHHHSSGL VPRGSHMWEL QLALGACSPK IVDAGAATVP |
| | meningitides, with His | HTLSTLKTAD NRPASVYLKK DKPTLIKFWA SWCPLCLSEL |
| | tag/WQ protease site | GQTEKWAQDA KFSSANLITV ASPGFLHEKK DGDFQKWYAG |
| | | LNYPKLPVVT DNGGTIAQSL NISVYPSWAL IGKDSDVQRI |
| | | VKGSINEAQA LALIRDPNAD LGSLKHSFYK PDTQKKDSKI |
| | | MNTRTIYLAG GCFWGLEAYF QRIDGVVDAV SGYANGNTKN |
| | | PSYEDVSYRH TGHAETVKVT YDADKLSLDD ILQYFFRVVD |
| | | PTSLNKQGND TGTQYRSGVY YTDPAEKAVI AAALKREQQK |
| | | YQLPLVVENE PLKNFYDAEE YHQDYLIKNP NGYCHIDIRK |
| | | ADEPLPGKTK TAPQGKGFDA ATYKKPSDAE LKRTLTEEQY |
| | | QVTQNSATEY AFSHEYDHLF KPGIYVDVVS GEPLFSSADK |
| | | YDSGCGWPSF TRPIDAKSVT EHDDFSYNMR RTEVRSHAAD |
| | | SHLGHVFPDG PRDKGGLRYC INGASLKFIP LEQMDAAGYG |
| | | ALKGKVK |
| 11 | MsrAB from Neisseria | LALGACSPKI VDAGAATVPH TLSTLKTADN RPASVYLKKD |
| | meningitides | KPTLIKFWAS WCPLCLSELG QTEKNAQDAK FSSANLITVA |
| | | SPGFLHEKKD GDFQKWYAGL NYPKLPVVTD NGGTIAQSLN |
| | | ISVYPSWALI GKDSDVQRIV KGSINEAQAL ALIRDPNADL |
| | | GSLKHSFYKP DTQKKDSKIM NTRTIYLAGG CFWGLEAYFQ |
| | | RIDGVVDAVS GYANGNTKNP SYEDVSYRHT GHAETVKVTY |
| | | DADKLSLDDI LQYFFRVVDP TSLNKQGNDT GTQYRSGVYY |
| | | TDPAEKAVIA AALKREQQKY QLPLVVENEP LKNFYDAEEY |
| | | HQDYLIKNPN GYCHIDIRKA DEPLPGKTKT APQGKGFDAA |
| | | TYKKPSDAEL KRTLTEEQYQ VTQNSATEYA FSHEYDHLFK |
| | | PGIYVDVVSG EPLFSSADKY DSGCGWPSFT RPIDAKSVTE |
| | | HDDFSYNMRR TEVRSHAADS HLGHVFPDGP RDKGGLRYCI |
| | | NGASLKFIPL EQMDAAGYGA LKGKVK |
| 12 | Methionine sulfoxide | FALGACSPKI ADAEAATVPH TLSTLKTADN RPADVYLKKD |
| | reductase, Neisseria | KPTLIKFWAS WCPLCLSELG QTEKWAQDAK FGSANLITVA |
| | lactamica | SPGFLHEKKD GDFQKWYAGL NYPKLPVVTD NGGTIAQSLN |
| | | ISVYPSWALI GKDGDVQRIV KGSINEAQAL ALIRDPNADL |
| | | GSLKHSFYKP DTQKKDSKIM NTRTIYLAGG CFWGLEAYFQ |
| | | RIDGVVDAVS GYANGNTKNP SYEDVSYRHT GHAETVKVTY |
| | | DADKLSLDDI LQYYFRVVDP TSLNKQGNDT GTQYRSGVYY |
| | | TDPAEKAVIA AALKREQQKY KLPLVVENEP LKNFYDAEEY |
| | | HQDYLIKNPN GYCHIDIRKA DEPLPGKTKT APQGKGFDAA |
| | | TYKKPSDAEL KRTLTEEQYQ VTQKSATEYA FSHEYDHLFK |
| | | PGIYVDVVSG EPLFSSADKY DSGCGWPSFT RPIDAKSVTE |
| | | HDDFSFNMRR TEVRSHAADS HLGHVFPDGP RDKGGLRYCI |
| | | NGASLKFIPL EQMDAAGYGA LKGKVK |
| 13 | Methionine sulfoxide | FALGACSPKI ADAEAATVPH TLSTLKTADN RPADVYLKKD |
| | reductase, Neisseria | KPTLIKFWAS WCPLCLSELG QTEKWAODAK FGSANLITVA |
| | polysaccharea | SPGFLHEKKD GDFQKWYAGL NYPKLPVVTD NGGTIAQSLN |
| | | ISVYPSWALI GKDGDVQRIV KGSINEAQAL ALIRDPNADL |
| | | GSLKHSFYKP DTQKKDSKIM NTRTIYLAGG CFWGLEAYFQ |
| | | RIDGVVDAVS GYANGNTKNP SYEDVSYRHT GHAETVKVTY |
| | | DADKLSLDDI LQYYFRVVDP TSLNKQGNDT GTQYRSGVYY |
| | | TDPAEKTVIA AALKREQQKY KLPLVVENEP LKNFYDAEEY |
| | | HQDYLIKNPN GYCHIDIRKA DEPLPGKTKT APQGKGFDAA |
| | | TYKKPSDAEL KRTLTEEQYQ VTQHSATEYA FSHEYDHLFK |
| | | PGIYVDVVSG EPLFSSADKY DSGCGWPSFT RPIDAKSVTE |
| | | HDDFSYNMRR TEVRSHAADS HLGHVFPDGP RDKGGLRYCI |
| | | NGASLKFIPL EQMDAAGYGA LKGKVK |
| 14 | Methionine sulfoxide | FALGACSPKT ADAGAATVPH TLSTLKTADN RPAGVYLKKD |
| | reductase, Neisseria | KPTLIKFWAS WCPLCLSELG QTEKWAQDAK FGSANLITVA |
| | flavescens | SPGFLHEKKD GDFQKWYAGL NYPKLPVVTD NGGTIAQSLN |
| | | ISVYPSWALI GKDGDVQRIV KGSINEAQAL ALIRDPNADL |
| | | GSLKHSFYKP DTQKKDSKIM NTRTIYLAGG CFWGLEAYFQ |
| | | RIDGVVDAVS GYANGNTKNP SYEDVSYRHT GHAETVKVTY |
| | | DADRLSLDDI LQYYFRVVDP TSLNKQGNDT GTQYRSGVYY |
| | | TDPAEKAVIA AALKREQQKY KLPLVVENEP LKNFYDAEEY |
| | | HQDYLIKNPN GYCHIDIRKA DEPLAGKTQT APQGKGFDAA |
| | | TYKKPSDAEL KRTLTEEQYQ VTQHSATEYA FSHEYDHLFK |
| | | PGIYVDVVSG EPLFSSADKY DSGCGWPSFT RPIDAKSVTE |
| | | HNDFSYNMRR TEVRSHAADS HLGHVFPDGP RDKGGLRYCI |
| | | NGASLKFIPL EQMDAAGYGA LKGKVK |
| 15 | Methionine sulfoxide | FALGACSPKI ADAEAATVPH TLSTLKTADN RPASVYLKKD |
| | reductase, Neisseria | KPTLIKFWAS WCPLCLSELG QTEKWAQDKR FSSANLITVA |
| | sicca | SPGFLHEKKD GDFQKWYAGL NYPKLPVVTD NGGTIAQSLN |
| | | ISVYPSWALI GKDGDVQRIV KGSINEAQAL ALIRDPNADL |
| | | GRLKNAFYKP DTQKKDSKIM NTRTIYLAGG CFWGLEAYFQ |
| | | RIDGVVDAVS GYANGKTKNP SYEDVSYRDT GHAETVKVTY |
| | | DADKLSLDDI LQYYFRVVDP TSLNKQGNDT GTQYRSGVYY |
| | | TDPAEKAVIA AALKREQQKY KQPLVVENEP LKNFYDAEEY |
| | | HQDYLIKNPN GYCHIDIRKA DEPLPGKTKA APQGKGFDAA |
| | | TYKKPSDAEL KRILTEEQYQ VTQKSATEYA FSHEYDHLFK |
| | | PGIYVDVVSG EPLFSSADKF DSGCGWPSFT RPINAAAVTE |
| | | HDDFSYNMRR TEVRSHAADS HLGHVFPDGP KDKGGLRYCI |
| | | NGASLKFIPL EQMDAAGYGA LKGKVK |
| 16 | Methionine sulfoxide | FALGACSPKI ADAEAATVPH TLSTLKTADN RPASVYLRKD |
| | reductase, Neisseria | KPTLIKFWAS WCPLCLSELG QTEKWAQDKR FSSANLITVA |
| | macacae | SPGFLHEKKD GDFQKWYAGL NYPKLPVVTD NGGTIAQSLN |
| | | ISVYPSWALI GKDGDVQRIV KGSINEAQAL ALIRDPNADL |
| | | GRLKNAFYKP DTQKKDSKIM NTRTIYLAGG CFWGLEAYFQ |
| | | RIDGVVDAVS GYANGKTKNP SYEDVSYRDT GHAETVKVTY |
| | | DADKLSLDDI LQYYFRVVDP TSLNKQGNDT GTQYRSGVYY |
| | | TDPAEKAVIA AALKREQQKY KQPLVVENEP LKNFYDAEEY |
| | | HQDYLIKNPN GYCHIDIRKA DEPLPGKTKA APQGKGFDAA |
| | | TYKKPSDAEL KRILTEEQYQ VTQKSATEYA FSHEYDHLFK |
| | | PGIYVDVVSG EPLFSSADKF DSGCGWPSFT RPINAAAVTE |
| | | HDDFSYNMRR TEVRSHAADS HLGHVFPDGP KDKGGLRYCI |
| | | NGASLKFIPL EQMDAAGYGA LKGKVK |
| 17 | Methionine sulfoxide | FALGACSPKI ADAEAATVPH TLSTLKTADN RPASVYLKKD |
| | reductase, Neisseria | KPTLIKFWAS WCPLCLSELG QTEKWAQDKR FSSANLITVA |
| | mucosa | SPGFLHEKKD GDFQKWYAGL NYPKLPVVTD NGGTIAQSLN |
| | | ISVYPSWALI GKDGDVQRIV KGSINEAQAL ALIRDPNADL |
| | | GRLKNAFYKP DTQKKDSKIM NTRTIYLAGG CFWGLEAYFQ |
| | | RIDGVVDAVS GYANGKTKNP SYEDVSYRDT GHAETVKVTY |
| | | DADKLSLDDI LQYYFRVVDP TSLNKQGNDT GTQYRSGVYY |
| | | TDPAEKAVIA AALKREQQKY KQPLVIENEP LKNFYDAEEY |
| | | HQDYLIKNPN GYCHIDIRKA DEPLPGKTKA APQGKGFDAA |
| | | TYKKPSDAEL KRILTEEQYQ VTQKSATEYA FSHEYDHLFK |
| | | PGIYVDVVSG EPLFSSADKF DSGCGWPSFT RPINAAAVTE |
| | | HDDFTYNMRR TEVRSHAADS HLGHVFADGP QDKGGLRYCI |
| | | NGASLKFIPL EQMDAAGYGA LKGKVK |
| 18 | Methionine sulfoxide | LALGACSSKI MDTEAATVPQ ALSSLKTPDN RPASVFLKKD |
| | reductase, Neisseria | KPTLIKFWAS WCPLCLSELG QTEKWAQDTK FGSANLITVA |
| | flavescens | SPGFLHEKKD GDFQKWYAGL NYPKLPVVTD NGGTIAQSLN |
| | | ISVYPSWALI GKDGDVQRIV KGSINEAQAL ALIRDPNADL |
| | | GRLKNSFYKP DTQKKDSAIM NTRTIYLAGG CFWGLEAYFQ |
| | | RIDGVVDAVS GYANGKTENP SYEDVSYRDT GHAETVKVTY |
| | | DADKLSLDDI LQYFFRVVDP TSLNKQGNDT GTQYRSGVYY |
| | | TDPAEKAVIA AALKREQQKY KQPLVVENEP LKNFYDAEEY |
| | | HQDYLIKNPN GYCHIDIRKA DEPLPGKTKA APQGKGFDAA |
| | | TYKKPGAAEL KRLLTEEQYQ VTQNSATEYA FSHEYDHLFK |
| | | PGIYVDVVSG EPLFSSADKF DSGCGWPSFT HPINASAVTE |
| | | HDDFSYNMRR TEVRSHAADS HLGHVFPDGP KDKGGLRYCI |
| | | NGASLKFIPL EQMDAAGYGA LKGKVK |
| 19 | Methionine sulfoxide | MIMRRLLTPR NLLLLVLLAV MFWSFYSGAS PSHGTPPASA |
| | reductase, Lautropia | DKAATAQGGG AAGAAQASDG APEQPVGLPL AYLQKLKDVA |
| | mirabilis | DKPATTYIKP GRPTLVKFWA SWCPLCLSEL ADTNAWATDE |
| | | RFSSAVNLVT LASPGFLHEK PQADFVTWYG GLDYPAMPVL |
| | | LDVGGLLARQ LGVRVYPSWV LLDADGGVAR VVRGRLSEAQ |
| | | ALALIEDPEA DLARLAQAER ASFYQPDSQK SSKVMNTKTI |
| | | YLAGGCFWGV EAYFQRIPGV VDAVSGYANG RTQNPSYEDV |
| | | IRGAGHAETV KVTYDADRLS LADILQYYFR IIDPTSLNKQ |
| | | GNDRGAQYRT GVYYTDAADK ATIQQALDAL QQKYSRPLVV |
| | | ENLPLQNFYE AEEYHQDYLA KNPNGYCHID VRKADEPLPG |
| | | KPAGNPPAAA AVGRGFDVAS YRKASDAELK QRLSAEQYRV |
| | | TQQSGTERAF THEYDHLFAP GIYVDVVSGQ PLFSSKDKFD |
| | | SGCGWPSFTR PIQPSAVTEH EDLSYNMRRV EVRSQAADSH |
| | | LGHVFPDGPR DKGGLRYCIN GASLRFIPLE KMAEEGYGNL |
| | | VDAVK |
| 20 | Methionine sulfoxide | MKNPRQTLCS LIACVLFAGA VAPLPVLADA HASRAEAPLP |
| | reductase, | HQLQQRLLAL KDPRDQPAAD YLDQSKPTLI KFWASWCPLC |
| | Cardiobacierium | LATLEETQAW RGDKAFAGVN LVTIASPDHL GENDEATFKE |
| | hominis | WYRGLDYPNL PVLVNNGGDI ARDIGVAVYP SWALLDKNGN |
| | | VARVIKGHIN REQALALLAN PQAELAQPAQ KFYKPKPKGA |
| | | TNMNTKTIHL AGGCFWGLEA YFERIPGVVD AVSGYANGKT |
| | | KNPSYEDVSH RGTGHAETVK VTYDPERISL DDLLRYYFRV |
| | | VDPTSLNQQG NDRGVQYRSG VYYTDPAERA TIEKAFAEEQ |
| | | KKHQKPLVVE NLPLDNFYEA EEYHQDYLAK NPNGYCHIDI |
| | | RKADIPLEKP AATAPAPAQT DANGEPVIDA AKYHKPDAAE |
| | | LKQKLDAQAY EVTQNSATER AFSHEYDHLF APGLYVDVVS |
| | | GEPLFSSADK FQSGCGWPSF TKPINRAVVT EHDDTSYNMH |
| | | RTEIRSRVAD AHLGHVFPDG PKDKGGLRYC INGASLKFIP |
| | | LAEMEKAGYG DLVDAVKKGE KL |
| 21 | Methionine sulfoxide | MKNPRQTLCS LIACVLFAGA VAPLPVLADA HASRAEAPLP |
| | reductase, | HQLQQRLLAL KDPRDKPAAD YLDQSKPTLI KFWASWCPLC |
| | Gammaproteobacteria | LATLEETQAW RGDKAFAGVN LVTIASPDHL GENDEATFKE |
| | | WYRGLDYPNL PVLVNNGGDI ARDIGVAVYP SWALLDKNGN |
| | | VARVIKGHIN REQALALLAN PQAELAQPAQ KFYKPKPKGA |
| | | TNMNTKTIHL AGGCFWGLEA YFERIPGVVD AVSGYANGKT |
| | | KNPSYEDVSH RGTGHAETVK VTYDPERISL DDILRYYFRV |
| | | VDPTSLNQQG NDRGVQYRSG VYYTDPAERA TIEKAFAEEQ |
| | | KKHQKPLVVE NLPLDNFYEA EEYHQDYLAK NPNGYCHIDI |
| | | RKADIPLEKP AATAPAPAQT DANGEPVIDA TKYHKPDAAE |
| | | LKKKLDAQAY EVTQNSATER AFSHEYDHLF APGLYVDVVS |
| | | GEPLFSSADK FQSGCGWPSF TKPINRAVVT EHDDTSYNMH |
| | | RTEIRSRVAD AHLGHVFPDG PKDKGGLRYC INGASLKFIP |
| | | LAEMAKAGYG DLVDAVKKGE KL |
| 22 | Methionine sulfoxide | MKSPLAKANK PNFFQQLTQL QPVTNGSSNM QFNNNRPTLV |
| | reductase, | KLWASWCPLC LSELELTQSW ANDPDFAQVN LTTLASPGVL |
| | Marinospirillum | GELSLEEFKQ WYAGLDYPDL PLQLDPSGEL VKKLGVQVYP |
| | insulare | SWAVLDAQGN LQRVVKGSIN KAQALALIAN PEADLKQLQT |
| | | TFYQPKQPAQ ALPINTQSVY LAGGCFWGVE GYFERIDGVV |
| | | DAVSGYANGR TENPSYEDVI YRNTGHAETV KVTYNSDKLS |
| | | LDDILVYFFR IIDPTSLNKQ GNDRGTQYRT GIYTTDPAEQ |
| | | RLVATALARL EEEYTQPILV ENLPLSGFYE AEEYHQDYLL |
| | | KNPNGYCHVD LNKADIPLPN QLTNQSTDKN TPKPFDPNNF |
| | | QKPDTASLKQ RLTSEQFHVT QNNGTERAFT HEYDDLFEPG |
| | | LYVDIVSGEP LFSSKDKYQA GCGWPSFVKP IEENALVEVV |
| | | DTSYNMRRIE VRSRLADSHL GHVFPDGPKD RGGLRYCING |
| | | ASLKFIPLAE MQAQGYGDWQ ALIN |
| 23 | methionine sulfoxide | MPFLYFLRTI ILGIMALYSS TLFAQTINFN ALKDINNQKA |
| | reductase, Pelistega | NFYIKNNKPT VVKFWASWCP LCLGELEQTE QWVQDKDFAM |
| | indica | VNMVTLASPG YLGEKKAADF SQWALSLPYK KLPILIDTEQ |
| | | TIAKSLNIRV YPSWVLLDSN GQLVKVVKGT LSKEQLLGVI |
| | | KNPDAPIQKA STTFYKADTN SEHKKPIRTE TIYLAGGCFW |
| | | GLEGYFQRIP GVIDAVSGYA NGNTQNPSYE DVVYRHTGHA |
| | | ETVKVTYDID KLSFADILEY YFRVIDPTSL NQQGNDKGTQ |
| | | YRTGIYYTKA DYQPLIAEAI KKEQTKYKKP IVVENKPLAN |
| | | FYPAEEYHQD YLLKNPNGYC HIDLNKADEP LSTPSPKGFN |
| | | MKEYKKPSQS ELRQRLTPEQ YRVTQESGTE YAFSHEYDEL |
| | | FAPGLYVDIV SGQPLFSSDD KFNSHCGWPS FTQPIEKTVV |
| | | TEHKDFSHNM YRIEVRSQAA DSHLGHVFPD GPADRGGLRY |
| | | CINGASLRFI PYADLDKEGY GEWKDKIKQK |
| 24 | methionine sulfoxide | MKKIFALCVT LGIALVTLAF AKLPNSSTDK ATQGADDKAF |
| | reductase, Basilea | TYLLSLDDIH QQPAKQLIDT NRPTLVKLWA SWCSSCLSEL |
| | psittacipulmonis | DEVEAWSKDK RFKAINFVTV VSPSLYSEKN KDDFTKWFLS |
| | | LDYPQTKVLL DTKGTLSRTL NIRAYPSWAL FDEKGHLVRV |
| | | IKGSISKVQA LALIDNPQAD LKSVQEKANR VTKKEVIDPM |
| | | YQKTIYLAGG CFWGVEAYFE RIDGVIDAVS GYANGRTENP |
| | | KYEDVIYRHT GHAETVKVTF DTRRLSLADI LQYYFRVIDP |
| | | TSLNKQGNDR GTQYRTGVYY TDEKDKAVID AALANEQKKY |
| | | TKPLVVENLP LRNFYLAEDY HQDYLKKNPN GYCHIDISLA |
| | | DRPLERGTNI DKPVRFWETY EKPSDNELRQ QLSNEQYRIT |
| | | QKNGTEYAFS HAYDHLFEPG LYVDIVSGEP LFTSTDKYDS |
| | | GCGWPSFTQP IQAQAITEHE DLSYNMRRIE VRSRYADSHL |
| | | GHVFPDGPSD KGGLRYCING ASLRFIPLEQ MAAEGYAEFI |
| | | PLIKKP |
| 25 | methionine sulfoxide | MVQKIPHFFL SILFLTLTVL SLPAQSFSFS TKQHLGPRLE |
| | reductase, Oligella | KLNDVQGVKA TEFLQTSRPT LVKFWASWCP LCLATLEETR |
| | ureolytica | DWRLDPDFSN TDIVTLASPG YLKEQSPKDF RQWYQGVNIE |
| | | HLPVLVNDGG DLTREIGVSV YPSWALLDAQ GRLQRVIKGH |
| | | ITKEQALGLI ADKDFDIQRS APTFYRPSTD TAQQQKDKSN |
| | | LMNSKEIYLA GGCFWGVEAY FERIPGVLDA ISGYANGNTQ |
| | | NPTYEQVIYM GTGHAETVKV VYDPERVDLE TILRHFFRII |
| | | DPTSLNRQGN DRGTQYRTGI YYTDASDAAL ITAALAREQS |
| | | KWQKPLVVEN EALDAFYVAE EYHQDYLAKN PNGYCHVDLN |
| | | LVDQPLEKEE FELEMKSGEN TQTMQNLKSE IRINPADYSV |
| | | PSDEELRQKL SPLEYQVTQQ NATERAFTHS YDNLYEPGIY |
| | | VDIVSGEPLF SSDDKYDSGC GWPSFTKPIV PEVVTEHLDT |
| | | TYNMQRIETR SRVADAHLGH VFPDGPRDRG GLRYCINGAS |
| | | LKFIPKAEMA AAGYGDLLPL VSDK |
| 26 | methionine sulfoxide | MHTLFRILST LLFLSLSFFS FSAHSVGVSS QPHVGQRIAK |
| | reductase, | LKDFQDKPAT DYLKKGQPSL VKFWASWCPL CLATLEETRD |
| | Alcaligenaceae | WRLDPDFAGV NIISLASPGY LNEQSPKEFR QWYRGVNFDN |
| | | LPVIVNDGGE LTRAIGISAY PSWGLIDAEG RLQRVIKGHI |
| | | TKEQALALVA DKDYEIKRKT PDFYRPSKDT AQQQKDKANL |
| | | MKTKEIYLAG GCFWGVEAYF ERIPGVVNAV SGYANGKTRQ |
| | | PTYEQVIYMN TGHAETVKVV YDPERIDLET ILRHYLRIID |
| | | PTSLNRQGND RGTQYRTGIY YSEPSDKDII TAVLAREQSK |
| | | WERPIVVENQ PLIAFDEAEE YHQDYLAKNP NGYCHIDLNL |
| | | VDKPLAEEKT PLNLQNGSNT KAMQEHNAQS SITVNPADYH |
| | | VPKEEELRKT LSPLSYQVTQ QNATERAFTH PYDHLFEAGI |
| | | YVDIVSGEPL FSSDDKYDSG CGWPSFTKPI VPEVITEHLD |
| | | TSYNMQRIET RSRVADAHLG HVFPDGPRDR GGLRYCINGA |
| | | ALKFIPKAEM EAAGYGYLLP LVSDK |
| 27 | methionine sulfoxide | MSYKNNQKNS NHEEIKKPRS SSWLKNVSAF SMTTVLSAGI |
| | reductase, | LVACGQMSNA ESSASSSTKS GSQNTGVTSS RDMLPSDMLK |
| | Psychrobacter piscatorii | QMQALPQLTK GLGDTGAAVI DPNKPTLVKF WASWCPLCLG |
| | | TLEETETWRT DPKFSGLNVV TVASPGHLNE KADGEFSTWY |
| | | AGVQADYPKL PVLTDPSGEL INKLGVQVYP SWAILDKNGN |
| | | LVHLVKGNIS AEQAYALAEN AGNGFAELKA GNAKPANAQA |
| | | SDNNKIETIK QKDGVYYNET GKPINTRSIY LAGGCFWGVE |
| | | AYMERVDGVI DAVSGYANGD TANPSYEQVI RGSGHAETVK |
| | | VTYDADKTDL DTILKYYFRV VDPTSLNKQG NDRGVQYRSG |
| | | VYYTDKEDKA VIDAALKRVQ SKYEQKVVVE NEPLDNFYLA |
| | | EMYHQDYLAK NPNGYCHIDL SLADDKPEGA ARTKLAPVET |
| | | IAETLDPKRY AKFDKDALKN TLTKAQYNIT QEAGTERAFS |
| | | HEYDDLFAPG IYVDVVSGEP LFLSTDKYQS GCGWPSFTKP |
| | | IDIQVITQHQ DTAFNMVRTE VRSRVADSHL GHVFPDGPKD |
| | | RGGLRYCING GALQFIPVDV MPQSGYAPLV KLVKS |
| 28 | methionine sulfoxide | MLKQRQLPRF RSLGLSLSVL AVLLGLHSTR VLAAPQSQVA |
| | reductase, Brackiella | PNSDLRSALG QLTTVTGQSG QNYLRADRPT LVKFWASWCP |
| | oedipodis | LCLASLHETS AWSRDQDFAA FNIVSVAAPG YFNELPLEQF |
| | | KHWFAGVDEA DKKGLVVLLN EGGQLTRRLG IAAYPSWALL |
| | | DRQGRLQRIV KGQLSKEQAL GLLTNKDYSL KPAPKSFYKK |
| | | SSASQQDSAT LMNTKTIYLA GGCFWGVEAY FERIPGVVDA |
| | | VSGYANGKSR HPSYEDVVYR NTGHAETVAV TYDPKQINLA |
| | | QLLTHYFRLI NPTSLNQQGN DRGTQYRTGI YYTDTADKAV |
| | | ISRALADLQH HYKAPIVVEN QPLAAFDKAE DYHQDYLAKN |
| | | PNGYCHIDLR QADQPLSQEE LKQVQHIQDA TQTDASAPKS |
| | | PELTPQRFKV PSPEELKKTL SPLAYDVTQN NATERAFTSE |
| | | LDHVFEPGIY VDVVSGEPLF SSTDKFDSGC GWPSFTKPIK |
| | | ADLITEHSDH SYNMIRTEVR SHTANSHLGH VFNDGPKDKG |
| | | GLRYCINGAA LKFIPKDKMQ EAGYGDYLQY VK |
| 29 | methionine sulfoxide | MGKFLKVLFS VFLVAATQIA CSQAKSNSTL SQLKDVDNKS |
| | reductase, Taylorella | FNIDSSKPTL IKFWASWCPL CLGELPDVEN WYKDEAFKGV |
| | asinigenitalis | NLVTIASPSY LSEKKEEAFK NWAKQSGIYK SGSFPIYVDP |
| | | KGSHAKKWGI KVYPSWVLLD KNQQVQRIIK GSISKKQALA |
| | | LINNKDANLM ETEKKYYKES NNGESKIPLR TETIYLAGGC |
| | | FWGVEAYFQK IPGIVDAVSG YANGNVENPH YRLVTTGTTG |
| | | FTETVKITYD IDKIGIQEIL AHYFRIIDPT SLNKQGNDRG |
| | | TQYRTGIYYE KPEYKEIVAK ALEDLQKKYS EPVVVENMQL |
| | | KNFYMAEEYH QDYLIKNPNG YCHIDLSLAD KPLEGVKKMK |
| | | KGFDEASYVK PSDEELRKTL TPEQYRVTQE EGTEFAFSHE |
| | | YDNLFEPGIY VDVVSGEPLF SSDDKYNSGC GWPSFSKPIE |
| | | DDNIHEKKDF KIGYPRTEVR SSAADSHLGH VFNDGPKELG |
| | | GLRYCINGAS LRFIPYSEMK EQGYEEWMDK VKPIKGGATE |
| | | VNKK |
| 30 | methionine sulfoxide | MTKPRLRSHA CAISLGIFAS LSMLSACGKP NDIQTQSVTH |
| | reductase, Moraxella | QDMLPSDTLA QLSALPQLTQ GLGDTGKSVI DPNKPTLVKF |
| | catarrhalis | WASWCPLCLS TLQETHDWRG DPNLAGFNII TVASPTHLNE |
| | | KNTQDFTNWY QVLQADYPNL PVLIDSSGQL IKSLGIQVYP |
| | | SWAILDKNGQ LVYLSKGNLS VEQVSYLAKN PQALNELKAQ |
| | | SHQTAMPTKD KDGVHYNDQG MPLNTKTIYL AGGCFWGVEA |
| | | YFERIDGVVD AVSGYANGDE TLKNPSYEQV IAGSGHAETV |
| | | KVVYDADKMD LDTLLRYYFR IIDPTSVNKQ GNDRGIQYRT |
| | | GVYYTDPSDK AIIDNALNEL QQKYKAPIVV ENLPLSHFAL |
| | | AEDYHQDYLT KNPNGYCHVD LSLANDKIVS KAQTLPKAST |
| | | IQEALDPKRY QAFDKDNLKN TLTKAQYDIT QNAGTERAFS |
| | | HAYDHLFDDG LYVDIVSGEP LFLSTDKYNS GCGWPSFTKP |
| | | IDPQVITEHT DTSYNMVRTE VRSRTADSHL GHVFPDGPKA |
| | | RGGLRYCING DALKFIPKAD MDKHGYGALL PLIKPAQP |
| 31 | methionine sulfoxide | MFLIKNLLEC QLTAIFDALQ IDEKCTMVNF PQSAKNLTIT |
| | reductase, | LLISSLLLLG CQKMNAKENA TYAGAASKAD VLPTDTLATL |
| | Enhydrobacter | QGLSQINPKL GKMGRHVIDP NKPTVVKFWA SWCPLCLATL |
| | aerosaccus | QESDAWAKQY PDMNVISVVS PGHLSEKSSQ DFQTWYTVLA |
| | | KDYANLAVLM DNNGKLIKQF GVQVYPSFAI LDKQGNLLKL |
| | | VKGNLTPTQI QALSDNASND FAELKALNQA KTPYSQTAQA |
| | | HEQANNQASK QAASIKALAP INHNGVYYQA DGKTPIRTHT |
| | | IYLAGGCFWG LEAYMERVDG VVDAISGYAN GNSANPSYEQ |
| | | VIAGSGHAET VKVIYDIDKT NLATLLAYYV RVIDPTSLNK |
| | | QGNDRGAQYR TGIYYTDAND KPIIDKTLAD LAKKYPQKIV |
| | | VENKPLANFY DAENYHQDYL SKNPNGYCHI DINLANQKIP |
| | | VIKSLAPATT VTEALNPSRY QNYDKNVKSR LTQAQYDVTQ |
| | | NAATERAFSH QYDHLFAKGL YVDIVSGEPL FLSTDKYDSG |
| | | CGWPSFTKPI SANVITTSTD SSFNMTRTEV RSRVANSHLG |
| | | HVFDDGPKDK GGLRYCINGD ALQFIALADM QAAGYGALMP |
| | | LVK |
| 32 | methionine sulfoxide | MKKFYKIFLT FLFLIGGTMV FANRRGIENF ELKTLDGKEY |
| | reductase, | TLPKGKKVYL KAWASWCPIC LSSLEELDSF TKEEDRIEIV |
| | Fusobacterium | TVVFPGKSGE MSKEEFKKWY SSLGYKNIKV LVDEKGELLK |
| | mortiferum | KARIRAFPTS IFIDETGEIK GVVPGQLPKE QILKIMGVDS |
| | | QKKEEVVKKE DNVPVTSKNE GQKIEEIYLA GGCFWGVEAY |
| | | MERIYGVVDA VSGYANGKTE NPRYEDVVYR DTGHAETVKV |
| | | TYDSNKISLS TLLEYYFRIV DPTSLNKQGN DRGTQYRTGI |
| | | YYIKAEDEKV VTQALENLQK KYDKKVVIEN KPLENFYLAE |
| | | EYHQDYLKKN PNGYCHIDLN KANDIIVDAS KYKKLSDKEL |
| | | REKLSEKEYR ITQLNDTERA FDNEYWNFFE PGIYVDITTG |
| | | EPLFSSKDKY NSMCGWPSFT KPISEDVVTY HTDRSFNMVR |
| | | TEVRSRVGDA HLGHVFEDGP KDKGGLRYCI NSGALNFIPV |
| | | DEMEKEGYGY LLKLVK |
| 33 | methionine sulfoxide | MKKRFLLIVF AVIFSITACT SKRDVTNSDE KKKDEIRKQI |
| | reductase, Helcococcus | DEIISQHQNE NNDENPNDSI DYSKTKLKTI YLAGGCFWGV |
| | sueciensis | EAYMEKVYGV ADVVSGYANG NTENPTYEDV LYKNTEHAET |
| | | VKVDYDPEKI SLEKILDYYL LVVDPTSLNK QGNDRGTQYR |
| | | SGVYFTDENE RKIIEERLKK EQEKYKDKIV VEVQKLENFY |
| | | EAEEYHQDYL KKNPNGYCHI DISKANEIII DQSKYPKPSD |
| | | EELKKKLTEA QYRVTQENDT EHAFSNEYWD NKEKGIYVDV |
| | | ATGEPLFGST DKYDSGCGWP SFTKPISKEV VTYHKDFSFN |
| | | MERTEVRSRS GDSHLGHVFD DGPKESGGLR FCINSASIRF |
| | | IPLEDMEKEG YGYLTHIIK |
| 34 | methionine sulfoxide | MKKILLLMVL AATLLVTAYI VKANTTHNEL ANSEMTNKEM |
| | reduclase, Eremococcus | THNEMKNDDT RNKIDEIITQ QQKKSADENP NDAVDYSKAE |
| | coleocola | LKTIYLAGGC FWGVEAYLEK VYGVADVVSG YANGDTENPT |
| | | YEDVSYKNSG HAETVKVDYD PARISLEQIL DYYLLVVDPT |
| | | SMNRQGNDRG LQYRSGVYYT DESERKIIEE RLNKEQAKYE |
| | | DKIVVEVEKL DNFYEAEEYH QDYLKKNPNG YCHIDISKAN |
| | | EVIIDQSKYP KPSDEELKKK LTDVQYKVTQ ENDTEHAFSN |
| | | EYWDNKDKGI YVDVATGEPL FSSTDKFDSG CGWPSFSKPI |
| | | AKEVVTYHTD LSYNMKRTEV RSRSGNSHLG HVFEDGPKEL |
| | | GGLRYCINSA SIRFVPLEEM EQEGYGYLTH LIK |
| 3 | TurboLuc | MEAEAERGKL PGKKLPLEVL IELEANARKA GCTRGCLTCL |
| | | SKIKCTAKMK KYIPGRCADY GGDKKIGQAG IVGAIVDIPE |
| | | ISGFKEMEPM EQFIAQVDRC ADCTTGCLKG LANVKCSDLL |
| | | KKWLPGRCAT FADKIQSEVD NIKGLAGD |
| 4 | Peptide 1 | RTTSFAESCKPVQQPSAFGSMKV, |
| | | Wherein C indicates an carbamidomethyl C, and |
| | | Wherein A indicates an oxidized M |
| 5 | Peptide 1, reduced | RTTSFAESCKPVQQPSAFGSMKV |
| | | Wherein C indicates an carbamidomethyl C |
| 6 | Peptide 2 | RTTSFAESCKPVQQPSAFGSMKV |
| | | Wherein S indicates a phosphorylated S, |
| | | Wherein C indicates an carbamidomethyl C, and |
| | | Wherein M indicates an oxidized M |
| 7 | Peptide 2, reduced | RTTSFAESCKPVQQPSAFGSMKV |
| | | Wherein S indicates a phosphorylated S, and |
| | | Wherein C indicates an oxidized C |
| 8 | Peptide 3 | RTESITATSPASMVGGKPGSFRV |
| | | Wherein S indicates a phosphorylated S, and |
| | | Wherein M indicates an oxidized M |
| 9 | Peptide 3, reduced | RTESITATSPASMVGGKPGSFRV |
| | | Wherein S indicates a phosphorylated S |
| 35 | Msr-A, accession # | mteqatfagg cfwctesvfk qidgvtdvvs gyagghvadp |
| | WP_049944603.1 | syeavcreet ghaecvqlty dpeevsyedl lavhftthtp |
| | | ttkdregndv gtqyrsavfy hdeaqretve alieeiepgy |
| | | dsdivtevep letfypaeey hqdyfeknpd qsycqltipp |
| | | kieklkqkha ella |
| 36 | Msr-A, accession # | mateteratl aggcfwciea pmeeldgvhd vtsgyagght |
| | WP_005043086.1 | enptyravcs gdtghaevvq ieydpdriay edlldvlftv |
| | | hdptqlnrqg pdvgtqyrsa ifthdesqhe taaayidald |
| | | aeggyddpvv teiepletfy easeehqnyy eknpedaycs |
| | | fhaqpkiekv rekfaekta |
| 37 | Msr-A, accession # | messqtatfg ggcfwcieaa fkeldgisdv tsgyaggtve |
| | WP_058572480.1 | nptyeqvcsg ttghaeviqv eydpsvvdyd elldvffavh |
| | | dptqlnrqgp dvgtqyrsiv lyhdddqrrl aeayvealdd |
| | | sydddvvtel apfetfyeae ayhqdyfekn pndaycqfha |
| | | spkiekvrek fadklan |
| 38 | Msr-A, accession # | meratfgggc fwcveaafeq legvdsvtsg yagghtedpt |
| | WP_015322392.1 | yeavcsgstg haevvqveyn pdeiayedll evfftvhdpt |
| | | tkdregpdvq sqyrsaiyah deaqletaea fadeleaegl |
| | | yegivteiep ldtfyeaeqy hqnyfeknpn daycsmhaap |
| | | kvetvrekfg envapeh |
| 39 | Msr-A, accession # | msdashddel etatlgggcf wcveavlkel dgvrsvtsgy |
| | WP_015408133.1 | agghvedpsy eavcrgetgh aevvqvafap etiafrdlle |
| | | vfftihtptt lnregpdvgs qyrsavyyhn deqrrvvesv |
| | | igeleplydd divteveple tfypaeeyhq dyfdknpsdt |
| | | yctvnvnpkl sklrekhael la |
| 40 | Msr-A, accession # | meratfgggc fwcteaamke legvdsvtsg yagghtedps |
| | WP_006431385.1 | yrevcsgntg haevvqveyd pdaigydell evffathdpt |
| | | qlnrqgpdvg tqyrsivlyh dddqrtqaea yidaldseyd |
| | | ddvvtelepl etfyraeekh qdyfeknpnd ayctmhaapk |
| | | vekvrekfae nvaaeh |
| 41 | Msr-B, accession # | mseseeelpd kdeewreils deeyrilres gteprfssdl |
| | WP_004963222.1 | idvedegvft cagcgtelfd sdrkfesetg wpsfwdvyqe |
| | | gnvetradns hgmertevic aecgghlghv fddgpepsgk |
| | | rycingaald fese |
| 42 | Msr-B, accession # | msnepattge lpetdeewre vltdeeyeil reqgtepkfs |
| | WP_049996544.1 | gelldqhddg tfvcagcgte lfssdtkfes ktgwpsfsdv |
| | | adegnvelrr dtshgmerte vvcatcgghl ghvfddgpep |
| | | tgkrycinsa algfdgdes |
| 43 | Msr-B, accession # | msdsefslse sewrerlsed ayrvlreqgt eprfsgehvd |
| | WP_007275637.1 | rsddgvyrca gcgtelfdse tkydsncgwp sfyaaedsni |
| | | elrrdlshgm drtevvcstc gghlghvfdd gpeptgkrfc |
| | | insaaldfea dee |
| 44 | Msr-B, accession # | msnepdvptd dgewreeltd eqyrilreag teapfsgeyv |
| | WP_008423757.1 | dhkddgsyac vgcgttlfds etkfdsgcgw psfsdvdddr |
| | | vetrldtshg mrrtevlcan cgghlghvfd dgpeptgkry |
| | | cinsavlefd ge |
| 45 | Msr-B, accession # | mdsklpqtda ewrevltdee yrilreqgte pkfsgehlga |
| | WP_015408129A.1 | dadgvyrcag cgaelfdset kfdsnsgwps fydaeegave |
| | | lredrshgmv rtevvcarce ghlghvfedg pdptgqrycm |
| | | nsvalefdde a |
| 46 | Msr-B, accession # | msdesdhvpt ndeewrerls deeyrilrea gtetpfsgey |
| | WP_007109050.1 | vdhkadgsya cagcgaelfd setkfdsgcg wpsfydaddd |
| | | rietrtdtsh gmrrtevvca ncgghlghvf ddgpeptgkr |
| | | ycinsvalef de |
| 47 | Msr-B, accession # | msetdetptd errsdeslpe tddewrerls deeyeilrer |
| | WP_023395429.1 | gtearfsgeh vdrdddgvye cagcgtvifd sgtkydsgcg |
| | | wpsfyaadds kvtlrdddrh gmsrvevlca ncdghlghvf |
| | | qdgpeptger fcinsvaldf esrerad |

1. A method of preparing a polypeptide sample for separation and/or analysis, comprising contacting the polypeptide sample with at least one methionine sulfoxide reductase (MSR) enzyme under conditions suitable for reducing oxidized methionines in the polypeptide sample.

2. The method of claim 1, wherein at least one methionine sulfoxide reductase enzyme is capable of reducing methionine-S-sulfoxide, or is capable of reducing methionine-R-sulfoxide, or is capable of reducing both methionine-S-sulfoxide and methionine-R-sulfoxide.

3. The method of claim 1, wherein the method comprises contacting the polypeptide sample with at least one methionine sulfoxide reductase enzyme that is capable of reducing methionine-S-sulfoxide and at least one methionine sulfoxide reductase enzyme that is capable of reducing methionine-R-sulfoxide.

4. The method of claim 1, wherein the method comprises contacting the polypeptide sample with at least one methionine sulfoxide reductase enzyme that is capable of reducing both methionine-S-sulfoxide and methionine-R-sulfoxide.

5. (canceled)

6. (canceled)

7. The method of claim 4, wherein the MsrAB enzyme is derived from a methionine sulfoxide reductase from an organism selected from Neisseria, Lautropia, Cardiobacterium, Gammaproteobacteria, Pelistega, Marinospirillum, Basilea, Oligella, Alcagenaceae, Psychrobacter, Brackiella, Taylorella, Moraxella, Enhydrobacter, Fusobacterium, Helcococcus, Paenibacillus, Eremococcus, Methanobrevibacter, Methanomassiliicoccales, Methanocorpusculum, Thermoplasmatales, Methanometylophilus, Methanoculleus, and Methanocella.

8. The method of claim 7, wherein the MsrAB enzyme is derived from a bacterial methionine sulfoxide reductase enzyme.

9. The method of claim 8, wherein the methionine sulfoxide reductase enzyme is derived from a methionine sulfoxide reductase enzyme of Neisseria gonorrhoeae, Neisseria meningitides, Neisseria lactamica, Neisseria polysaccharea, Neisseria flavescens, Neisseria sicca, Neisseria nacacae, or Neisseria mucosa.

10. The method of claim 1, wherein the methionine sulfoxide reductase enzyme comprises an amino acid sequence that is at least 70% identical to a sequence selected from SEQ ID NOs: 10 to 34.

11. (canceled)

12. (canceled)

13. The method of claim 13, further comprising removing the methionine sulfoxide reductase enzyme following reduction of oxidized methionines in the polypeptide sample.

14. The method of claim 1, wherein the methionine sulfoxide reductase enzyme is present at a weight ratio of between 1:100 and 1:2 enzyme:polypeptide.

15. The method of claim 1, wherein the contacting occurs under reducing conditions.

16. The method of claim 15, wherein the contacting occurs in the presence of dithiothreitol (DTT) and/or dithioerythritol (DTE).

17. The method of claim 1, which is a method of preparing a sample for liquid chromatography.

18. The method of claim 17, wherein the method further comprises subjecting polypeptides of the polypeptide sample to liquid chromatography.

19. (canceled)

20. The method of claim 1, which is a method of preparing a sample for capillary electrophoresis.

21. The method of claim 20, wherein the method further comprises subjecting polypeptides of the polypeptide sample to capillary electrophoresis.

22. The method of claim 1, which is a method of preparing a sample for mass spectrometry.

23. (canceled)

24. (canceled)

25. (canceled)

26. The method of claim 1, wherein the method comprises fragmenting the polypeptides by digestion with trypsin, chymotrypsin, AspN, GluC, LysC, LysN, ArgC, proteinase K, or thermolysin, or by chemical cleavage with CNBr.

27.-35. (canceled)

36. A method of producing a mass spectrometry spectra comprising contacting polypeptides of a polypeptide sample with at least one methionine sulfoxide reductase enzyme under conditions suitable for reducing oxidized methionines in the polypeptide sample, and injecting the polypeptides into a liquid chromatograph/mass spectrometer detection system or directly into a mass spectrometer detection system, wherein the polypeptides of the polypeptide sample have not been fragmented.

37.-52. (canceled)

53. A kit comprising at least one methionine sulfoxide reductase enzyme.

54.-66. (canceled)
